(19) United States
(12) Patent Application Publication
(10) Pub. No.: US 2026/0174771 A1
(43) Pub. Date: Jun. 25, 2026

(54) NOVEL METHODS FOR CHEMICAL SYNTHESIS OF GLYCOSYLATED SPHINGOSINES

(71) Applicant: The Regents of the University of California, Oakland, CA (US)

(72) Inventors: Xi CHEN, Davis, CA (US); Hai YU, Woodland, CA (US); Arin GUCCHAIT, Davis, CA (US); Anand Kumar AGRAHARI, Davis, CA (US)

(21) Appl. No.: 19/130,025

(22) PCT Filed: Nov. 20, 2023
(86) PCT No.: PCT/US2023/080479

§ 371 Date: May 14, 2025

Related U.S. Application Data
(60) Provisional application No. 63/427,020, filed on Nov. 21, 2022.

(51) Int. Cl. A61K 31/5377; C07D 401/12

(52) U.S. Cl. CPC A61K 31/5377; C07D 401/12

(57) ABSTRACT

Described herein are novel methods of preparing sphingosines and glycosylated sphingosines. A method of preparing a glycosylated sphingosine as described herein includes an efficient, four-step route for synthesizing lactosylsphingosine (LacboSph) from Garner's aldehyde. Also described herein is a cost-effective method of synthesizing a variety of glycosylated sphingosines, sphingosines and glycosyl acceptors, including both the d18:1 and d20:1 D-erythro-sphingosines and the d18:1 and d20:1 D-erythro-sphingosine glycosyl acceptors, from xylose.

### CROSS-REFERENCE TO RELATED APPLICATION

The present application claims the benefit of and the priority to U.S. Provisional Application No. 63/427,020, filed on Nov. 21, 2022, which is hereby incorporated by reference in its entirety for all purposes.

### STATEMENT REGARDING FEDERALLY FUNDED RESEARCH

This invention was made with government support under Grant Nos. U01GM120419 and R44GM139441, awarded by the National Institutes of Health. The government has certain rights in the invention.

### BACKGROUND

Glycosphingolipids (GSLs) are biologically important glycolipids that contain a special type of lipid named ceramide (Cer). Mammalian ceramides contain a sphingoid base called sphingosine with varied lengths (d18:1 and d20:1 are the most common) which is N-acylated with structurally diverse fatty acyl chains that differ on the length, with or without the presence of additional hydroxyl groups, and the degree of unsaturation. Mammalian GSLs also vary significantly on their glycan components. In fact, five categories of mammalian GSLs are defined based on their neutral core tetrasaccharide structures, namely ganglio-, globo-, isoglobo, lacto-, and neolacto-series. They all share a same lactosylceramide (LacpCer) core.

Ganglio-series GSLs and some structurally simpler GSLs, such as galactosylceramide (GalβCer), its 3-O-sulfated form 3SGalβCer which is also called sulfatide, and its 3-O-sialylated form Neu5Acα2-3GalβCer (GM4) are abundant in mammalian brains.

Glucosylceramide (GlcβCer), gangliosides, and other GSLs have also been found in cells and tissues from different species. Structurally diverse GSLs are valuable standards for research as well as for analysis and quality control of GSL production and are also potential therapeutics.

Lactosylsphingosine (LacβSph) is a key intermediate for glycosyltransferase-based enzymatic extension of the glycan chains of complex glycosphingosines. Previously, it was synthesized by chemical glycosylation of sphingosine acceptors obtained from either phytosphingosine or a partially protected L-serine via multi-step processes. The previously developed synthetic methods were time-consuming and unsuitable for large-scale production.

### SUMMARY

Described herein are novel methods of preparing glycosylated sphingosines. A first method (referred to herein as “Method I”) of preparing a glycosylated sphingosine as described herein comprises the following steps:
(1a) contacting a Garner's aldehyde with an alkyne of the following structure:

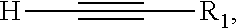

wherein R₁ is a C₁-C₂₅ alkyl,
   in the presence of an organozirconium compound to form a compound of the following structure (Compound A):

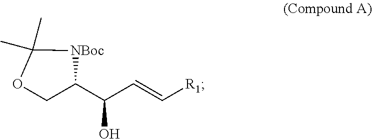

(1b) reacting the hydroxyl group of Compound A with a protecting group reagent and removing the N,O-isopropylidene acetal and tert-butyloxycarbonyl groups to form a compound of the following structure (Compound B):

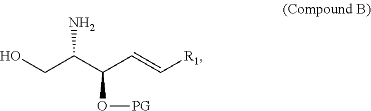

wherein PG is the protecting group;
(1c) converting the amino group of Compound B to an azido group to form a compound of the following structure (Compound C):

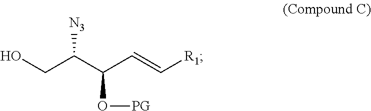

(1d) glycosylating Compound C by a trichloroacetimidate glycosylation reaction to form a compound of the following structure (Compound D):

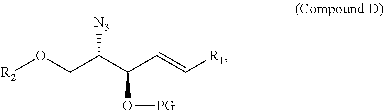

wherein R₂ is a glycosyl group; and
(1e) deprotecting one or more protecting groups present in Compound D and reducing the azido group to form a compound of the following structure (Compound E):

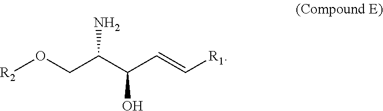

Optionally, the Gamer's aldehyde is (S)-Gamer's aldehyde. The organozirconium compound can be (C₅H₅)₂ZrHCl. In some cases, step (1a) is performed in the presence of a metallic catalyst (e.g., a Zn-based catalyst, such as ZnBr₂). In some cases, the protecting group is benzoyl, and optionally, the protecting group reagent is benzoyl chloride. A purification process is optionally performed after step (1b) and before step (1c). Optionally, a purification process is not performed between steps (1a) and (1b). Step (1c) is optionally performed using triflic azide in the presence of a metal catalyst. The trichloroacetimidate glycosylation reaction optionally comprises reacting Compound C and a glycosylated trichloroacetimidate (e.g., a protected glycosylated trichloroacetimidate) in the presence of a boron catalyst.

In some cases, Compound E is selected from the group consisting of:

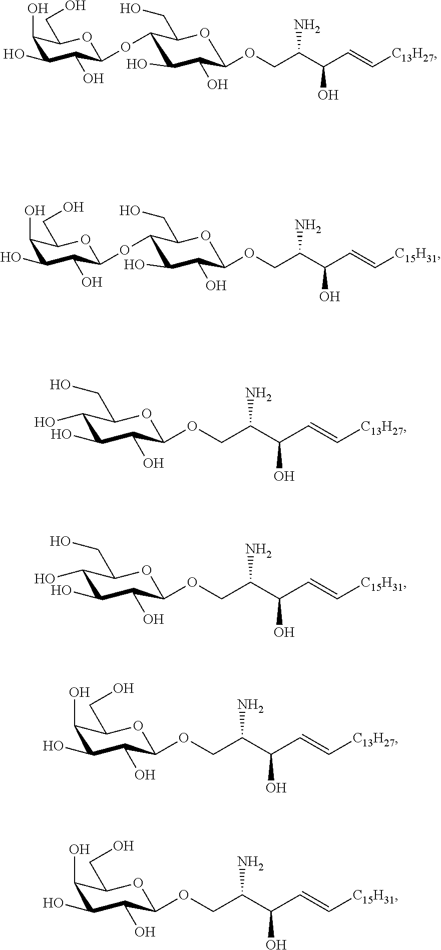

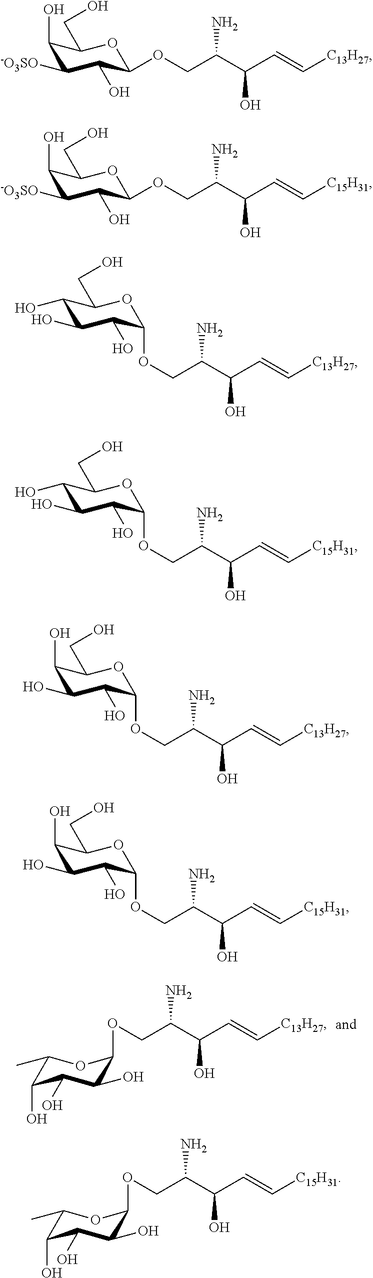

A second method (referred to herein as “Method II”) of preparing a glycosylated sphingosine as described herein comprises the following steps:
(2a) contacting a D-xylose with benzaldehyde dimethyl acetal to form a 3,5-O-benzylidene-D-xylofuranose;
(2b) cleaving the 3,5-O-benzylidene-D-xylofuranose to form a mixture of Compound F and Compound G:

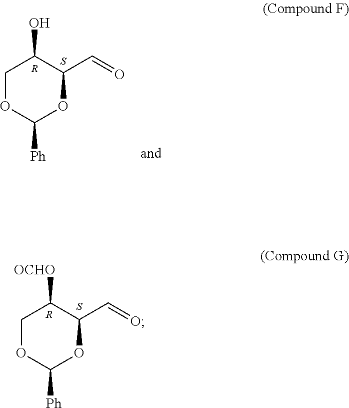

(2c) contacting the mixture of Compound F and Compound G with a triphenylphosphonium reagent of the following structure:

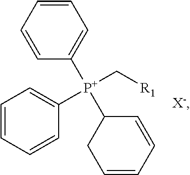

wherein R₁ is a C₁-C₂₅ alkyl and X⁻ is a counterion, to form a compound of the following structure (Compound H):

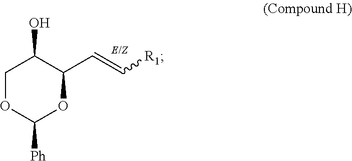

(2d) isolating the E isomer of Compound H to form a compound of the following structure (Compound I):

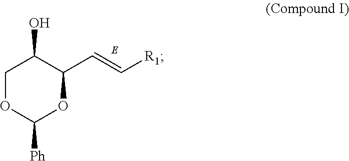

(2e) performing an azidation with S_{N}2 inversion on the hydroxyl group of Compound I to form a compound of the following structure (Compound J):

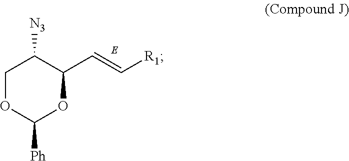

(2f) hydrolyzing Compound J to form a compound of the following structure (Compound K):

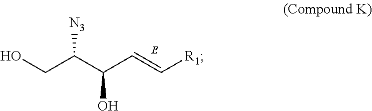

(2g) glycosylating Compound K by a trichloroacetimidate glycosylation reaction to form a compound of the following structure (Compound L):

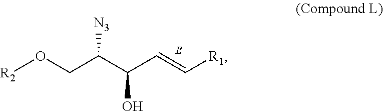

wherein R₂ is a glycosyl group; and
(2h) reducing the azido group in Compound L to form a compound of the following structure (Compound M):

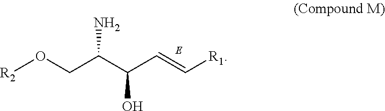

Optionally, the cleaving in step (2b) is performed by a periodate oxidative cleavage reaction. In some examples, the counterion is a halide (e.g., a bromide). In some cases, the contacting in step (2c) is performed in the presence of PhLi with or without LiBr. Optionally, step (2d) further comprises isolating the Z isomer of Compound H from the mixture of E and Z isomers. Optionally, step (2d) further comprises isomerizing the Z isomer of Compound H to form the E isomer (i.e., Compound I). In some cases, prior to step (2e), the E isomer of Compound H is isolated and purified to form Compound I, and the Z isomer of Compound H is isolated, purified, and isomerized to form the E isomer (i.e., Compound I). Optionally, the isomerizing in step (2d) is performed by a photo-isomerization reaction. The light source for the photo-isomerization reaction can be, for example, sunlight or a metal halide lamp.

In some examples, the trichloroacetimidate glycosylation reaction in step (2g) comprises reacting Compound K and a glycosylated trichloroacetimidate in the presence of a promoter. Optionally, the promoter is trimethylsilyl trifluoromethanesulfonate (TMSOTf). The method can further comprise a step of introducing a protecting group after step (2f) and before step (2g) to result in a protected secondary alcohol.

In some cases, Compound M is selected from the group consisting of

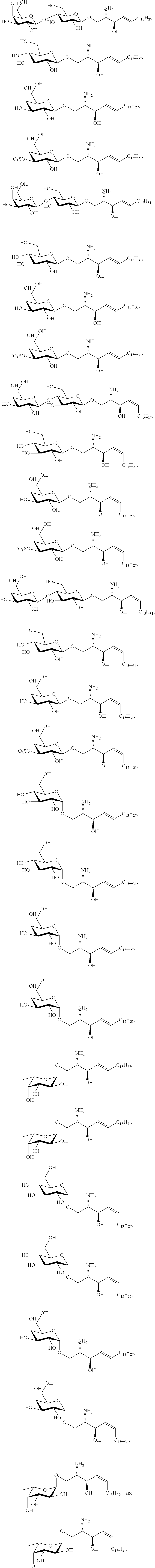

Further described herein are methods of preparing sphingosines. In some examples, a method of preparing a sphingosine comprises:
(3a) contacting a Garner's aldehyde with an alkyne of the following structure:

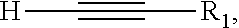

wherein R₁ is a C₁-C₂₅ alkyl,
   in the presence of an organozirconium compound to form a compound of the following structure (Compound A):

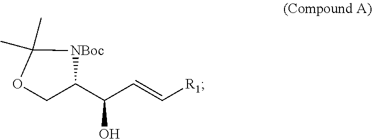

(3b) removing the N,O-isopropylidene acetal and tert-butyloxycarbonyl groups to form a compound of the following structure (Compound N):

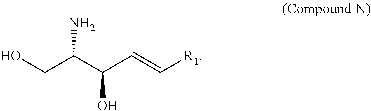

In another method, a method of preparing a sphingosine comprises
(4a) contacting a D-xylose with benzaldehyde dimethyl acetal to form a 3,5-O-benzylidene-D-xylofuranose;
(4b) cleaving the 3,5-O-benzylidene-D-xylofuranose to form a mixture of Compound F and Compound G:

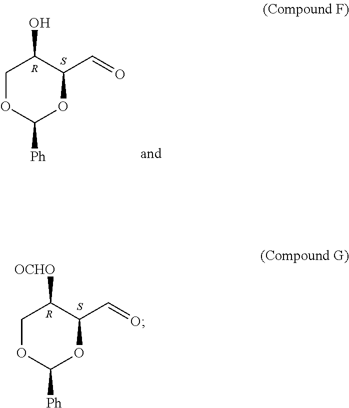

(4c) contacting the mixture of Compound F and Compound G with a triphenylphosphonium reagent of the following structure:

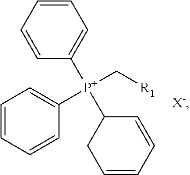

wherein R₁ is a C₁-C₂₅ alkyl and X⁻ is a counterion, to form a compound of the following structure (Compound H):

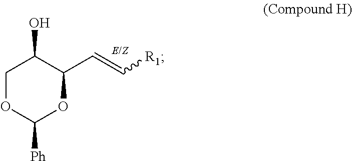

(4d) isolating the E isomer of Compound H to form a compound of the following structure (Compound I):

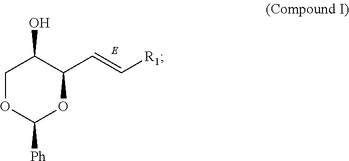

(4e) performing an azidation with S_{N}2 inversion on the hydroxyl group of Compound I to form a compound of the following structure (Compound J):

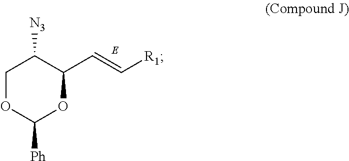

(4f) hydrolyzing Compound J to form a compound of the following structure (Compound K):

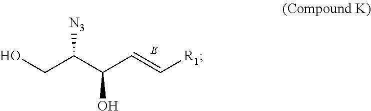

and
(4g) reducing the azido group in Compound L to form a compound of the following structure (Compound N):

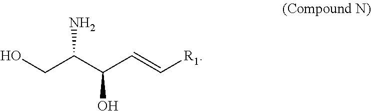

The compound according to Compound N can include, for example, one of the following structures:

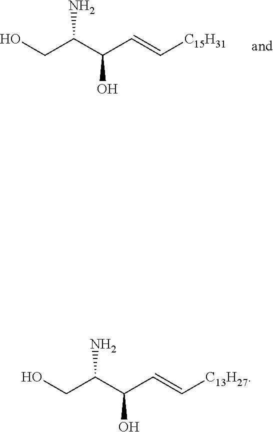

Optionally, step (4d) further comprises isolating the Z isomer of Compound H from the mixture of E and Z isomers. Optionally, step (4d) further comprises isomerizing the Z isomer of Compound H to form the E isomer (i.e., Compound I). In some cases, prior to step (4e), the E isomer of Compound H is isolated and purified to form Compound I, and the Z isomer of Compound H is isolated, purified, and isomerized to form the E isomer (i.e., Compound I). Optionally, the isomerizing in step (4d) is performed by a photo-isomerization reaction. The light source for the photo-isomerization reaction can be, for example, sunlight or a metal halide lamp.

The details of one or more embodiments are set forth in the drawings and the description below. Other features, objects, and advantages will be apparent from the description and drawings, and from the claims.

### DESCRIPTION OF THE DRAWINGS

FIG. 1 contains the ¹H and ¹³C nuclear magnetic resonance (NMR) spectra of Compound I-5.

FIG. 2 contains the ¹H and ¹³C NMR spectra of Compound I-6.

FIG. 3 contains the ¹H and ¹³C NMR spectra of Compound I-8.

FIG. 4 contains the ¹H and ¹³C NMR spectra of Compound I-9.

FIG. 5 contains the ¹H and ¹³C NMR spectra of Compound I-2.

FIG. 6 contains the ¹H and ¹³C NMR spectra of Compound II-5a.

FIG. 7 contains the ¹H and ¹³C NMR spectra of Compound II-5b.

FIG. 8 contains the ¹H and ¹³C NMR spectra of Compound II-5d.

FIG. 9 contains the ¹H and ¹³C NMR spectra of Compound II-7a.

FIG. 10 contains the ¹H and ¹³C NMR spectra of Compound II-7b.

FIG. 11 contains the ¹H and ¹³C NMR spectra of Compound II-8a.

FIG. 12 contains the ¹H and ¹³C NMR spectra of Compound II-8b.

FIG. 13 contains the ¹H and ¹³C NMR spectra of Compound I-10a.

FIG. 14 contains the ¹H and ¹³C NMR spectra of Compound II-10b.

FIG. 15 contains the ¹H and ¹³C NMR spectra of Compound II-11a.

FIG. 16 contains the ¹H and ¹³C NMR spectra of Compound II-11b.

FIG. 17 contains the ¹H and ¹³C NMR spectra of Compound II-12b.

FIG. 18 contains the ¹H and ¹³C NMR spectra of Compound II-14.

FIG. 19 contains the ¹H and ¹³C NMR spectra of Compound II-15.

FIG. 20 contains the ¹H and ¹³C NMR spectra of Compound II-17.

FIG. 21 contains the ¹H and ¹³C NMR spectra of Compound II-18.

FIG. 22 contains the ¹H and ¹³C NMR spectra of Compound II-20.

FIG. 23 contains the ¹H and ¹³C NMR spectra of Compound II-21.

FIG. 24 contains the ¹H and ¹³C NMR spectra of Compound II-22a.

FIG. 25 contains the ¹H and ¹³C NMR spectra of Compound II-22b.

FIG. 26 contains the ¹H and ¹³C NMR spectra of Compound II-24.

FIG. 27 contains the ¹H and ¹³C NMR spectra of Compound II-25.

FIG. 28 contains the ¹H and ¹³C NMR spectra of Compound II-27.

FIG. 29 contains the ¹H and ¹³C NMR spectra of Compound II-28.

FIG. 30 contains the ¹H and ¹³C NMR spectra of Compound II-30.

FIG. 31 contains the ¹H and ¹³C NMR spectra of Compound II-31.

FIG. 32 contains the ¹H and ¹³C NMR spectra of Compound II-12a.

### DETAILED DESCRIPTION

Glycosphingolipids are an important family of glycolipids that play critical roles in various biological processes including protein sorting, signal transduction, membrane raft formation, membrane trafficking, viral and bacterial infection, and cell-cell communications. The structure of both the glycan and the lipid components of glycosphingolipids can vary and the glycosphingolipids isolated from natural sources are a mixture of compounds. Obtaining pure glycosphingolipids is essential to illustrate the biological significance of both the glycan and the lipid (ceramide) portions of the glycosphingolipids at the molecular level and to develop related diagnosis tools (e.g. for detecting different types of cancers, neurological diseases, lysosomal storage diseases, and others) and therapeutics (e.g. cancer vaccines, therapeutics for brain damages, neural damages, and neural degenerative diseases such as Huntington's Disease, Parkinson's Disease, Alzheimer's Disease, and others). Developing efficient synthetic methods to obtain these diverse glycosphingolipids is urgently needed.

Using glucosylsphingosine that can be readily accessible by chemical synthesis as the acceptors for glycosyltransferase-catalyzed reactions is a highly efficient strategy to obtain structurally defined complex glycosphingosines and glycosphingolipids. The chemical synthesis of glycosylsphingosines by chemical glycosylation is achieved by coupling glycosyl donors with appropriate sphingosine acceptors in the presence of a promoter such as a Lewis acid. The reported routes of synthesizing sphingosine acceptors for chemical glycosylation used expensive starting materials and reagents and the processes are long.

Described herein are two chemical synthetic methods for producing partially protected sphingosines which are used as acceptors for chemical glycosylation for the synthesis of relatively simple glycosylsphingosines. These synthetic routes are shorter and are more cost effective. They are suitable for large-scale production of the desired partially protected and the desired relatively simple glycosylsphingosines that are ready for synthesizing more complex glycosylsphingosines by enzymatic glycosylation.

One approach (Method I) uses a commercially available L-serine-derived aldehyde which is called Ganer's aldehyde. Only four steps are needed to produce the desired sphingosine acceptor for chemical glycosylation. This approach synthesizes, for example, the C18-sphingosine glycosyl acceptor by reacting the Garner's aldehyde with 1-pentadecyne. It can also be used to synthesize, for example, the C20-sphingosine glycosyl acceptor by reacting the Garner's aldehyde with 1-heptadecyne. By coupling the sphingosine glycosyl acceptor with suitable glycosyl donors, different glycosylsphingosines, such as lactosyl sphingosines, glucosylsphingosines with an alpha or a beta-glycosidic linkage, galactosylsphingosines with an alpha or a beta-glycosidic linkage, 3-O-sulfogalactosylsphingosines, and fucosylsphingosines can be readily produced by chemical glycosylation followed by deprotection.

Another approach (Method II) uses inexpensive D-xylose as the starting material, with only five to six steps to produce the desired sphingosine acceptor for chemical glycosylation. Again, this approach can access a variety of sphingosines, including both C18-sphingosine and C20-sphingosine, with the naturally occurring trans-alkene form. This method can also access the synthetic cis-alkene form of the sphingosines. The sphingosines prepared according to the methods described herein can be used to synthesize different glycosylsphingosines such as lactosyl sphingosines, glucosylsphingosines with an alpha or a beta-glycosidic linkage, galactosylsphingosines with an alpha or a beta-glycosidic linkage, 3-O-sulfogalactosylsphingosines, and fucosylsphingosines.

Method I: Synthesis of Lactosylsphingosine (LacβSph) from Garner's Aldehyde A method of preparing a glycosylated sphingosine as described herein includes an efficient, four-step route for synthesizing lactosylsphingosine (LacβSph) from Gamer's aldehyde. A first step in the method includes contacting a Garner's aldehyde (e.g., (S)-Garner's aldehyde) with an alkyne of the following structure:

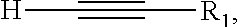

wherein R₁ is a C₁-C₂₅ alkyl. Optionally, R₁ is C₁-C₂₃ alkyl. C₃-C₂₁ alkyl. C₅-C₂₁ alkyl, or C₇-C₁₉ alkyl. The contacting step can be performed in the presence of an organozirconium compound to form a compound of the following structure (Compound A):

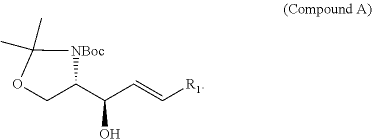

Optionally, the organometallic compound is (C₅H₅)₂ZrHCl, though any suitable organometallic compound can be used in the reaction. In some cases, the contacting step is performed in the presence of a metallic catalyst (e.g., a Zn-based catalyst, such as ZnBr₂). In some cases, Compound A is not purified after its formation and is used without purification in the next step.

The hydroxyl group of Compound A is then reacted with a protecting group reagent to protect the secondary alcohol. In some cases, the protecting group is benzoyl, and optionally, the protecting group reagent is benzoyl chloride. The NO-isopropylidene acetal and tert-butyloxycarbonyl groups are removed to form a compound of the following structure (Compound B):

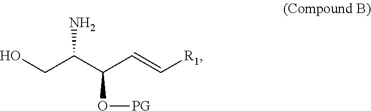

wherein PG is the protecting group.

A purification process is optionally performed to purify Compound B. The amino group of Compound B is then converted to an azido group to form a compound of the following structure (Compound C):

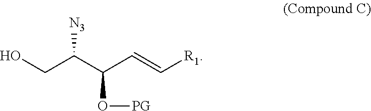

The step of converting the amino group of Compound B to an azido group is optionally performed using triflic azide in the presence of a metal catalyst.

Compound C is then glycosylated using a trichloroacetimidate glycosylation reaction to form a compound of the following structure (Compound D):

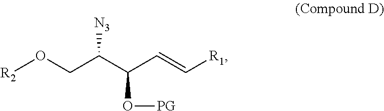

wherein R₂ is a glycosyl group.

The trichloroacetimidate glycosylation reaction optionally comprises reacting Compound C and a glycosylated trichloroacetimidate (e.g., a protected glycosylated trichloroacetimidate) in the presence of a boron catalyst.

Following the trichloroacetimidate glycosylation reaction, one or more protecting groups present in Compound D are removed (i.e., deprotected) and the azido group is reduced to form a compound of the following structure (Compound E):

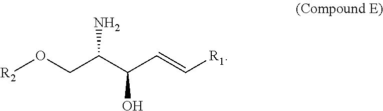

The method described herein can be used to prepare a variety of compounds according to Compound E. In some cases, Compound E is selected from the group consisting of:

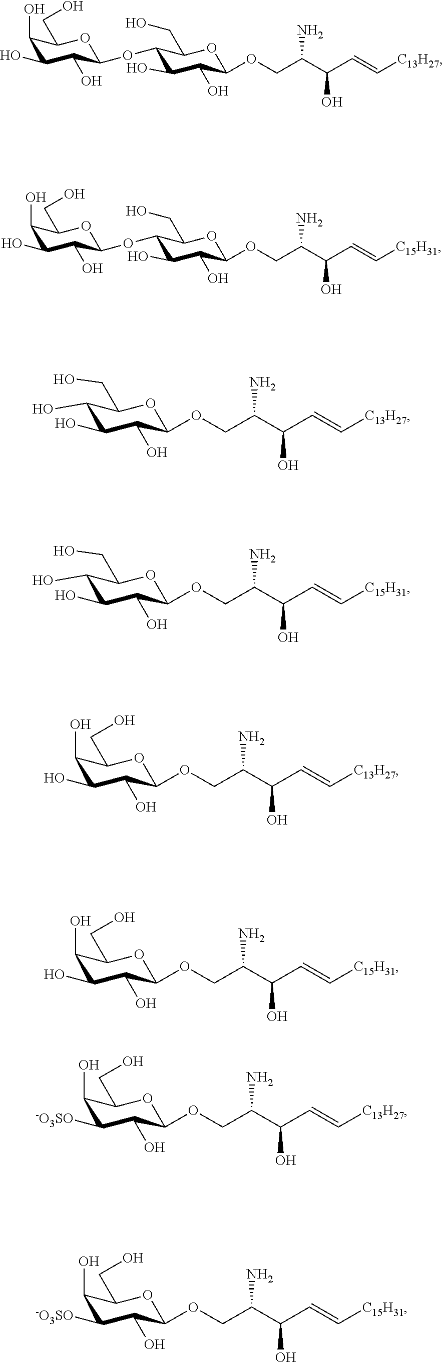

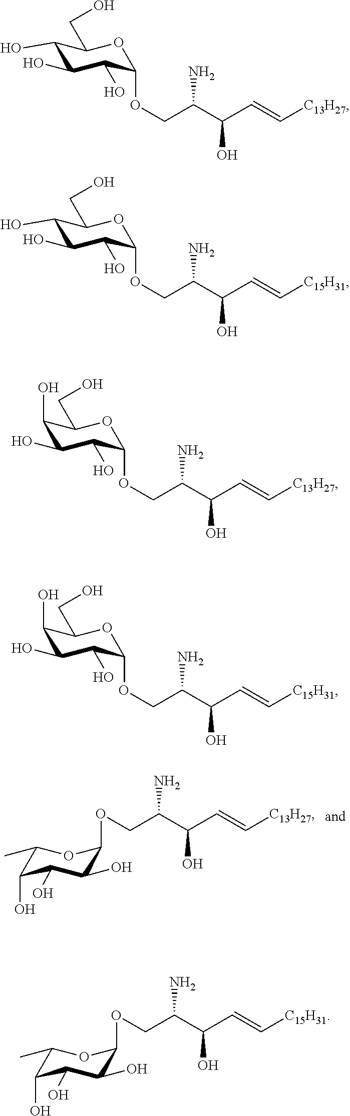

Method II: Synthesis of Glycosyl Acceptors from Xylose

Also described herein is a cost-effective method of synthesizing a variety of glycosyl acceptors, including both the d18:1 and d20:1 D-erythro-sphingosine glycosyl acceptors, from xylose (e.g., D-xylose).

A first step in the method includes contacting a D-xylose with benzaldehyde dimethyl acetal to form a 3,5-O-benzylidene-D-xylofuranose. The 3,5-O-benzylidene-D-xylofuranose is then cleaved to form a mixture of Compound F and Compound G:

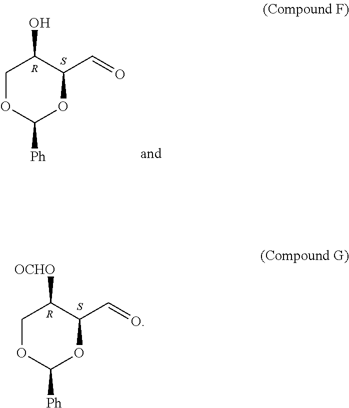

Optionally, the cleaving is performed by a periodate oxidative cleavage reaction.

The mixture of Compound F and Compound G is then contacted with a tnphenylphosphonium reagent of the following structure:

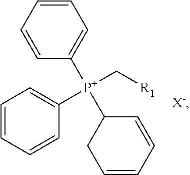

wherein R₁ is a C₁-C₂₅ alkyl and X⁻ is a counterion. In some examples, the counterion is a halide (e.g., a bromide). In some cases, the contacting of the Compound F/Compound G mixture with the triphenylphosphonium reagent is performed in the presence of PhLi with LiBr. In other cases, the contacting of the Compound F/Compound G mixture with the triphenylphosphonium reagent is performed in the presence of PhLi without LiBr. The reaction of the Compound F/Compound G mixture with the triphenylphosphonium reagent results in a compound of the following structure (Compound H):

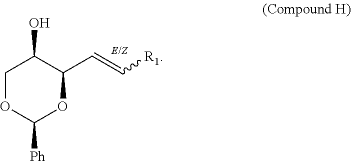

Compound H includes a mixture of E and Z isomers.

The E isomer is then isolated from the mixture of E and Z isomers of Compound H to form a compound of the following structure (Compound I):

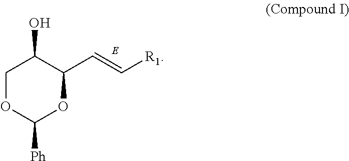

Optionally, the Z isomer is isolated from the mixture of E and Z isomers of Compound H. Optionally, the Z isomer of Compound H is isomerized to form the E isomer (i.e., Compound I). Optionally, the isomerizing is performed by a photo-isomerization reaction. The light source for the photo-isomerization reaction can be, for example, sunlight or a metal halide lamp. In some cases, the E isomer of Compound H is isolated and purified to form Compound I, and the Z isomer of Compound H is isolated, purified, and isomerized to form the E isomer (i.e., Compound I).

An azidation with S_{N}2 inversion on the hydroxyl group of Compound I is then performed, forming a compound of the following structure (Compound J):

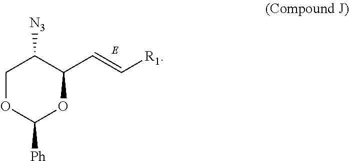

Optionally, the azidation includes a step of converting the hydroxyl group into a leaving group, such as by modifying the hydroxyl group with a mesylate group, a tosylate group, a triflate, a sulfonate, or the like, followed by introducing an azide-containing compound to replace the modified hydroxyl via a nucleophilic substitution (with inversion at the relevant stereocenter).

Compound J is then hydrolyzed to form a compound of the following structure (Compound K):

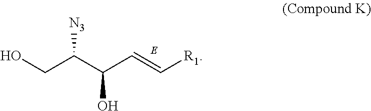

Optionally, the method can further comprise a step of introducing one or more protecting groups to Compound K. Optionally, the secondary alcohol is selectively protected to form a protected secondary alcohol.

Compound K (with or without the protecting group on the secondary alcohol) is then glycosylated using, for example, a trichloroacetimidate glycosylation reaction to form a compound of the following structure (Compound L):

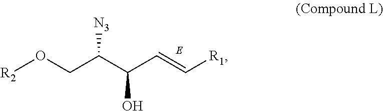

wherein R₂ is a glycosyl group. In some examples, the trichloroacetimidate glycosylation reaction comprises reacting Compound K and a glycosylated trichloroacetimidate in the presence of a promoter. Optionally, the promoter is TMSOTf.

The azido group in Compound L is then reduced to form a compound of the following structure (Compound M):

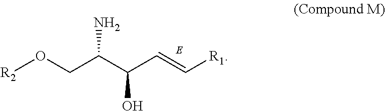

The method described herein can be used to prepare a variety of compounds according to Compound M. In some cases, Compound M is selected from the group consisting of:

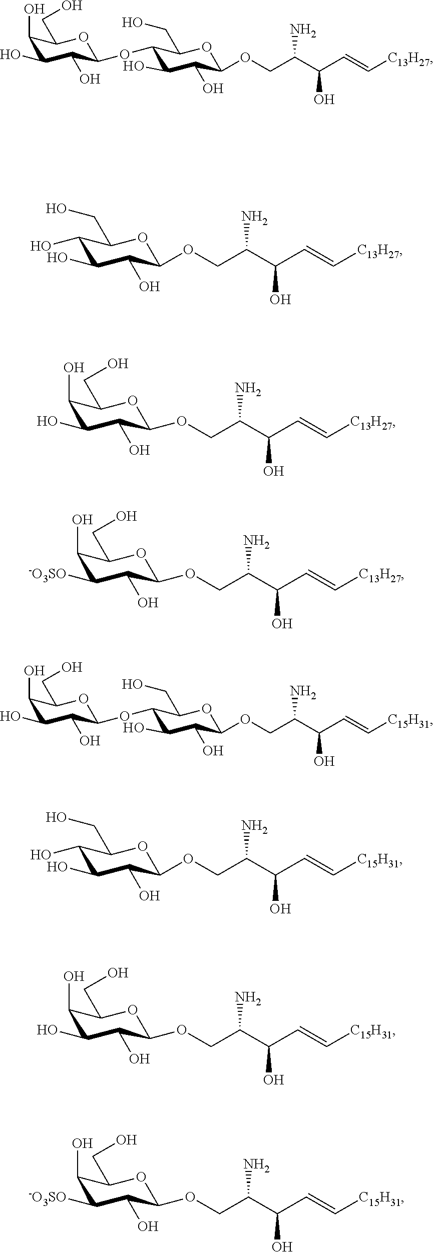

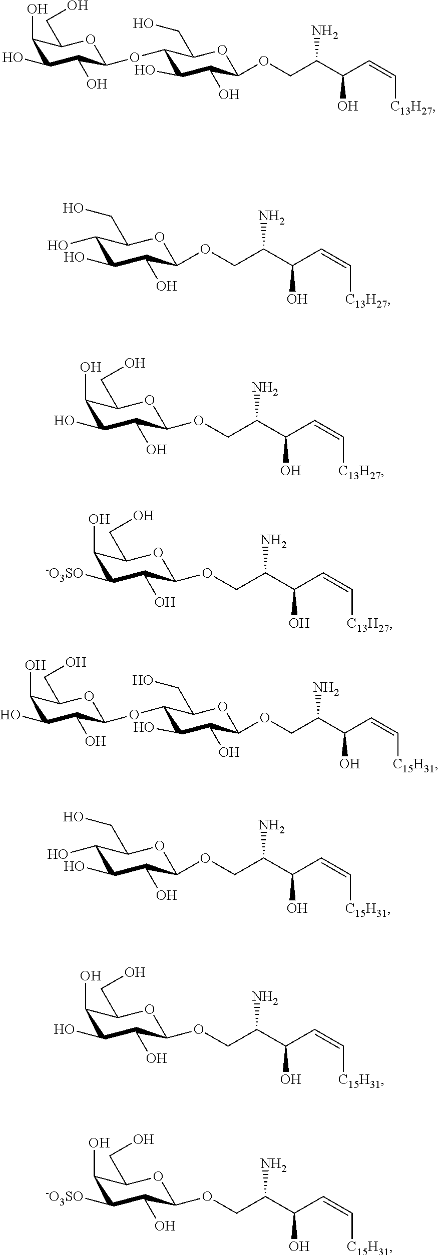

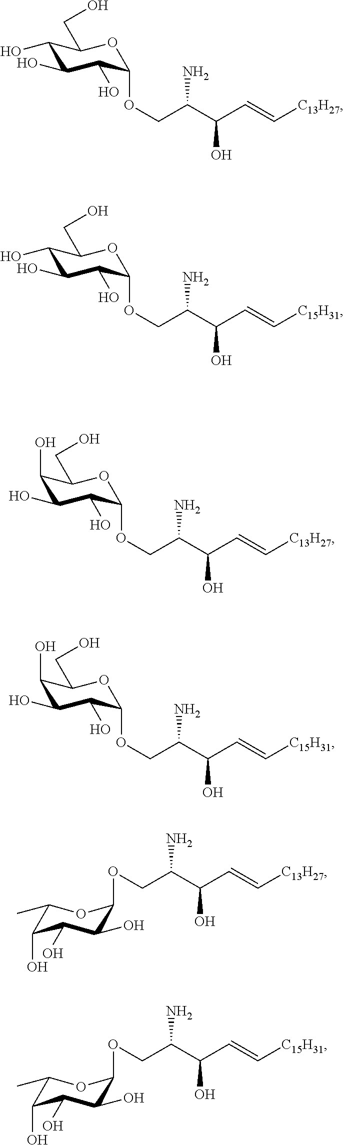

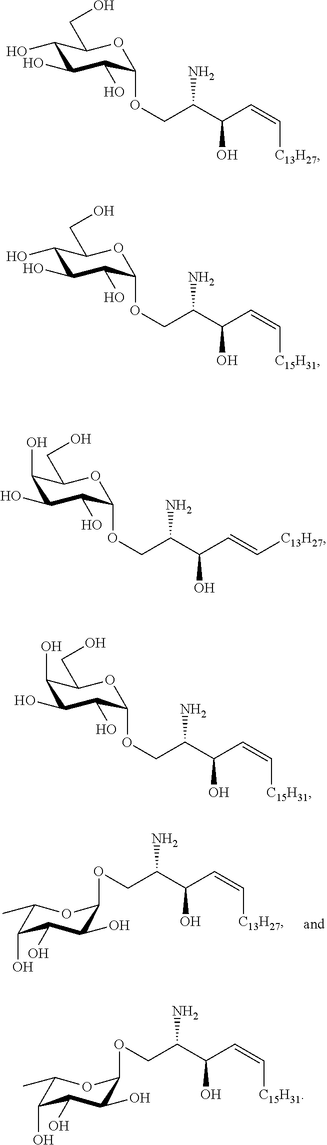

Further described herein are methods of preparing sphingosines. In some examples, a method of preparing a sphingosine includes a step of contacting a Garner's aldehyde with an alkyne of the following structure:

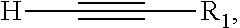

in the presence of an organozirconium compound to form a compound of the following structure (Compound A):

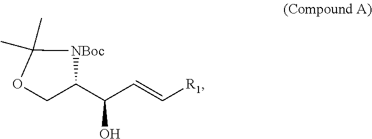

as described above.

The N,O-isopropylidene acetal and tert-butyloxycarbonyl groups are removed to form a compound of the following structure (Compound N):

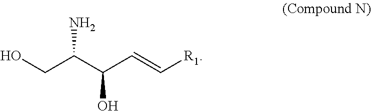

In another method, a method of preparing a sphingosine includes contacting a D-xylose with benzaldehyde dimethyl acetal to form a 3,5-O-benzylidene-D-xylofuranose. The 3,5-O-benzylidene-D-xylofuranose is then cleaved to form a mixture of Compound F and Compound G:

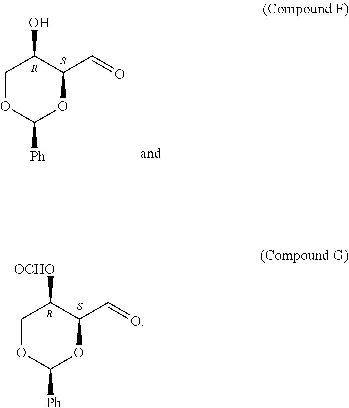

Optionally, the cleaving is performed by a periodate oxidative cleavage reaction.

The mixture of Compound F and Compound G is then contacted with a triphenylphosphonium reagent of the following structure:

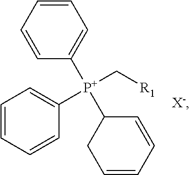

wherein R₁ is a C₁-C₂₅ alkyl and X⁻ is a counterion. In some examples, the counterion is a halide (e.g., a bromide). In some cases, the contacting of the Compound F/Compound G mixture with the triphenylphosphonium reagent is performed in the presence of PhLi with LiBr. In other cases, the contacting of the Compound F/Compound G mixture with the triphenylphosphonium reagent is performed in the presence of PhLi without LiBr. The reaction of the Compound F/Compound G mixture with the triphenylphosphonium reagent results in a compound of the following structure (Compound H) to form a compound of the following structure (Compound H):

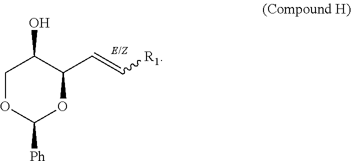

Compound H includes a mixture of E and Z isomers.

The E isomer of Compound H is then isolated to form a compound of the following structure (Compound I):

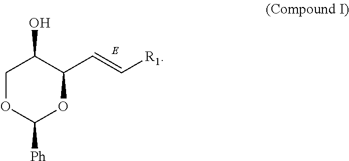

Optionally, the Z isomer is isolated from the mixture of E and Z isomers of Compound H. Optionally, the Z isomer of Compound H is isomerized to form the E isomer (i.e., Compound I). Optionally, the isomerizing is performed by a photo-isomerization reaction. The light source for the photo-isomerization reaction can be, for example, sunlight or a metal halide lamp. In some cases, the E isomer of Compound H is isolated and purified to form Compound I, and the Z isomer of Compound H is isolated, purified, and isomerized to form the E isomer (i.e., Compound I).

An azidation, with S_{N}2 inversion on the hydroxyl group of Compound I is performed, forming a compound of the following structure (Compound J):

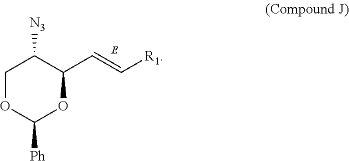

Optionally, the azidation includes a step of converting the hydroxyl group into a leaving group, such as by modifying the hydroxyl group with a mesylate group, a tosylate group, a triflate, a sulfonate, or the like, followed by introducing an azide-containing compound to replace the modified hydroxyl via a nucleophilic substitution (with inversion at the relevant stereocenter).

Compound J is then hydrolyzed to form a compound of the following structure (Compound K):

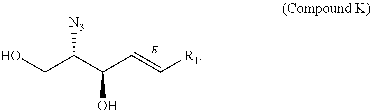

The azido group in Compound K is then reduced to form a compound of the following structure (Compound N):

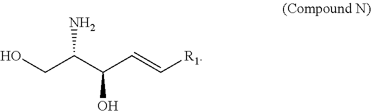

The compound according to Compound N can include, for example, one of the following structures:

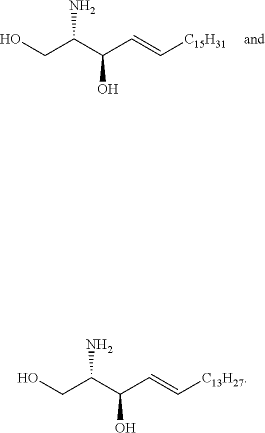

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₅ alkyl, C₃-C₂₅ alkenyl, and C₃-C₂₅ alkynyl. Additional ranges of these groups useful with the compounds and methods described herein include C₁-C₁₅ alkyl, C₃-C₁₅ alkenyl, C₃-C₁₅ alkynyl, C₁-C₇ alkyl, C₃-C₇ alkenyl. C₃-C₇ alkynyl, C₁-C₅ alkyl, C₃-C₅ alkenyl, and C₃-C₅ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₅ heteroalkyl, C₃-C₂₅ heteroalkenyl, and C₃-C₂₅ heteroalkynyl. Additional ranges of these groups useful with the compounds and methods described herein include C₁-C₁₅ heteroalkyl, C₃-C₁₅ heteroalkenyl, C₃-C₁₅ heteroalkynyl, C₁-C₇ heteroalkyl, C₃-C₇ heteroalkenyl, C₃-C₇ heteroalkynyl, C₁-C₅ heteroalkyl, C₃-C₅ heteroalkenyl, and C₃-C₅ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₅ cycloalkyl, C₃-C₂₅ cycloalkenyl, and C₃-C₂₅ cycloalkynyl. Additional ranges of these groups useful with the compounds and methods described herein include C₅-C₁₅ cycloalkyl, C₅-C₁₅ cycloalkenyl, C₅-C₁₅ cycloalkynyl, C₅-C₇ cycloalkyl, C₅-C₇ cycloalkenyl, and C₅-C₇ cycloalkynyl.

The terms heterocycloalkyl, heterocycloalkenyl, and heterocycloalkynyl are defined similarly as cycloalkyl, cycloalkenyl, and cycloalkynyl, but can contain 0, S. or N heteroatoms or combinations thereof within the cyclic backbone. Ranges of these groups useful with the compounds and methods described herein include C₃-C₂₅ heterocycloalkyl, C₃-C₂₅ heterocycloalkenyl, and C₃-C₂₅ heterocycloalkynyl. Additional ranges of these groups useful with the compounds and methods described herein include C₅-C₁₅ heterocycloalkyl. C₅-C₁₅ heterocycloalkenyl, C₅-C₁₅ heterocycloalkynyl, C₅-C₇ heterocycloalkyl, C₅-C₇ heterocycloalkenyl, and C₅-C₇ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bound through a single, terminal ether linkage. The term aryloxy as used herein is an aryl group bound through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bound through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, cycloalkoxy, aryloxy, amino, alkyl, alkenyl, alknyl, aryl, heteroalkyl, heteroalkenyl, heteroalkynyl, heteroaryl, cycloalkyl, or heterocycloalkyl group to a position attached to the main chain of the alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens. i.e., commensurate with its saturation level, with no substitutions, e.g., linear decane (—(CH₂)₉—CH₃).

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on the compounds described herein include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

Exemplary procedures for synthesizing the compounds as described herein are provided in Examples 1 and 2 below.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Four-Step Chemical Synthesis of Lactosylsphingosine

An efficient, four-step route for the synthesis of lactosylsphingosine (LacβSph) is provided herein, as shown below in Scheme 1.

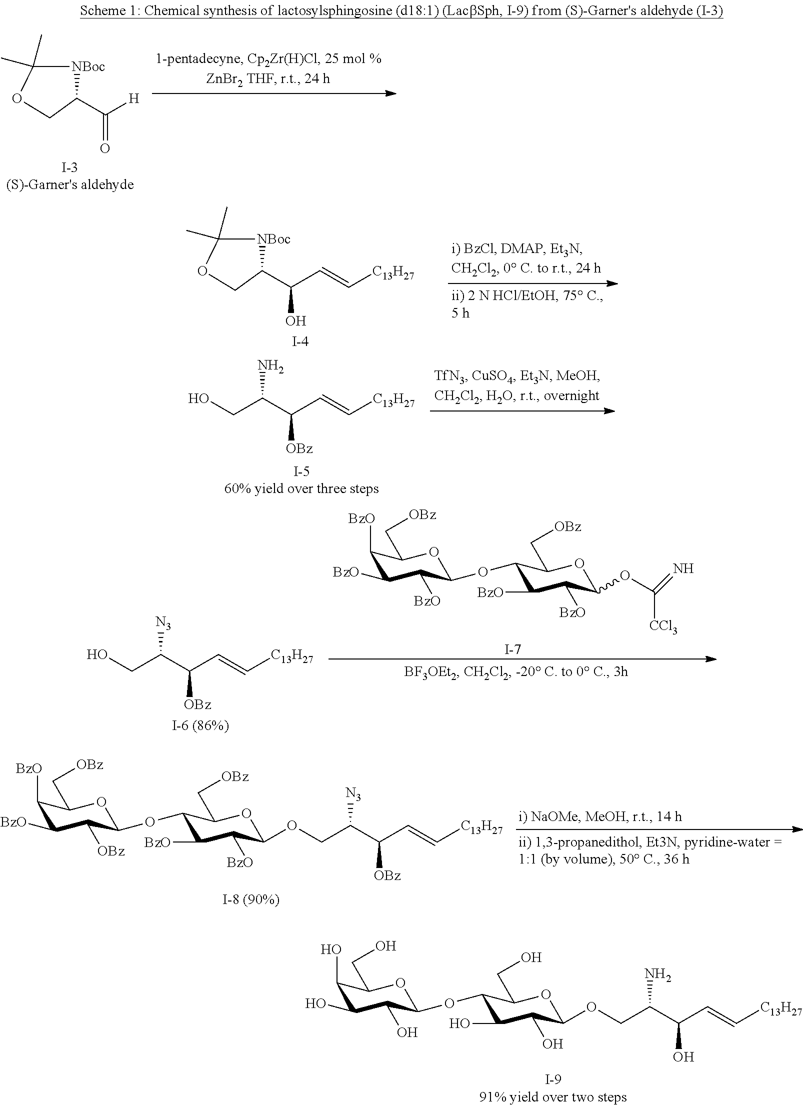

Briefly, (S)-1,1-dimethylethyl 4-formyl-2,2-dimethyloxazolidine-3-carboxylate [(S)-Garner's aldehyde, I-3] was identified as a starting material. Beneficially, it is commercially available (e.g., from Fisher Scientific; Waltham, MA) and is much more affordable than D-erythro-sphingosine (d18:1). It can also be readily prepared from L-serine.

From the (S)-Gamer's aldehyde (I-3), the desired sphingosine glycosyl acceptor (I-6) was synthesized in 1.37 gram in an overall yield of 52% with a short four-step route (Scheme 1). Briefly, (S)-Gamer's aldehyde (I-3) was reacted with 1-(E)-pentadecenylzirconocene chloride formed from 1-pentadecyne and zirconocene chloride hydride Cp₂Zr(H)Cl (Schwartz' reagent) with a catalytic amount (25 mol %) of ZnBr₂ in tetrahydrofuran (THF). Diastereoselective formation of the anti-adduct (I-4) with the desired R-configuration at the newly formed chiral carbon center (C-3) and the E-alkene isomer was achieved. A ratio of 12:1 favoring the anti-versus the syn-product was determined by ¹H-NMR. The product isomers were not separated in this step. Benzoylation of the hydroxyl group in the partially protected sphingosine intermediate I-4 by benzoyl chloride (BzCl) in dichloromethane (CH₂Cl₂), and removal of both N,O-isopropylidene acetal and tert-butyloxycarbonyl (Boc) protection groups by incubating with 2 N hydrochloric acid in ethanol at 75° C. led to the formation of compound I-5 with a 60% isolated yield over three steps. Its amino group was converted to an azido group by reacting with freshly prepared triflic azide in the presence of catalytic CuSO₄ and triethylamine to form the desired 2-azido-3-O-benzoyl sphingosine (I-6) in 86% yield in multi-gram scales.

Glycosylation of the glycosyl acceptor I-6 with per-O-benzoyl lactosyl trichloroacetimidate glycosyl donor (I-7) in the presence of BF₃·OEt₂ in CH₂Cl₂ at −20° C. produced the protected lactoside I-8 in 90% yield. Removal of all benzoyl protecting groups using NaOMe/MeOH and selective reduction of the azido group by 1,3-propanedithiol and triethylamine produced the desired LacPSph (d18:1) I-9 (1.05 g) in an excellent 91% yield.

### General Methods

All chemicals were obtained from commercial suppliers and used without further purification. ¹H NMR (600 Hz) and ¹³C NMR (150 MHz) spectra were recorded on a Bruker Avance-600 Spectrometer (Bruker: Billerica. MA). High-resolution electrospray ionization (ESI) mass spectra were recorded using a Thermo Scientific Q Exactive HF Orbitrap Mass Spectrometer (Thermo Fisher Scientific; Waltham, MA). Thin-layer chromatography (TLC, Sorbent Technologies; Norcross, GA) was performed on silica gel plates using anisaldehyde sugar stain for detection.

### (2S,3R,E)-2-Amino-3-O-benzoyloxy-octadec-4-ene-1-ol (I-5)

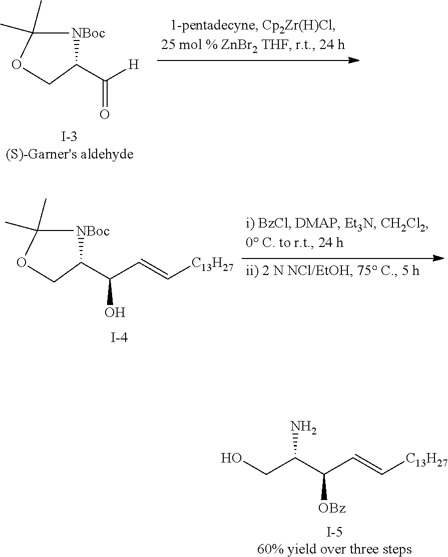

1-Pentadecyne (20 mL, 76.1 mmol) in anhydrous THF (105 mL) was added into a suspension of Cp₂Zr(H)Cl (19.63 g, 76.1 mmol) in anhydrous THF (75 mL) with stirring under argon at 0° C. by incubation in an ice-water bath. The mixture was stirred at room temperature for 1 hour and then cooled to 0° C. by incubating in an ice-water bath. To the resulting yellow solution, Gamer's aldehyde I-3 (8.72 g, 38.0 mmol) in THF (105 mL) was added followed by the addition of ZnBr₂ (4.28 g, 19.0 mmol, dried under vacuum overnight before use). The mixture was stirred at room temperature for 24 hours, then diluted with EtOAc (200 mL) and aq. potassium sodium tartrate (200 mL), and stirred for 15 minutes. The resulting suspension was filtered through a pad of Celite and washed thoroughly with EtOAc.

The combined filtrate was washed with H₂O and then brine (saturated solution of NaCl in water). The combined organic layers were dried over Na₂SO₄ and filtered. The filtrate was concentrated and passed through a silica gel column chromatography using EtOAc:hexane=1:7 to 1:5 (by volume) as an eluant to produce the crude product I-4 (15.38 grams) containing a small amount of syn isomer.

To a solution of the crude product obtained above (13.6 g, 30.9 mmol) in anhydrous CH₂Cl₂ (150 mL) at 0° C., triethylamine (30 mL, 216.5 mmol), 4-dimethyl amino pyridine (DMAP) (378 mg, 3.1 mmol) and BzCl (7.19 mL, 61.9 mmol) were added. The reaction mixture was stirred at room temperature for 24 hours. After the starting material was completely consumed as monitored by TLC analysis, the reaction mixture was washed with 1 N HCl, sat. NaHCO₃, and then brine. The combined organic layers were collected and dried over Na₂SO₄ and filtered. The filtrate was concentrated under reduced pressure and the residue was purified by passing through a silica gel column using toluene:EtOAc=40:1 (by volume) as an eluant. The fractions were collected and concentrated to obtain the benzoylated intermediate (16.8 g) as a colorless oil.

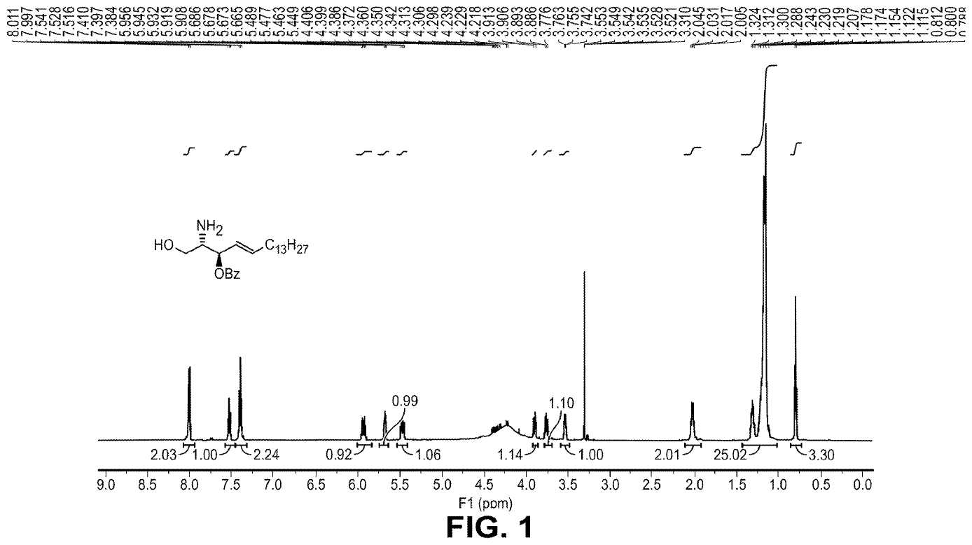

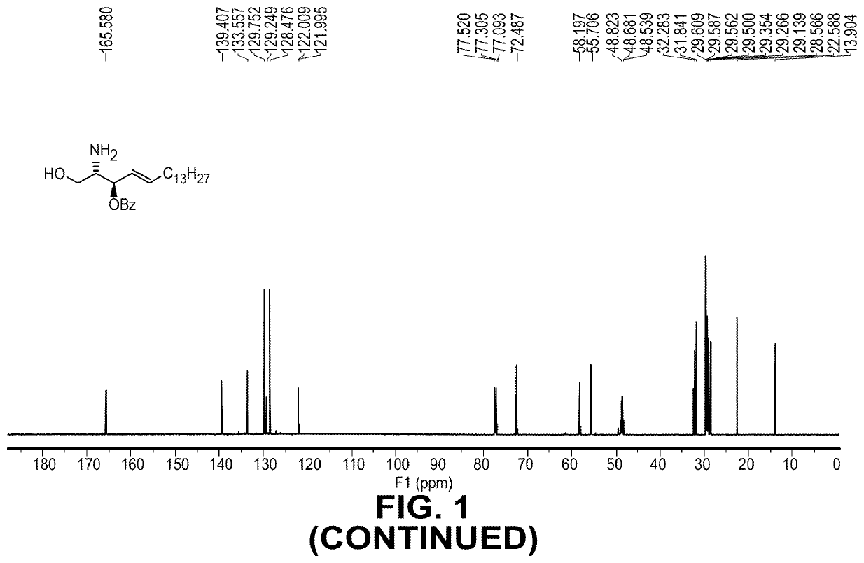

The intermediate obtained above (5.0 g) was dissolved in EtOH (16 mL), and aqueous 2 N hydrochloric acid (4 mL) was added. The mixture was stirred at 75° C. for 5 hours. CHCl₃/MeOH (100 mL, 7:1, by volume) and water (100 mL) were added into the reaction mixture, the layers were separated, and the aqueous phase was extracted the CHCl₃/MeOH (7:1, 4×80 mL). The combined organic phase was evaporated and the residue was purified by silica gel column chromatography using CH₂Cl₂:CH₃OH=30:1 to 20:1 then 10:1 (by volume) as an eluant to obtain compound I-5 (2.42 g, 60% yield over three steps) as a white solid. ¹H NMR (600 MHz, CD₃OD) δ 8.00 (d, J=8.4 Hz, 2H), 7.53 (m, J=7.8 Hz, 1H), 7.40 (t, J=7.8 Hz, 2H), 5.93 (dt, J=14.4, 6.6 Hz, 1H), 5.69-5.67 (m, 1H), 5.47 (dd, J=15.6, 7.2 Hz, 1H), 3.90 (dd, J=12.0, 4.2 Hz, 1H), 3.76 (dd, J=12.0, 7.8 Hz, 1H), 3.55-3.52 (m, 1H), 2.05-2.00 (m, 2H), 1.34-1.12 (m, 25H), 0.80 (t, J=7.2 Hz, 3H). ¹³C NMR (150 MHz, CD₃OD) δ 165.58, 139.41, 133.56, 129.75, 129.25, 128.48, 122.01, 121.99, 72.49, 58.20, 55.71, 32.28, 31.84, 29.61, 29.59, 29.56, 29.50, 29.35, 29.27, 29.14, 28.57, 22.59, 13.90. High resolution mass spectrometry (HRMS) (ESI-Orbitrap) m/z: [M+Na]⁺ calculated for C₂₅H₄₁NO₃Na 426.2984, found 426.2967. See FIG. 1 for ¹H and ¹³C NMR spectra of compound I-5.

### (2S,3R,E)-2-Azido-3-O-benzoyloxy-octadec-4-ene-1-ol (I-6)

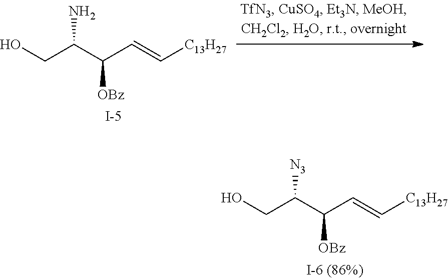

Preparation of TfN₃: To a solution of NaN₃ (1.45 g, 22.3 mmol) in water (4 mL), CH₂Cl₂ (4 mL) was added. The reaction mixture was cooled down to 0° C. in an ice-water bath. To the vigorously stirred mixture, Tf₂O (1.85 mL, 11.2 mmol) was added dropwise. After the mixture was stirred at 0° C. for 2 hours, sat. NaHCO₃ (2 mL) was added. The mixture was stirred at room temperature for 5 minutes. The aqueous phase was separated and extracted with CH₂Cl₂ (3 mL) twice. The combined solution of the organic layers containing TfN₃ was used immediately in the next step.

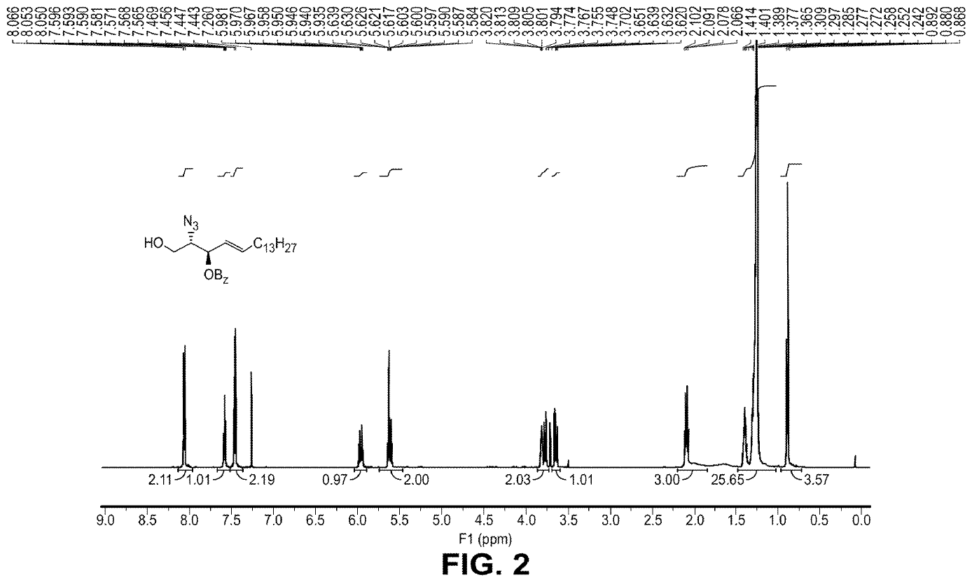

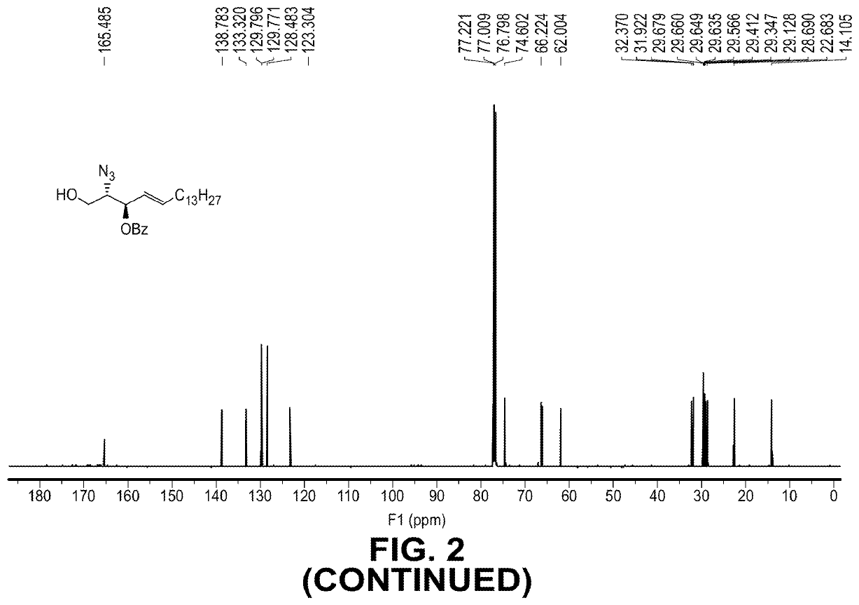

The freshly prepared solution of TfN₃ (1.5 equivalents (equiv. or eq.), 5.6 mmol in 5 mL CH₂Cl₂) was added to a mixture of compound I-5 (1.5 g, 3.7 mmol), Et₃N (0.78 mL, 5.6 mmol), and CuSO₄ (10 mg). Methanol (MeOH) (1 mL) was then added dropwise and the resulting solution was stirred at room temperature overnight. The volume of the reaction mixture was reduced and the resulting residue was purified by silica gel column chromatography using hexane:EtOAc=4:1 (by volume) as an eluant to obtain compound I-6 (1.37 g, 86%) as a white solid. ¹H NMR (600 MHz, CDCl₃) δ 8.07-8.05 (m, 2H), 7.60-7.57 (m, 1H), 7.45 (t, J=7.8 Hz, 2H), 5.96 (dt, J=13.8, 6.6 Hz, 1H), 5.64-5.58 (m, 2H), 3.82-3.79 (m, 1H), 3.76 (dd, J=10.8, 4.2 Hz, 1H), 3.64 (dd, J=11.4, 7.2 Hz, 1H), 2.11-2.04 (m, 3H), 1.41-1.36 (m, 2H), 1.31-1.20 (m, 22H), 0.88 (t, J=7.2 Hz, 3H). ¹³C NMR (150 MHz, CDCl₃) δ 165.48, 138.78, 133.32, 129.80, 129.77, 128.48, 123.30, 74.60, 66.22, 62.00, 32.37, 31.92, 29.68, 29.66, 29.65, 29.63, 29.57, 29.41, 29.35, 29.13, 28.69, 22.68, 14.10. HRMS (ESI-Orbitrap) m/z: [M+Na]⁺ calculated for C₂₅H₃₉N₃O₃Na 452.2889, found 452.2875. See FIG. 2 for ¹H and ¹³C NMR spectra of compound I-6.

### O-(2,3,4,6-Tetra-O-benzoyl-β-D-galactopyranosyl)-(1→4)-(2,3,6-tri-O-benzoyl-α-D-glucopyranosyl)-(1→1)-(2S,3R,E)-2-azido-3-O-benzoyloxy-octadec-4-ene (I-8)

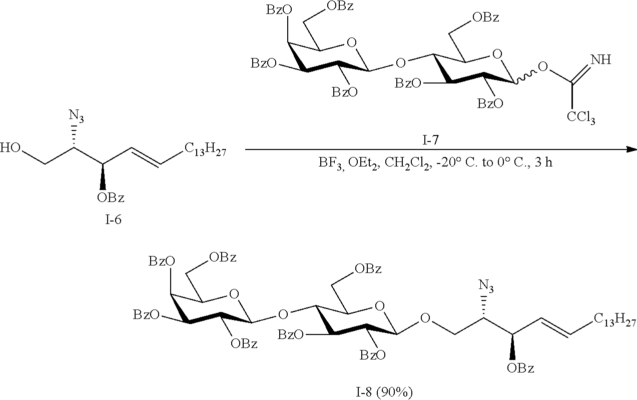

Perbenzoylated lactosyl trichloroacetimidate (I-7, 3.48 g, 2.87 mmol) and glycosyl acceptor I-6 (0.82 g, 1.9 mmol) were dissolved in anhydrous CH₂Cl₂ (80 mL) containing powdered molecular sieves (4 Å, 2.0 g). The mixture was stirred under argon at room temperature for 30 min and then cooled down to −20° C. BF₃·OEt₂ (0.77 mL, 6.3 mmol) was added and the reaction mixture was stirred at −20° C. for 30 minutes. The temperature of the reaction was then slowly increased to 0° C. and the mixture was continuously stirred at 0° C. (by incubating the reaction container in an ice-water bath) until TLC analysis (hexane:EtOAc=3:1 by volume and detected with p-anisaldehyde sugar stain) showed the complete consumption of the acceptor (3 hours). The reaction was quenched with Et₃N, and the mixture was filtered over Celite and concentrated. The residue was purified by silica gel column chromatography using toluene:EtOAc=20:1 (by volume) as an eluant to obtain compound I-8 (2.55 g, 90%) as a white foam. ¹H NMR (600 MHz, CDCl₃) δ 8.04-7.16 (m, 40H), 5.81 (t, J=9.6 Hz, 1H), 5.75-5.65 (m, 3H), 5.51-5.47 (m, 2H), 5.45-5.35 (m, 2H), 4.89 (d, J=7.8 Hz, 1H), 4.74 (d, J=7.8 Hz, 1H), 4.58 (d, J=12.0 Hz, 1H), 4.48 (dd, J=12.0, 4.8 Hz, 1H), 4.29 (t, J=9.6 Hz, 1H), 3.92-3.84 (m, 4H), 3.77-3.69 (m, 2H), 3.55 (dd, J=10.2, 6.0 Hz, 1H), 1.89 (q, J=7.2 Hz, 2H), 1.37-1.05 (m, 22H), 0.88 (d, J=7.2 Hz, 3H). ¹³C NMR (150 MHz, CDCl₃) δ 165.80, 165.56, 165.40, 165.22, 165.02, 164.93, 164.81, 138.95, 133.54, 133.42, 133.38, 133.34, 133.26, 133.22, 133.20, 133.05, 130.00, 129.93, 129.87, 129.76, 129.71, 129.66, 129.58, 129.54, 129.44, 129.41, 129.29, 128.97, 128.87, 128.68, 128.64, 128.58, 128.55, 128.51, 128.38, 128.36, 128.28, 128.25, 122.41, 101.03, 100.83, 75.87, 74.78, 73.12, 72.87, 71.78, 71.65, 71.43, 69.89, 68.28, 67.54, 63.42, 62.27, 61.07, 32.28, 31.94, 29.71, 29.68, 29.65, 29.59, 29.37, 29.14, 28.60, 22.71, 14.15. HRMS (ESI-Orbitrap) m/z: [M+Na]⁺ calculated for C₈₆H₈₇N₃O₂₀Na 1504.5781, found 1504.5782.

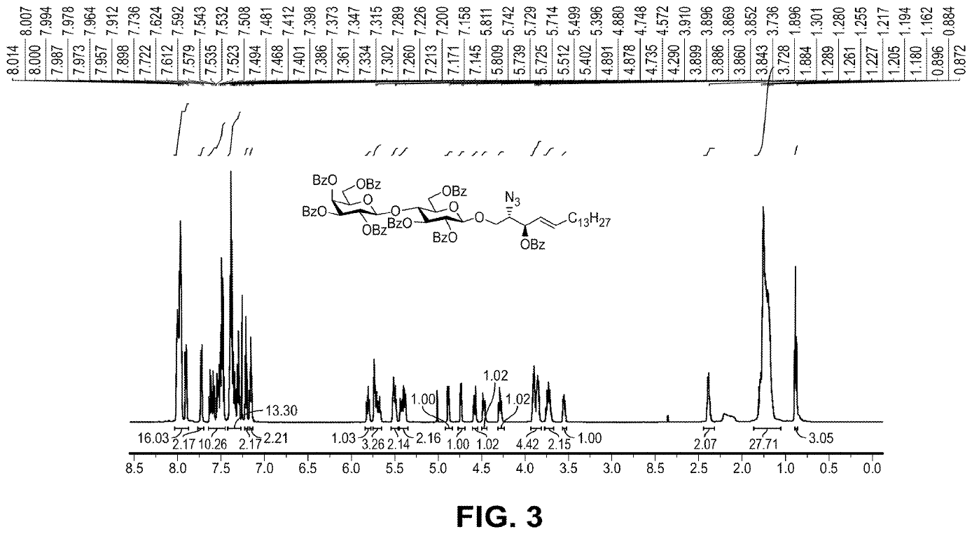

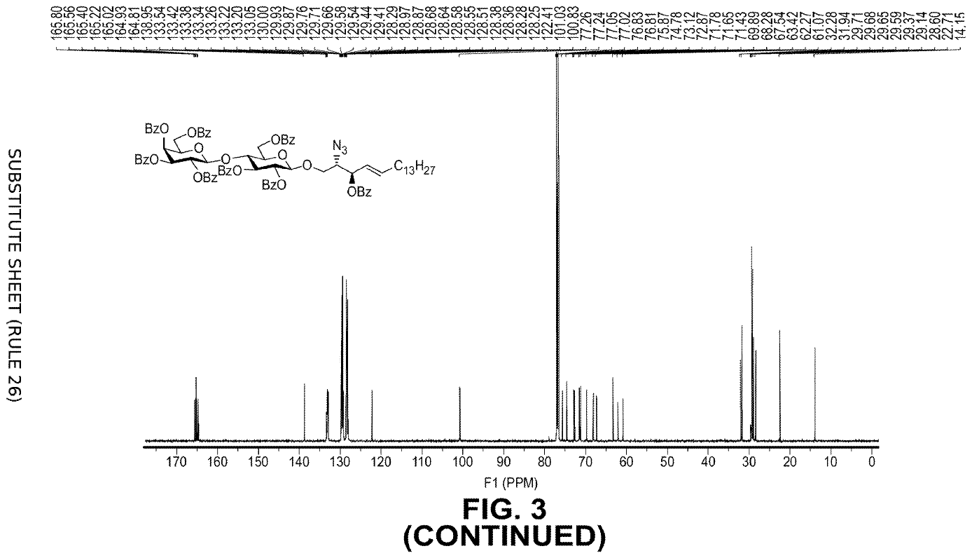

See FIG. 3 for ¹H and ¹³C NMR spectra of compound I-8.

### O-(β-D-Galactopyranosyl)-(1→4)-(β-D-glucopyranosyl)-(1→1)-(2S, 3R, E)-2-aminooctadec-4-ene-1,3-diol (LacβSph, I-9)

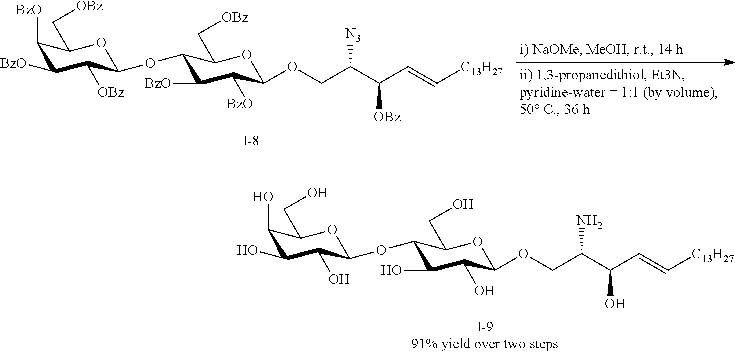

To a solution of I-8 (2.50 g, 1.7 mmol) in dry MeOH (30 mL), NaOMe (250 mg) was added. After being stirred at room temperature for 14 hours, the reaction mixture was neutralized using Dowex® 50W (H⁺), filtered and concentrated under a reduced pressure.

The intermediate was used in the next step without further purification. To the dry intermediate in pyridine-water=1:1 (by volume, 30 mL), 1,3-propanedithiol (1.83 mL, 16.8 mmol) and Et₃N (0.43 mL) were added, and the mixture was stirred at 50° C. for 36 hours.

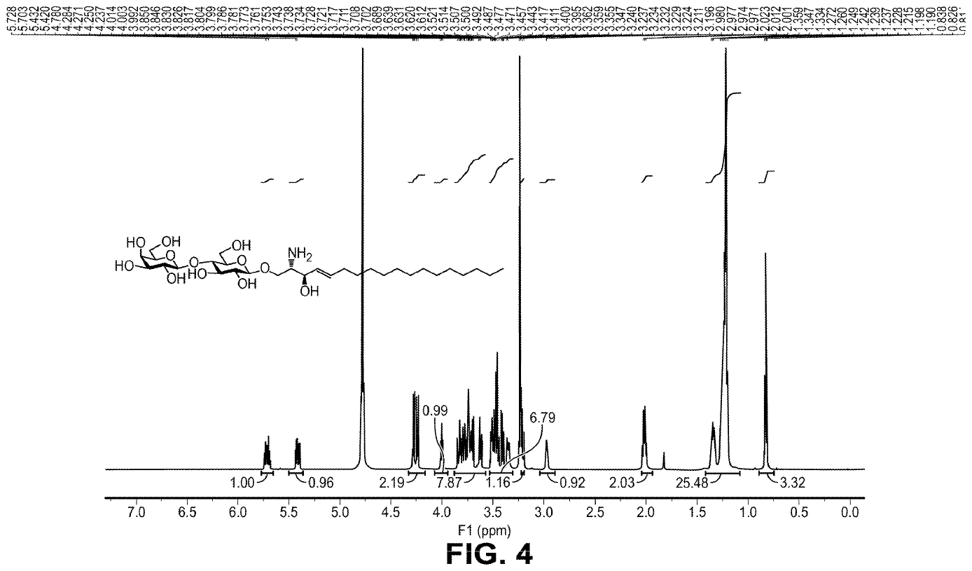

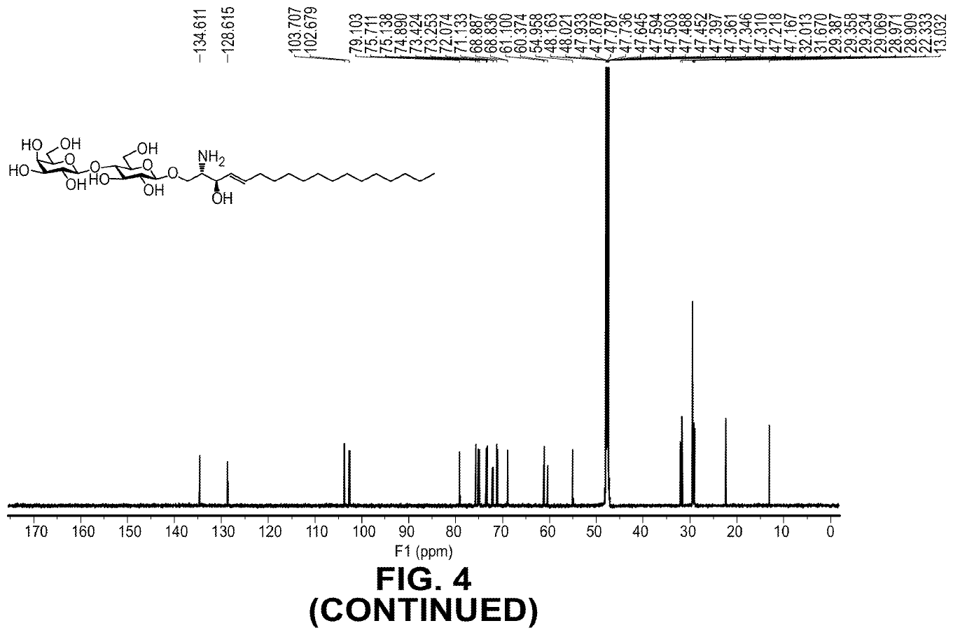

The reaction mixture was concentrated and purified by silica gel column chromatography using chloroform:imethanol:water=5:4:0.5 (by volume) as an eluant to obtain LacPSph (I-9, 1.05 g, 91%) as a white powder. ¹H NMR (600 MHz, CD₃OD) δ 5.72 (dt, J=14.4, 6.6 Hz, 1H), 5.41 (dd, J=15.6, 7.2 Hz, 1H), 4.28 (d, J=7.8 Hz, 1H), 4.24 (d, J=7.8 Hz, 1H), 4.00 (t, J=6.6 Hz, 1H), 3.84 (dd, J=12.0, 2.4 Hz, 1H), 3.83-3.69 (m, 5H), 3.62 (dd, J=11.4, 4.8 Hz, 1H), 3.52-3.44 (m, 4H), 3.41 (dd, J=10.2, 3.6 Hz, 1H), 3.37-3.34 (m, 1H), 3.21 (t, J=8.4 Hz, 1H), 2.98 (ddd, J=7.8, 6.0, 3.6 Hz, 1H), 2.04-1.94 (m, 2H), 1.42-1.08 (m, 20H), 0.83 (t, J=7.2 Hz, 3H). ¹³C NMR (150 MHz, CD₃OD) δ 134.61, 128.61, 103.71, 102.68, 79.10, 75.71, 75.14, 74.89, 73.42, 73.25, 72.07, 71.13, 68.89, 68.84, 61.10, 60.37, 54.96, 32.01, 31.67, 29.39, 29.36, 29.23, 29.07, 28.97, 28.91, 22.33, 13.03. HRMS (ESI-Orbitrap) m/z: [M+H]⁺ calculated for C₃₀H₅₈NO₁₂ 624.3959, found: 624.3950. See FIG. 4 for ¹H and ¹³C NMR spectra of compound I-9.

### Example 2: Cost-Effective Chemical Synthesis of Lactosylsphingosine and Other Simple Glycosphingosines

A highly efficient chemoenzymatic total synthesis strategy to access a diverse array of structurally defined glycosphingolipids (GSLs) is provided herein. The obtained GSLs are valuable standards for research as well as for analysis and quality control of GSL production, and also have therapeutic uses.

The chemoenzymatic strategy for the total synthesis of complex GSLs starts with chemical synthesis of lactosylsphingosine (LacβSph), which is used as the acceptor substrate for a glycosyltransferase in a one-pot multienzyme (OPME) system to extend the glycan structure. Multiple OPME reactions performed in sequence lead to the formation of the desired complex glycosphingosine intermediates. A final acylation reaction produces the final glycosphingolipid targets. Both the sphingosine and the ceramide can serve as the hydrophobic tag to facilitate the purification of glycosphingosine intermediates and glycosphingolipid targets by a single C18-cartridge in less than 30 minutes.

Different routes can access lactosylsphingosine (LacβSph), the key intermediate for glycosyltransferase-based enzymatic extension of the glycan chains of complex glycosphingosines. Three of these routes are described below, and are referred to as the first generation, the second generation, and the third generation syntheses. A first generation synthesis started from phytosphingosine and involved eight steps with an overall 70% yield to produce the 2-azido 3-O-benzoyl derivative of sphingosine as a well suited glycosyl acceptor for chemical glycosylation. Phytosphingosine ($272 for 5 g from TCI Chemical; Portland. OR) is much less expensive than D-erythro-sphingosine (d18:1) ($1094 for 250 mg from Fisher Scientific). However, its cost is still relatively high and five silica gel column purification steps are required. In addition, it is more suitable for the synthesis of targets with a d18:1 sphingosine component, but is not ideal for those with a d20:1 sphingosine. The second generation of sphingosine acceptor synthesis used a less expensive partially protected L-serine derivative, N-Boc L-serine methyl ester ($44 for 100 g from Aaron Chemicals; San Diego, CA), and tetradecanal ($329/g from TCI America) as the starting materials to produce a differently protected sphingosine glycosylation acceptor in eight steps with an overall 30% yield with two silica gel column purification steps and one crystallization process. The method was demonstrated for the synthesis of d18:1 sphingosine glycosyl acceptor but is also applicable to access the d20:1 sphingosine. The third generation synthesis used (S)-Gamer's aldehyde ($142 for 1 g from Fisher Scientific) as a starting material to prepare the 2-azido 3-O-benzoyl derivative of sphingosine in a short four-step synthesis with an overall 49% yield.

To further decrease the cost for large-scale production, described herein is the presently claimed method (also referred to herein as the “fourth generation synthesis”) of synthesizing a variety of glycosyl acceptors, including both the d18:1 and d20:1 D-erythro-sphingosine glycosyl acceptors.

A chiral pool approach, using low-cost renewable monosaccharides with the desired stereocenters as starting materials, was used. D-Xylose ($105 for 500 g from Thermo Scientific) was identified as the starting material. A cost-effective chemical synthesis of lactosylsphingosine and other simple glycosphingosines is provided herein, as shown below in Schemes 2-8).

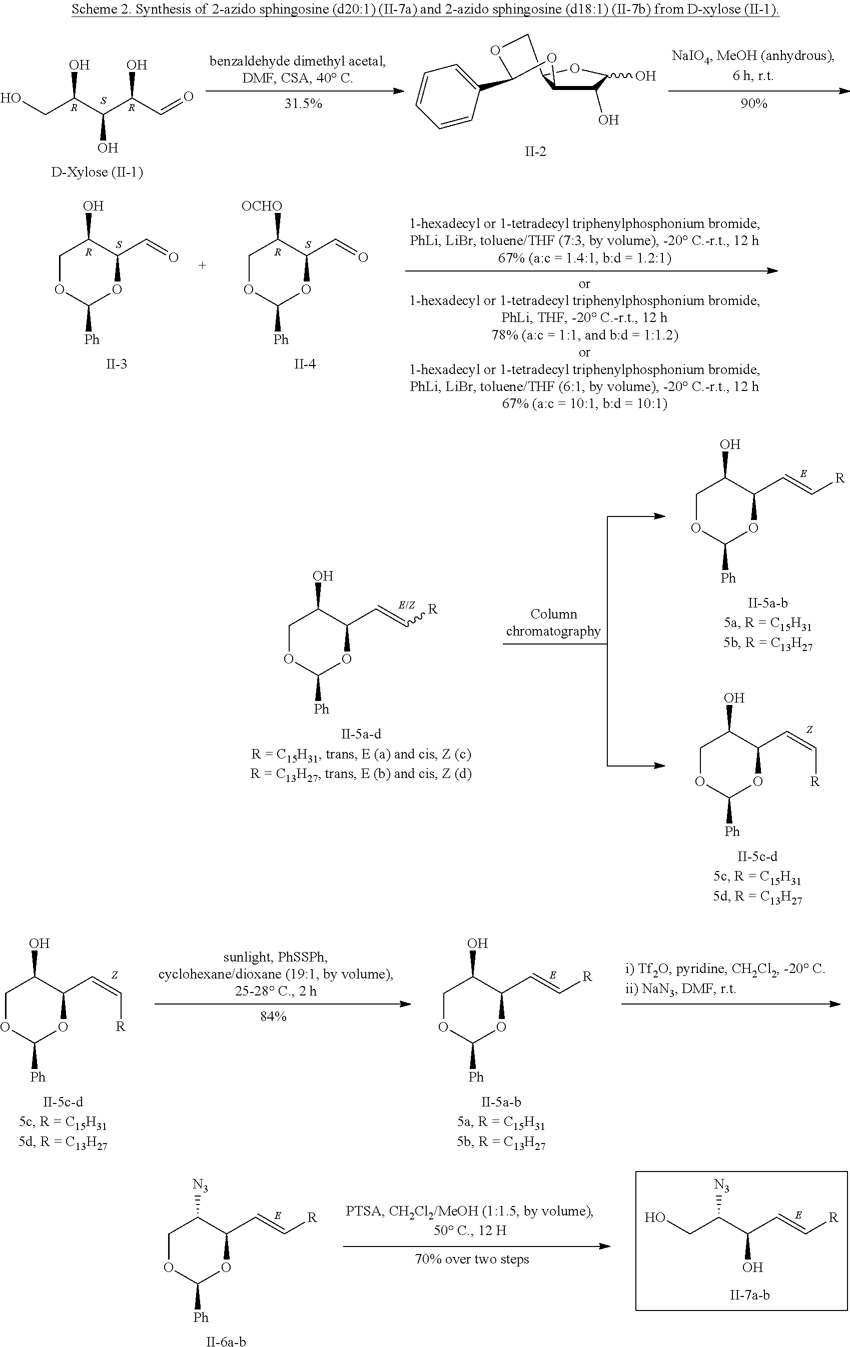

As shown in Scheme 2, a one-step treatment of D-xylose (II-1) with benzaldehyde dimethyl acetal and camphor-10-sulfonic acid in N,N-dimethylformamide (DMF) at 40° C. produced 3,5-O-benzylidene-D-xylofuranose (II-2) in 31.5% yield. It was then subjected to periodate oxidative cleavage by treating with sodium metaperiodate in methanol to form a mixture of 2,4-O-benzylidene-D-threose (II-3) and its formate (II-4) in 90% yield. Compound II-4 was readily converted to compound II-3 under the Wittig reaction conditions in the next step. These intermediates can also be obtained from 4,6-O-benzylidene-D-galactopyranose by periodate oxidative cleavage but, unexpectedly, the present method provided better yields and the reactions went more smoothly as only one C—C bond cleavage was involved compared to the cleavage of two C—C bonds in the latter method. The periodate oxidation of 3,5-O-benzylidene-D-xylofuranose is pH independent, while the oxidation of 4,6-O-benzylidene-D-galactopyranose is pH (7.6) dependent which further complicates the reaction process.

A major advantage of preparing 3,5-O-benzylidene-D-xylofuranose (II-2), instead of other reagents (e.g., 3,5-O-isopropylidene-D-xylofuranose) is easier product purification due to the formation of less 1,2-protected byproduct in the former. For example, 1,2-O-benzylidene-D-xylopyranose (<5%) formed in the former was much less compared to 1,2-O-isopropylidene-D-xylopyranose (16%) formed in the latter, as the isopropylidene favors the formation of five membered rings and benzylidene favors the formation of six-membered rings.

The Wittig alkenylation reactions of 2,4-O-benzilidene-D-threose (II-3) with 1-hexadecyltriphenylphosphonium bromide (prepared from PPh₃ and alkyl bromide, or commercially available from Thermo Scientific) or 1-tetradecyltriphenylphosphonium bromide (prepared from PPh₃ and alkyl bromide, or commercially available from Thermo Scientific) with phenyllithium in tetrahydrofuran (THF) with or without toluene and in the presence or the absence of LiBr were carried out. The product yield of the Wittig alkenylation reactions in the presence of LiBr with a mixed solvent of toluene/THF was 67% with the E isomer (II-5a or II-5b) as the predominant product. The ratio of toluene and THF in the solvent affected the ratio of the E:Z isomers in the products. Under these conditions, when toluene/THF (7:3, by volume) was used as the solvent, the E:Z ratios of the products were 1.4:1 for II-5a:II-5c and 1.2:1 for II-5b:II-5d; in comparison when toluene/THF (6:1, by volume) was used as the solvent, the E:Z ratio of the products was 10:1 for both II-5a:II-5c and II-5b:II-5d. In the absence of LiBr with THF as the solvent, the Z isomer was the predominant product (II-5d) or was obtained in the equal concentration (II-5c) as the E isomer. The E isomer and Z isomers were purified from the reaction mixture to obtain the pure F isomer as the target compound and the pure Z isomer. The pure Z isomer (II-5c or 11-5d) was converted readily to the corresponding E isomer (II-5a or II-5b) via a photo-isomerization reaction for 2 hours using sunlight as the light source and the E isomer which was purified with an 84% yield for 1-10 g-scale reactions. Using a metal halide lamp (250 W or 400 W) resembling the reported use of a mercury lamp as the light source was similarly effective as the sunlight when a Pyrex glass container was used for the reaction (See Table 1). Non-Pyrex glass containers were not as good.

TABLE 1
Photo-isomerization reaction conditions and yields comparison.

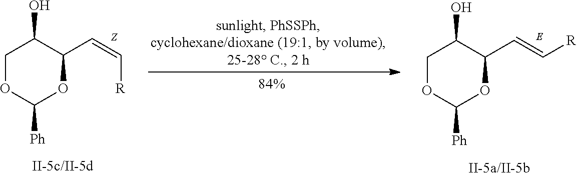

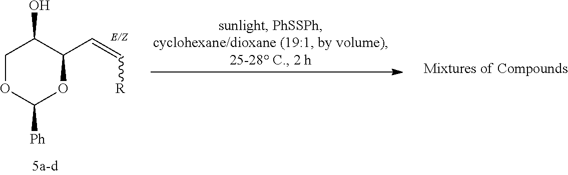

R = C₁₅H₃₁, trans, E (a) and cis, Z (c)
R = C₁₃H₂₇, trans, E (b) and cis, Z (d)

| | | | | | | | Isolated |
|---|---|---|---|---|---|---|---|
| | | Glass | Light | Amount | | Conversion^{a} | Yield |
| Entry | Isomer | container | (Watts) | (g) | Time | (%) | (%) |
| 1 | Z | Non-Pyrex | MH 250 | 1.0 | 2 h | 50 | 45 |
| 2 | Z | Non- Pyrex | MH 250 | 1.0 | 8 h | 50 | 47 |
| 3 | Z | Pyrex | MH 400 | 1.0 | 2 h | >95 | 87 |
| 4 | Z | Pyrex | MH 400 | 5.0 | 3 h | 90 | 80 |
| 5 | Z | Non- Pyrex | MH 400 | 10.0 | 3 h | 50 | NA |
| 6 | Z | Pyrex | MH 400 | 10.0 | 8 h | 30 | 20 |
| 7 | Z | Pyrex | MH 600 | 1.0 | 1 h | >90 | 80 |
| 8 | Z | Pyrex | MH 600 | 5.0 | 1 h | 90 | 75 |
| 9 | Z | Pyrex | MH 600 | 5.0 | 3 h | 40 | NA |
| 10 | Z | Pyrex | Sunlight | 1.0 | 10 | >30 | NA |
| | | | | | min | | |
| 11 | Z | Pyrex | Sunlight | 1.0 | 20 | >40 | NA |
| | | | | | min | | |
| 12 | Z | Pyrex | Sunlight | 1.0 | 30 | >50 | NA |
| | | | | | min | | |
| 13 | Z | Pyrex | Sunlight | 1.0 | 1 h | >70 | NA |
| 14 | Z | Pyrex | Sunlight | 1.0 | 1.5 h | >70 | NA |
| 15 | Z | Pyrex | Sunlight | 1.0 | 2 h | >80 | 84 |
| 14 | Z | Pyrex | Sunlight | 1.0 | 3 h | >80 | 82 |
| 16 | Z | Pyrex | Sunlight | 5.0 | 2 h | >80 | 82 |
| 17^{b} | Z | Pyrex | Sunlight | 10.0 | 2 h | >80 | 83 |
| 18 | E + Z | Pyrex | MH 400 | 1.0 | 1 h | not | NA |
| | (E:Z = | | | | | determined | |
| | 1:1) | | | | | (Mixture) | |
| 19 | E + Z | Pyrex | MH 600 | 5.0 | 1 h | >60 | 56 |
| | (E:Z = | | | | | | |
| | 1:8) | | | | | | |
| 20 | E + Z | Pyrex | Sunlight | 1.0 | 2 h | >80 | 83 |
| | (E:Z = | | | | | | |
| | 1:8) | | | | | | |
| 21 | Z | Pyrex | MH 250 | 1.0 | 2 h | 50 | 45 |
| 22 | Z | Pyrex | MH 250 | 1.0 | 6 h | >90 | 84 |
| 23 | Z | Pyrex | MH 200 | 1.0 | 8 h | >90 | 84 |
| 24 | E + Z | Pyrex | MH 400 | 1.0 | 2 h | not | NA |
| | (E:Z = | | | | | determined | |
| | 1:1.2) | | | | | (Mixture) | |
| 25 | E + Z | Pyrex | MH 600 | 1.0 | 1 h | not | NA |
| | (E:Z = | | | | | determined | |
| | 1:1.2) | | | | | (Mixture) | |
| 26 | E + Z | Pyrex | Sunlight | 1.0 | 2 h | not | NA |
| | (E:Z = | | | | | determined | |
| | 1:1.2) | | | | | (Mixture) | |

ªEstimated by TLC Only; all compounds tested were Sph-d18:1 analogues except for ^{b}Sph-d20:1 analogue. Abbreviations: MH, metal halide lamp; NA, not applicable.

The 1,3-di-O-benzylidene protected olefin (E) (TT-5a or II-5b) was treated with Tf₂O and pyridine in CH₂Cl₂, and then subjected to an in-situ S_{N}² replacement reaction with sodium-azide in DMF to form the corresponding 1,3-di-O-benzylidene-protected 2-azido-sphingosine (II-6a or II-6b). Hydrolysis using PTSA in CH₂Cl₂/MeOH at 50° C. produced the desired 2-azido sphingosine (II-7a or II-7b) in 70% yield over two steps as an acceptor for chemical glycosylation reactions.

The 3-OH of 2-azido sphingosine (II-7a or II-7b) was protected by O-benzoyl to provide an alternative glycosyl acceptor 2-azido 3-O-benzoyl sphingosine (d20:1) (II-8a) or (d18:1) (II-8b) via a three-step procedure by treating with TBDPSCl and Et₃N in CH₂Cl₂/DMF followed by the addition of benzoyl chloride and finally the removal of TBDPS by HF/pyridine (Scheme 3). An overall yield of 85% was achieved.

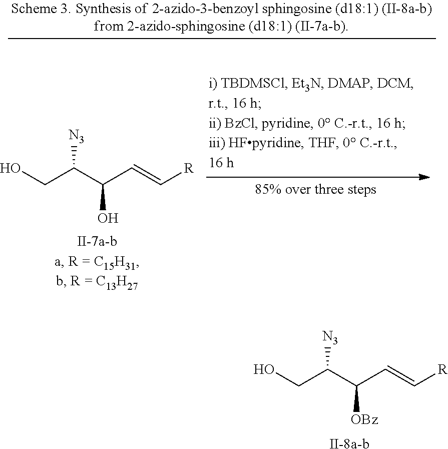

An acceptable 65% yield was achieved for glycosylating compound II-7b with per-O-benzyl lactosyl trichloroacetimidate donor II-9 (Scheme 4) although it was lower than that (90% yield) for glycosylating compound II-8b (Scheme 5).

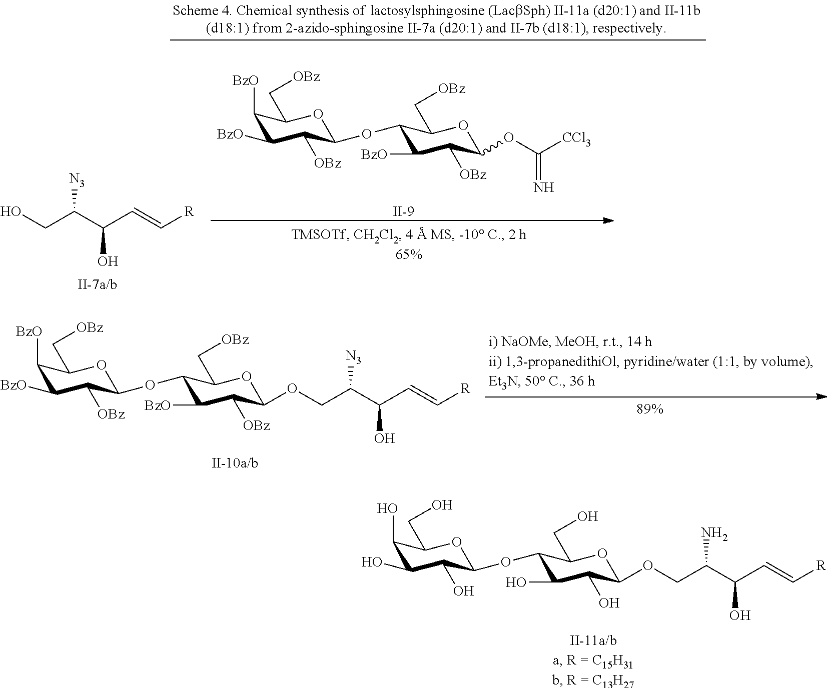

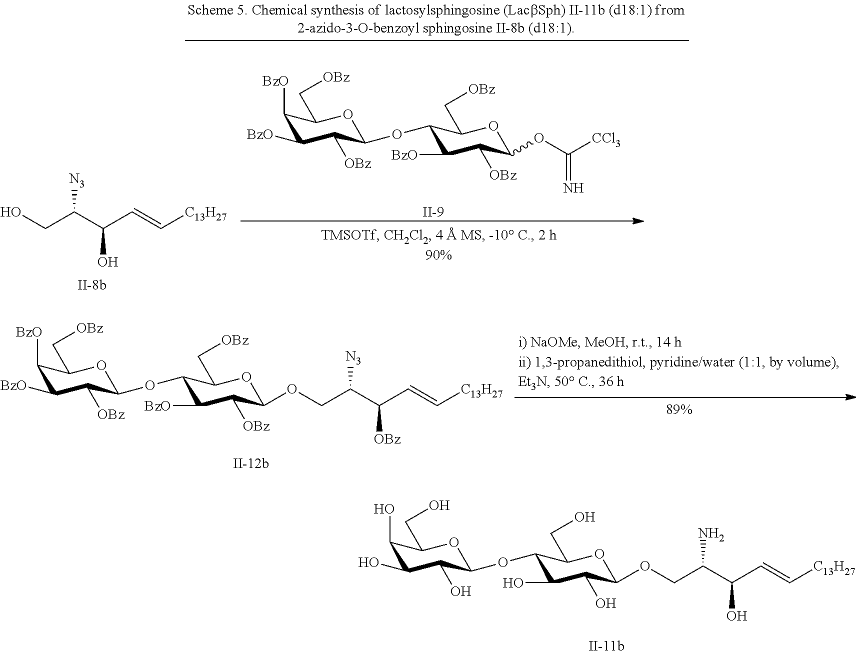

Compounds II-7a and II-7b were used for synthesizing glycosphingosines at −10° C. in CH₂Cl₂ using various trichloroacetimidate glycosyl donors and TMSOTf as a promoter (Schemes 4, 6-7). De-O-benzoylation of the glycosyl products using NaOMe in MeOH and conversion of N₃ to N₁₂ using 1,3-propane-di-thiol in H₂O/pyridine at 50° C. produced the corresponding glycosyl sphingosines. Glycosylation yields were in the range of 55-65% for lactose (65%), glucose (57%), and galactose (55%); and deprotection yields were in the range of 85-89%.

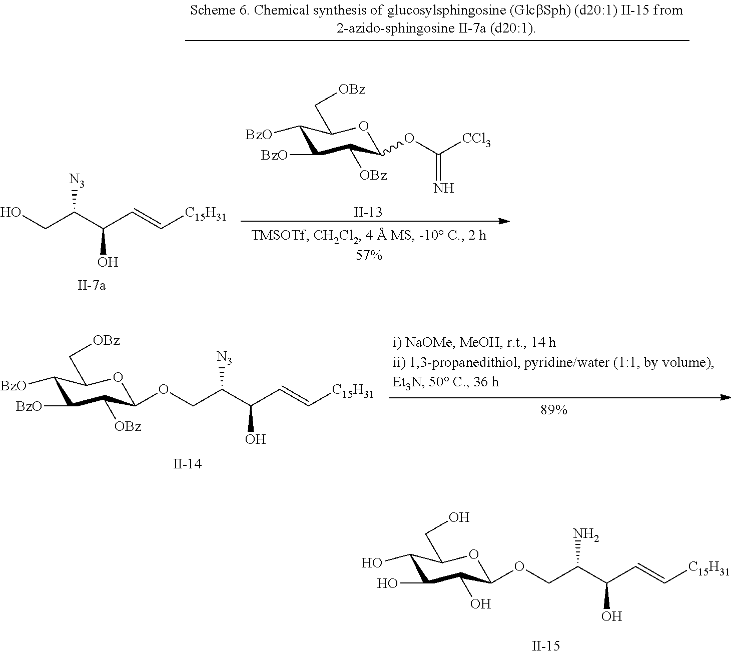

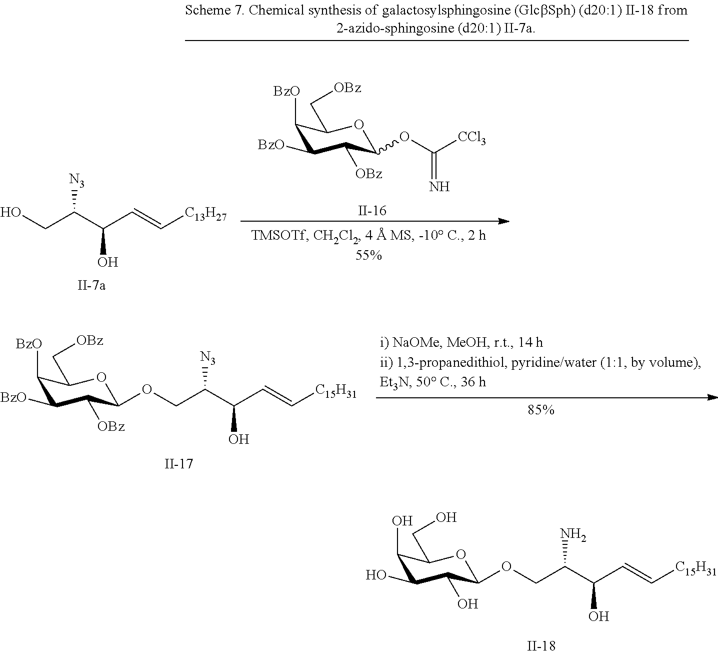

Sulfatide sphingosine (d20:1) (II-21) was also successfully synthesized in 75% yield by selectively sulfation of the 3-OH of the galactose in galactosyl azido-sphingosine (d20:1) (II-19) followed by converting the azido group to an amino group in 89% yield (Scheme 8).

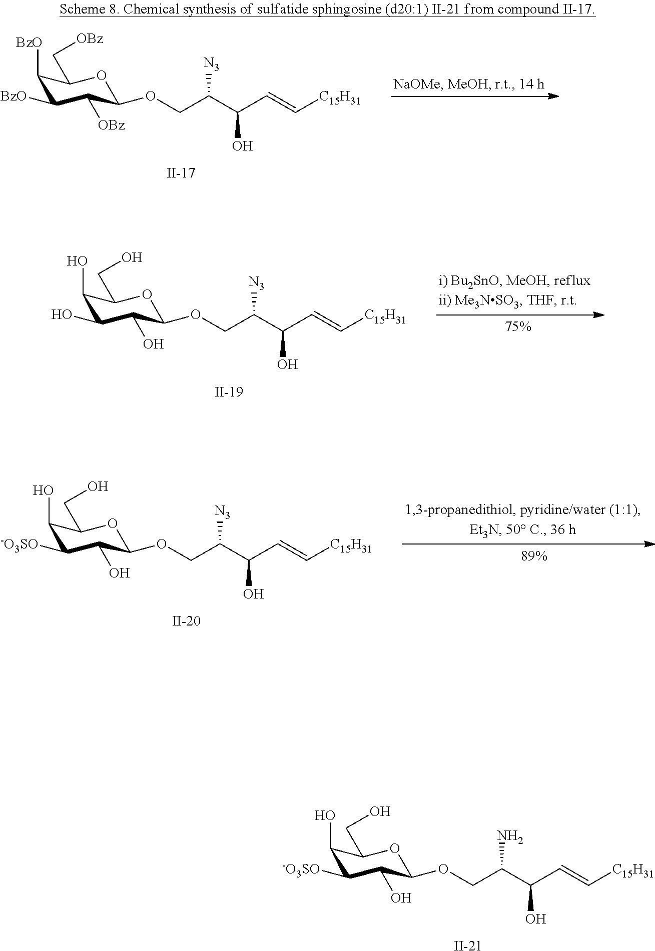

### General Methods

Chemicals were purchased and used without further purification. ¹H NMR and ¹³C NMR spectra were recorded on 300 MHz Brucker Avance III and 400 MHz Brucker Avance III spectrometers. High-resolution electrospray ionization (ESI) mass spectra were recorded using a Thermo Scientific Q Exactive HF Orbitrap Mass Spectrometer. Silica gel 60 Å (230-400 mesh, Sorbent Technologies) was used for flash column chromatography. Thin-layer chromatography (TLC, Sorbent Technologies) was performed on silica gel plates using anisaldehyde sugar staining or 5% sulfuric acid in ethanol staining for detection.

### Chemical synthesis of [2(S), 3(R), 4E]-2-azido-icos-4-ene-1,3-diol (7a) and [2(S), 3(R), 4E]-2-azido-octadec-4-ene-1,3-diol (II-7b)

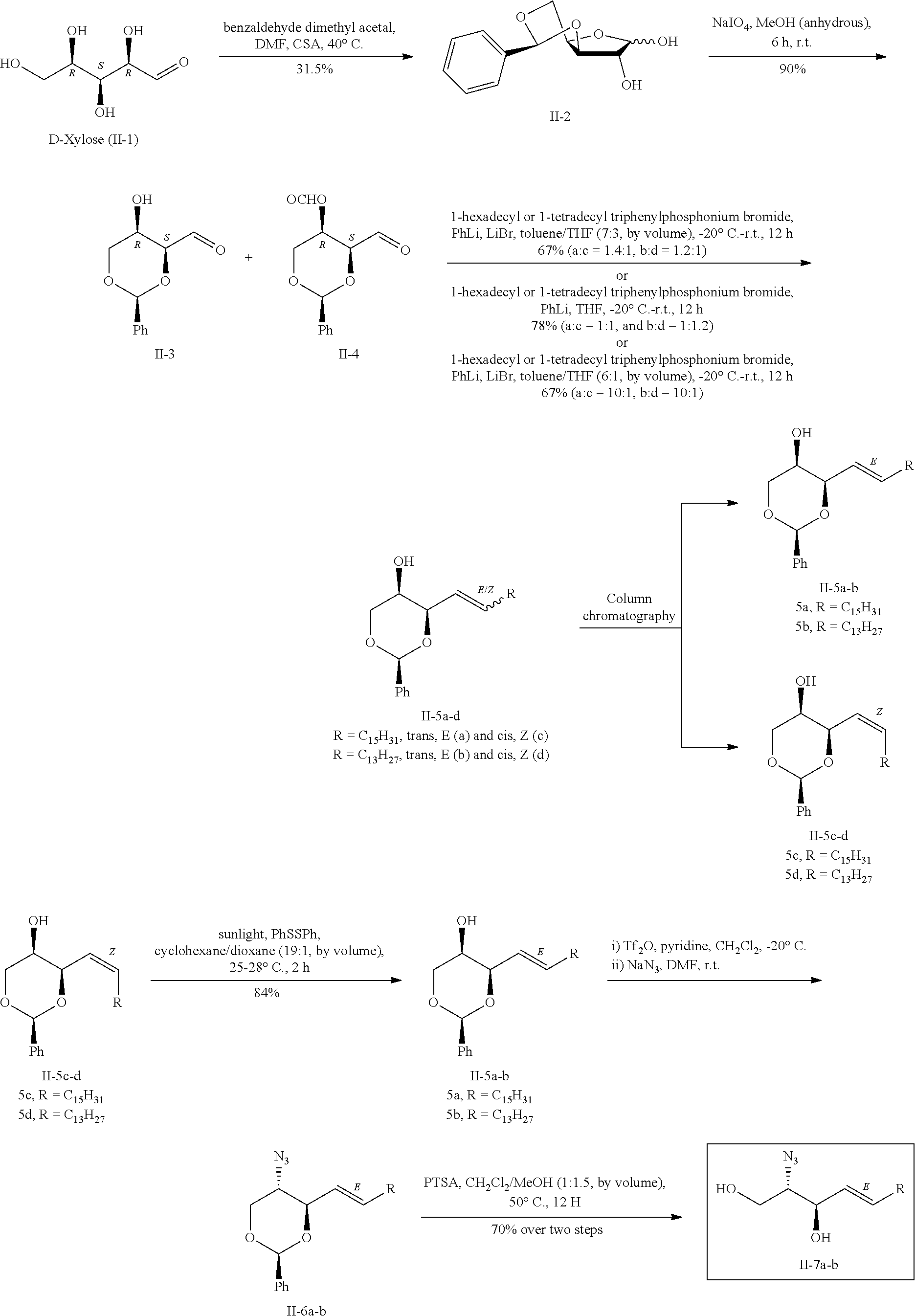

### 3,5-O-Benzylidene-D-xylofuranose (II-2)

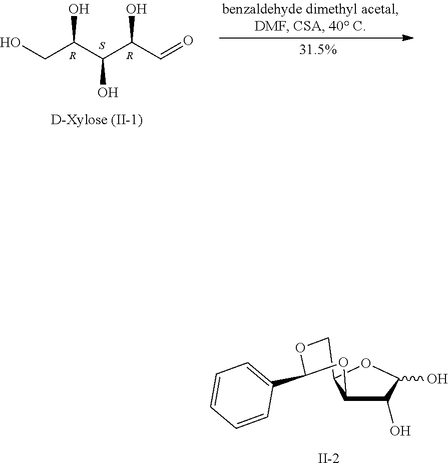

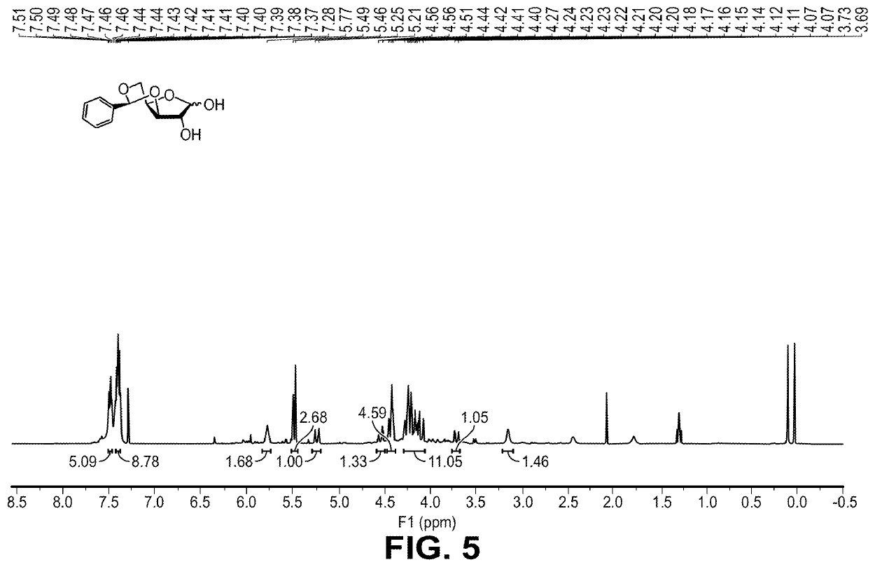

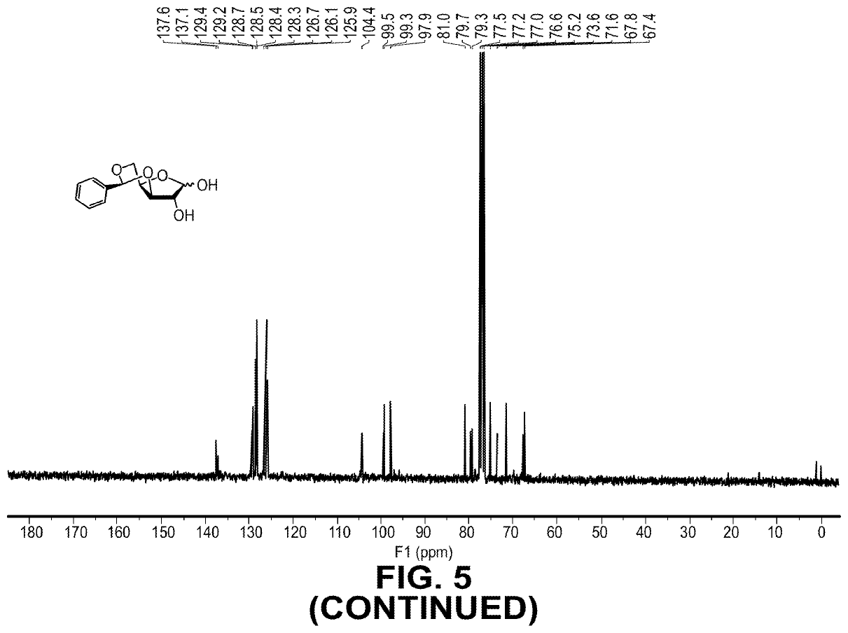

To a solution of D-xylose (II-1) (4 g, 26.6 mmol) and benzaldehyde dimethyl acetal (10 mL, 66.6 mmol) in 50 mL of DMF, camphor-10-sulfonic acid (100 mg, 0.43 mmol) was added and the reaction mixture was stirred at 40° C. for 8-12 hours. The reaction mixture was then neutralized by adding Et₃N and the solvent was evaporated. The crude was washed with hexane (100 mL). The dissolved component was discarded and the undissolved part was purified by a silica gel column chromatography using hexane:EtOAc=2:3 (by volume) as an eluant to produce pure 3,5-O-benzylidene-D-xylofuranose (II-2) as a white solid (2 g, 8.4 mmol). The reaction was repeated several times from 8 g. 25 g. and 50 g of D-xylose. The reaction yields were in the range of 28-31.5%. ¹H NMR (300 MHz, CDCl₃) δ 7.50-7.46 (dt, J=5.3, 2.2 Hz, 5H), 7.42-7.37 (td, J=5.0, 4.6, 2.9 Hz, 9H), 5.83-5.73 (s, 2H), 5.51-5.44 (d, J=7.4 Hz, 3H), 5.28-5.19 (d, J=12.3 Hz, 1H), 4.58-4.49 (m, 1H), 4.47-4.38 (m, 5H), 4.29-4.06 (m, 11H), 3.76-3.67 (d, J=12.5 Hz, 1H), 3.21-3.09 (s, 1H); ¹³C NMR (75 MHz, CDCl₃) δ 137.6, 137.1, 129.4, 129.2, 128.7, 128.5, 128.4, 128.3, 126.7, 126.1, 125.9, 104.4, 99.5, 99.3, 97.9, 81.0, 79.7, 79.3, 77.5, 77.2, 77.0, 76.6, 75.2, 73.6, 71.6, 67.8, 67.4. See FIG. 5 for ¹H and ¹³C NMR spectra of compound H₁-2.

### 2,4-O-Benzylidene-D-threose (3) and its formate (II-4)

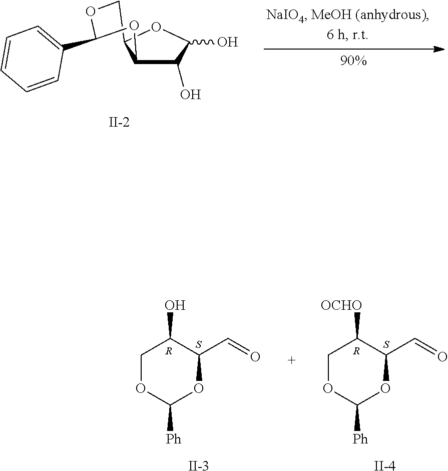

To a solution of 3,5-O-benzylidene-D-xylofuranose (II-2) (15 g, 63.0 mmol, 1 eq.) in 500 mL/g of MeOH (anhydrous), sodium metaperiodate (22.5 g, 105.2 mmol, 1.67 eq.) was added and the mixture was stirred at room temperature for 6 hours. The resulting precipitation was filtered off using Celite and washed with MeOH. The filtrate and the MeOH washing were combined and evaporated to dryness. The obtained crude was dissolved in CH₂Cl₂ using a sonicator and further filtered with a new Celite bed, washed with CH₂Cl₂ and then with 5% CH₃OH in CH₂Cl₂. The filtrate was evaporated to dryness to obtain the mixture of 2,4-O-benzylidene-D-threose (3) and its formate (II-4) (14.4 g, 90%). The obtained residue was used for next reaction without further purification.

### (1-Tetradecyl) Triphenylphosphoniumbromide and (1-Hexadecyl) Triphenylphosphoniumbromide

A solution of PPh₃ (95 g. 360.0 mmol) in bromotetradecane (107.5 mL, 360.0 mmol) or bromohexadecane (110 mL, 360.0 mmol) was heated to 140° C. under dry conditions, and the mixture was stirred for 5 hours. The solution was cooled to approximately 40° C. and the product was crystallized by adding 500 mL of dry acetone followed by 2 L of dry diethyl ether to obtain (1-tetradecyl) triphenylphosphoniumbromide (155 g, 78%) or (1-hexadecyl) triphenylphosphoniumbromide (160 g, 78%) as white crystals.

### 1,3-O-Benzylidene-4-icosene-2-ol (II-5a and II-5c)

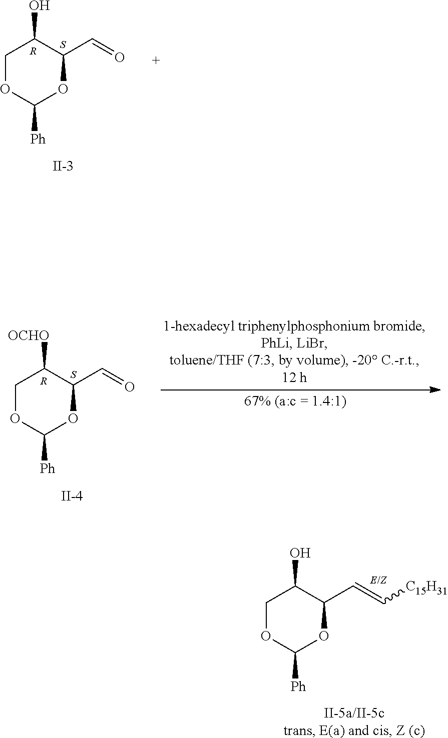

To a solution of (1-hexadecyl) triphenylphosphonium bromide (39.0 g, 68.8 mmol, 1.25 eq.) and LiBr (14.3 g, 165.0 mmol, 3 eq.) in toluene:THF=7:3 (by volume) (250 mL), 1.9 M PhLi in dibutyl ether solution (87 mL, 165.0 mmol, 3 eq.) was added at 0° C. and the reaction mixture was stirred at room temperature for 30 min. The resulting mixture was cooled to −20° C. A solution of 2,4-O-benzylidene-D-threose (II-3) and its formate (II-4) (14 g, 55.0 mmol) in THF (70 mL) was added. The resulting mixture was stirred at the same temperature (−20° C.) for 2 hours and then at room temperature for another 11 hours. Water was then added to the reaction mixture. The products were extracted with EtOAc, dried over Na₂SO₄, and the mixture was evaporated to dryness. The obtained crude was purified by a silica gel column chromatography using hexane:EtOAc=4:1 (by volume) as an eluant to obtain 1,3-O-benzylidene-4-icosene-2-ol as a mixture of E (II-5a) and Z (II-5c) isomers (E:Z=1.4:1) (16.3 g) in 67% yield.

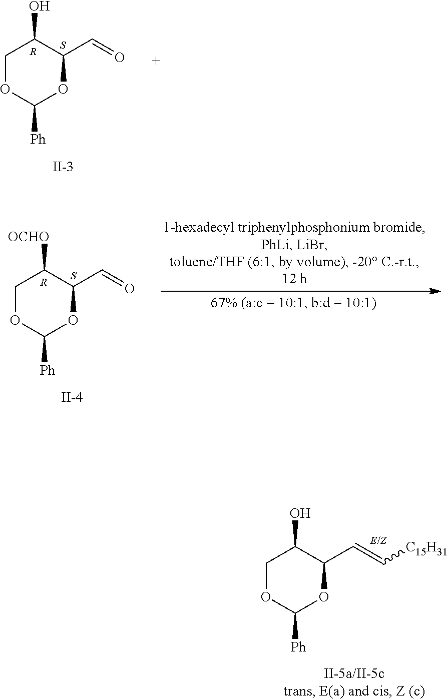

Example i): To a solution of (1-hexadecyl) triphenylphosphonium bromide (2.8 g, 4.9 mmol, 1.25 eq.) and LiBr (1.36 g, 15.7 mmol, 4 eq.) in toluene (42 mL), 1.9 M PhLi in dibutyl ether solution (8.3 mL, 15.7 mmol. 4 eq.) was added at 0° C. and the reaction mixture was stirred at room temperature for 30 minutes. The resulting mixture was cooled to −20° C. A solution of 2,4-O-benzylidene-D-threose (II-3) and its formate (II-4) (1 g, 3.93 mmol) in THF (7 mL) was added. The resulting mixture was stirred at the same temperature (−20° C.) for 2 hours and then at room temperature for another 1 hour. Methanol (7.5 mL) and water (12.5 mL) were then added to the reaction mixture and the mixture was vigorously stirred at room temperature for another 9 hours. The products were extracted with EtOAc, dried over Na₂SO₄, and the mixture was evaporated to dryness. The obtained crude was purified by a silica gel column chromatography using hexane:CH₂Cl₂=1:2 (by volume) as an eluant to obtain pure [2(R), 3(R), 4E]-1,3-O-benzylidene-4-icosene-2-ol (II-5a) (950 mg), pure [2(R), 3(R), 4Z]-1,3-O-benzylidene-4-icosene-2-ol (II-5c) (90 mg), and a mixture of II-5a and II-5c (121 mg). The overall reaction yield was 67%.

Example ii): To a solution of (1-hexadecyl) triphenylphosphonium bromide (39.0 g, 68.8 mmol, 1.25 eq.) and LiBr (19.0 g, 220.0 mmol, 4 eq.) in toluene (588 mL), 1.9 M PhLi in dibutyl ether solution (116 mL, 220.0 mmol, 4 eq.) was added at 0° C. and the reaction mixture was stirred at room temperature for 30 minutes. The resulting mixture was cooled to −20° C. A solution of 2,4-O-benzylidene-D-threose (II-3) and its formate (II-4) (14 g, 55.0 mmol) in THF (98 mL) was added. The resulting mixture was stirred at the same temperature (−20° C.) for 2.0 hours and then at room temperature for another 1 hour. Methanol (105 mL) and water (175 mL) were then added to the reaction mixture and the mixture was vigorously stirred at room temperature for another 9.0 hours. The products were extracted with EtOAc, dried over Na₂SO₄, and the mixture was evaporated to dryness. The obtained crude was purified by a silica gel column chromatography using hexane:EtOAc=4:1 (by volume) as an eluant to obtain 1,3-O-benzylidene-4-icosene-2-ol as a mixture of E (II-5a) and Z (II-5c) isomers (E:Z=10:1) (16.3 g) in 67% yield.

The obtained mixture of E and Z isomers was purified further by a silica gel column chromatography using hexane:CH₂Cl₂=1:2 (by volume) as an eluant to obtain pure [2(R), 3(R), 4E]-1,3-O-benzylidene-4-icosene-2-ol (II-5a) and pure [2(R), 3(R), 4Z]-1,3-O-benzylidene-4-icosene-2-ol (II-5c).

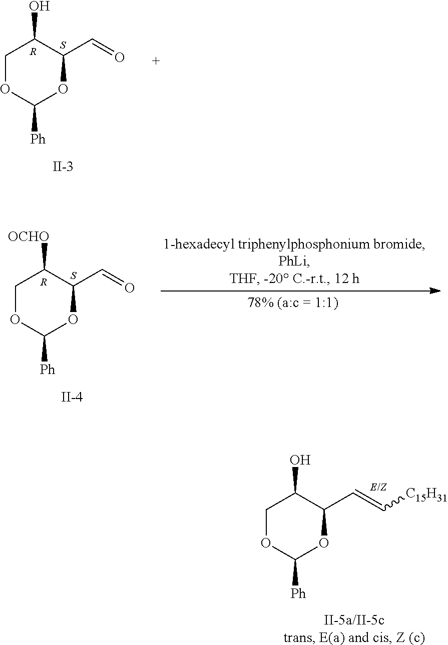

To a solution of (1-hexadecyl) triphenylphosphonium bromide (39.0 g, 68.8 mmol. 1.25 eq.) in THF (150 mL), 1.9 M PhLi in dibutyl ether solution (87 mL, 165.0 mmol, 3 eq.) was added at 0° C. and the reaction mixture was stirred at room temperature for 30 minutes. The resulting mixture was cooled to −20° C. A solution of 2,4-O-benzylidene-D-threose (II-3) and its formate (1-4) (14 g, 55.0 mmol) in THF (70 mL) was then added. The resulting mixture was stirred at the same temperature (−20° C.) for 2 hours and at room temperature for another 10 hours. Water was then added to the reaction mixture. The products were extracted with EtOAc, dried over Na₂SO₄, and evaporated to dryness. The obtained crude was purified by a silica gel column chromatography using hexane:EtOAc=4:1 (by volume) as an eluant to obtain 1,3-O-benzylidene-4-icosene-2-ol as a mixture of E (II-5a) and Z (II-5c) isomers (E:Z=1:1) (19.5 g) in 78% yield.

The obtained mixture of E and Z isomers was purified further by a silica gel column chromatography using hexane:CH₂Cl₂=2:3 (by volume) as an eluant to obtain pure [2(R), 3(R), 4E]-1,3-O-benzylidene-4-icosene-2-ol (II-5a) and pure [2(R), 3(R), 4Z]-1,3-O-benzylidene-4-icosene-2-ol (II-5c).

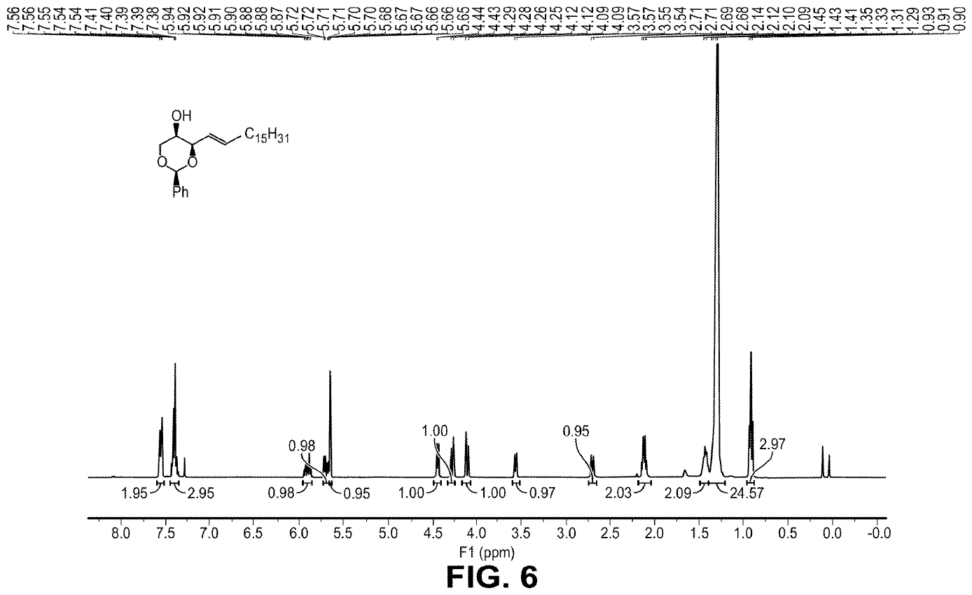

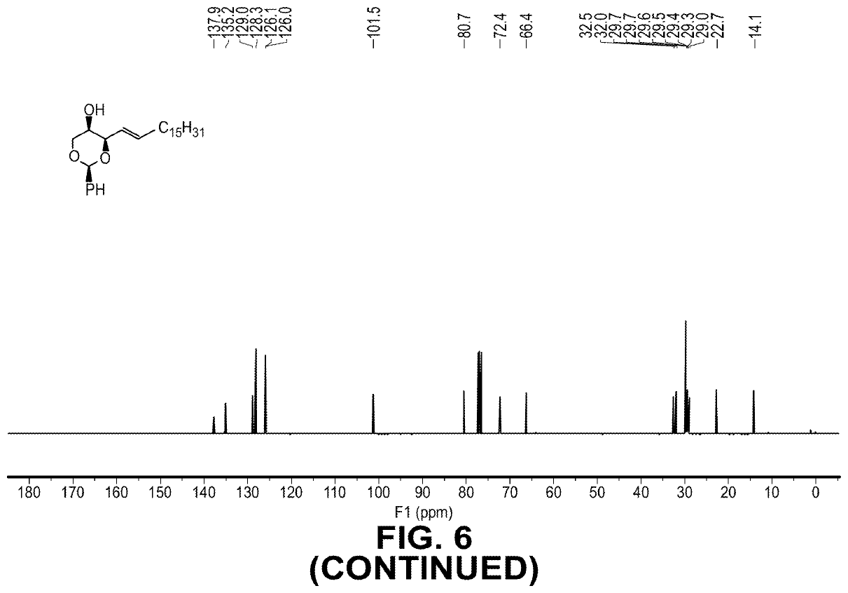

(II-5a) ¹H NMR (400 MHz, CDCl₃) δ 7.56-7.54 (m, 2H), 7.41-7.38 (m, 3H), 5.94-5.87 (m, 1H), 5.72-5.66 (m, 1H), 5.65 (s, 1H), 4.43 (d, J=4.0 Hz, 1H), 4.29-4.25 (dd, J=12.0, 3.0 Hz, 1H), 4.12-4.09 (dd, J=12.0, 3.0 Hz, 1H), 3.57-3.54 (dd, J=12, 3.0 Hz, 1H), 2.71-2.68 (d, J=12, 1H), 2.14-2.09 (m, 2H), 1.45-1.41 (m, 2H), 1.35-1.29 (m, 24H), 0.92 (t, J=6.0 Hz, 3H); ¹³C NMR (100 MHz, CDCl₃) δ 137.9, 135.2, 129.0, 128.3, 14.1, 126.1, 126.0, 101.5, 80.7, 72.4, 66.4, 32.5, 22.7, 31.9, 29.7, 29.69, 29.62, 29.5, 29.4, 29.3, 29.0. See FIG. 6 for ¹H and ¹³C NMR spectra of compound H₁-5a.

### 1,3-O-Benzylidene-octadec-4-ene-2-ol (II-5b and II-5d)

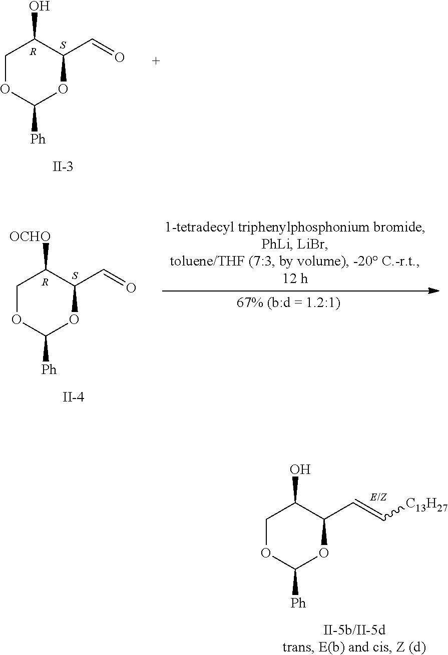

To a solution of (1-tetradecyl) triphenylphosphonium bromide (37.1 g, 68.8 mmol, 1.25 eq.) and LiBr (14.3 g, 165.0 mmol, 3 eq.) in 250 mL of toluene:THF=7:3 (by volume), 1.9 M PhLi in dibutyl ether solution (87 mL, 165.0 mmol, 3 eq.) was added at 0° C. and the reaction mixture was stirred at room temperature for 30 minutes. The resulting mixture was cooled to −20° C. A solution of 2,4-O-benzylidene-D-threose (II-3) and its formate (II-4) (14 g, 55.0 mmol) in THF (70 mL) was then added. The resulting mixture was stirred at the same temperature (−20° C.) for 2 hours and then at room temperature for another 11 hours. Water was then added to the reaction mixture. The products were extracted with EtOAc, dried over Na₂SO₄, and evaporated to dryness. The obtained crude was purified by a silica gel column chromatography using hexane:EtOAc=4:1 (by volume) as an eluant to obtain 1,3-O-benzylidene-octadec-4-ene-2-ol as a mixture of E (II-5b) and Z (II-5d) isomers (E:Z=1.2:1) (15.5 g) in 67% yield. The obtained mixture of E and Z was purified further by a silica gel column chromatography using hexane:CH₂Cl₂=1:2 (by volume) as an eluant to obtain pure [2(R), 3(R), 4E]-1,3-O-benzylidene-octadec-4-ene-2-ol (II-5b) and pure [2(R), 3(R), 4Z]-1,3-O-benzylidene-octadec-4-ene-2-ol (II-5d).

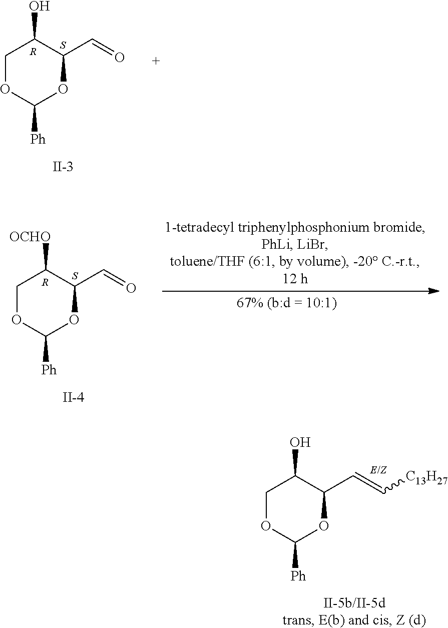

Example iii) To a solution of (1-tetradecyl) triphenylphosphonium bromide (2.65 g, 4.9 mmol, 1.25 eq.) and LiBr (1.36 g, 15.7 mmol, 4 eq.) in 42 mL of toluene. 1.9 M PhLi in dibutyl ether solution (8.3 mL, 15.7 mmol, 4 eq.) was added at 0° C. and the reaction mixture was stirred at room temperature for 30 minutes. The resulting mixture was cooled to −20° C. A solution of 2,4-O-benzylidene-D-threose (3) and its formate (4) (1 g, 3.93 mmol) in THF (7 mL) was then added. The resulting mixture was stirred at the same temperature (−20° C.) for 2 hours and then at room temperature for another 1 hour. Methanol (7.5 mL) and water (12.5 mL) were then added to the reaction mixture and the mixture was vigorously stirred at room temperature for 9 hours. The obtained crude was purified by a silica gel column chromatography using hexane:CH₂Cl₂=1:2 (by volume) as an eluant to obtain pure [2(R), 3(R), 4E]-1,3-O-benzylidene-4-octadec-4-ene-2-ol (II-5b) (910 mg), pure [2(R), 3(R), 4Z]-1,3-O-benzylidene-octadec-4-ene-2-ol (II-5d) (60 mg), and a mixture of II-5b and II-5d (95 mg). The overall reaction yield was 67%.

Example iv) To a solution of (1-tetradecyl) triphenylphosphonium bromide (37.1 g, 68.8 mmol, 1.25 eq.) and LiBr (19.0 g, 220.0 mmol, 4 eq.) in 588 mL of toluene, 1.9 M PhLi in dibutyl ether solution (116 mL, 220.0 mmol, 4 eq.) was added at 0° C. and the reaction mixture was stirred at room temperature for 30 minutes. The resulting mixture was cooled to −20° C. A solution of 2,4-O-benzylidene-D-threose (3) and its formate (4) (14 g. 55.0 mmol) in THF (98 mL) was then added. The resulting mixture was stirred at the same temperature (−20° C.) for 2 hours and then at room temperature for another 1 hour. Methanol (105 mL) and water (175 mL) were then added to the reaction mixture and the mixture was vigorously stirred at room temperature for 9.0 hours. The obtained crude was purified by a silica gel column chromatography using hexane:EtOAc=4:1 (by volume) as an eluant to obtain 1,3-O-benzylidene-octadec-4-ene-2-ol as a mixture of E (II-5b) and Z (II-5d) isomers (E:Z=10:1) (15.5 g) in 67% yield.

The obtained mixture of E and Z was purified further by a silica gel column chromatography using hexane:CH₂Cl₂=1:2 (by volume) as an eluant to obtain the pure [2(R), 3(R), 4E]-1,3-O-benzylidene-octadec-4-ene-2-ol (II-5b) and pure [2(R), 3(R), 4Z]-1,3-O-benzylidene-octadec-4-ene-2-ol (II-5d).

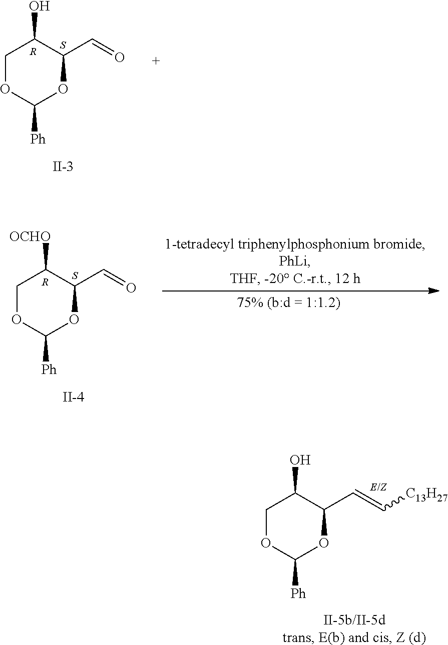

To a solution of (1-tetradecyl) triphenylphosphonium bromide (37.1 g, 68.8 mmol, 1.25 eq.) in THF, 1.9 M PhLi in dibutyl ether solution (87 mL, 165.0 mmol, 3 eq.) was added at 0° C. and the reaction mixture was stirred at room temperature for 30 minutes. The resulting mixture was cooled to −20° C. A solution of 2,4-O-benzylidene-D-threose (II-3) and its formate (II-4) (14 g, 55.0 mmol) in THF (70 mL) was added. The resulting mixture was stirred at the same temperature (−20° C.) for 2 hours and at room temperature for another 10 hours. Water was then added to the reaction mixture. The products were extracted with EtOAc, dried over Na₂SO₄, and evaporated to dryness. The obtained crude was purified by a silica gel column chromatography using hexane:EtOAc=4:1 (by volume) as an eluant to obtain 1,3-O-benzylidene-octadec-4-ene-2-ol as a mixture of E (II-5b) and Z (II-5d) isomers (E:Z=1:1.2) (17.3 g) in 75% yield. The mixture can be used for photo-isomerization in the next step.

The obtained mixture of E and Z was purified further by a silica gel column chromatography using hexane:CH₂Cl₂=2:3 (by volume) as an eluant to obtain pure [2(R), 3(R), 4E]-1,3-O-benzylidene-octadec-4-ene-2-ol (II-5b) and pure [2(R), 3(R), 4Z]-1,3-O-benzylidene-octadec-4-ene-2-ol (II-5d).

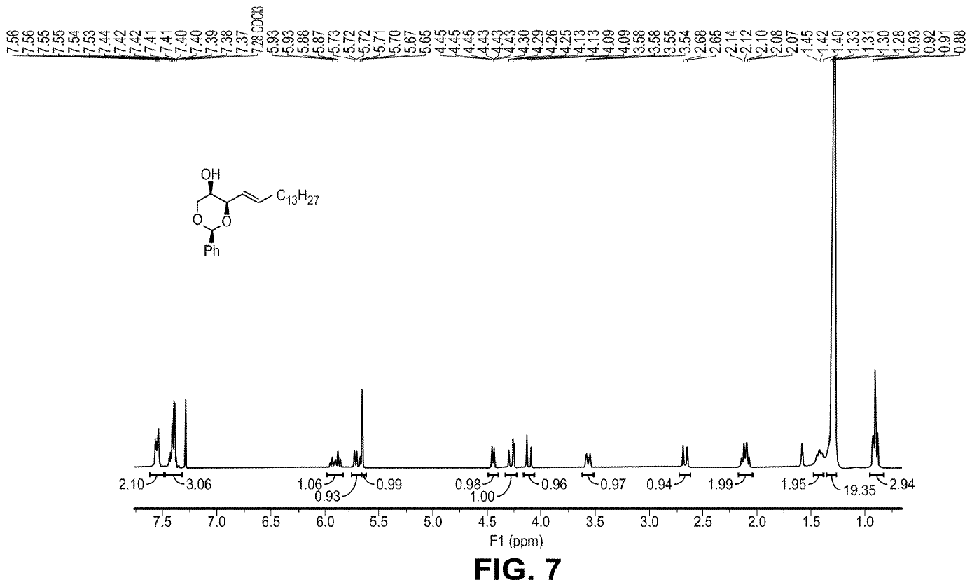

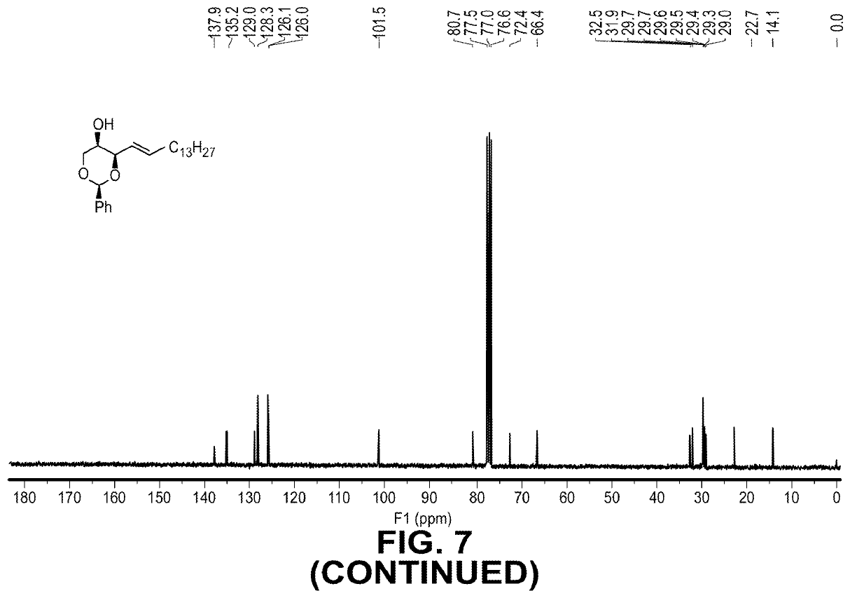

(II-5b)¹H NMR (300 MHz, CDCl₃) δ 7.52-7.56 (m, 2H), 7.35-7.45 (m, 3H), 5.95-5.85 (m, 1H), 5.73-5.66 (m, 1H), 5.65 (s, 1H), 4.45-4.43 (m, 1H), 4.30-4.25 (dd, J=12.0, 3.0 Hz, 1H), 4.13-4.09 (dd, J=12.0, 1.4 Hz, 1H), 2.65 (d, J=9.0 Hz, 1H), 2.14-2.07 (m, 2H), 1.45-1.40 (m, 2H), 1.33-1.28 (m, 20H), 0.91 (t, J=6 Hz, 3H); ¹³C NMR (75 MHz, CDCl₃) δ 137.9, 135.2, 129.0, 128.3, 126.1, 126.0, 101.5, 80.7, 72.4, 66.4, 32.5, 31.9, 29.7, 29.68, 29.61, 29.5, 29.4, 29.3, 29.0, 22.7, 14.1. See FIG. 7 for ¹H and ¹³C NMR spectra of compound II-5b.

(II-5d) ¹H NMR (300 MHz, Chloroform-d) δ 7.55-7.48 (dd, J=6.6, 3.2 Hz, 2H), 7.42-7.36 (dd, J=5.1, 2.1 Hz, 3H), 5.90-5.82 (m, 1H), 5.60-5.49 (m, 2H), 4.52-4.43 (m, 1H), 4.42-4.35 (dd, J=10.9, 5.0 Hz, 1H), 3.68 (t, J=10.9 Hz, 1H), 3.60-3.47 (ddd, J=10.7, 9.3, 5.0 Hz, 1H), 2.36-2.22 (ddd, J=9.2, 4.9, 1.8 Hz, 2H), 1.52-1.44 (m, 2H), 1.33-1.28 (m, 20H), 0.92 (t, J=6.0 Hz, 3H); ¹³C NMR (76 MHz, CDCl₃) 137.8, 129.1, 128.3, 126.2, 125.6, 101.1, 76.6, 69.2, 57.5, 32.0, 29.8, 29.7, 29.6, 29.5, 29.5, 29.4, 29.3, 28.3, 22.7, 14.2.

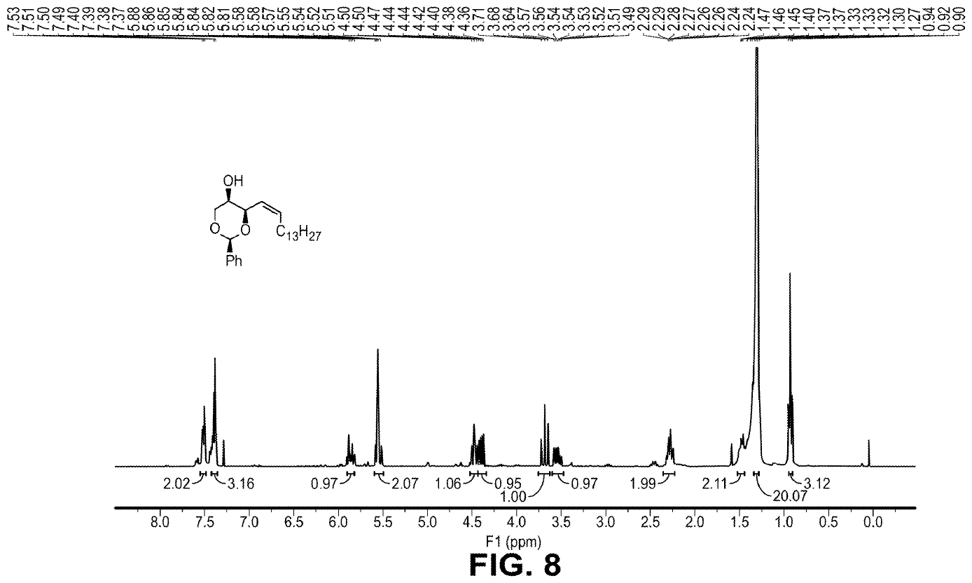

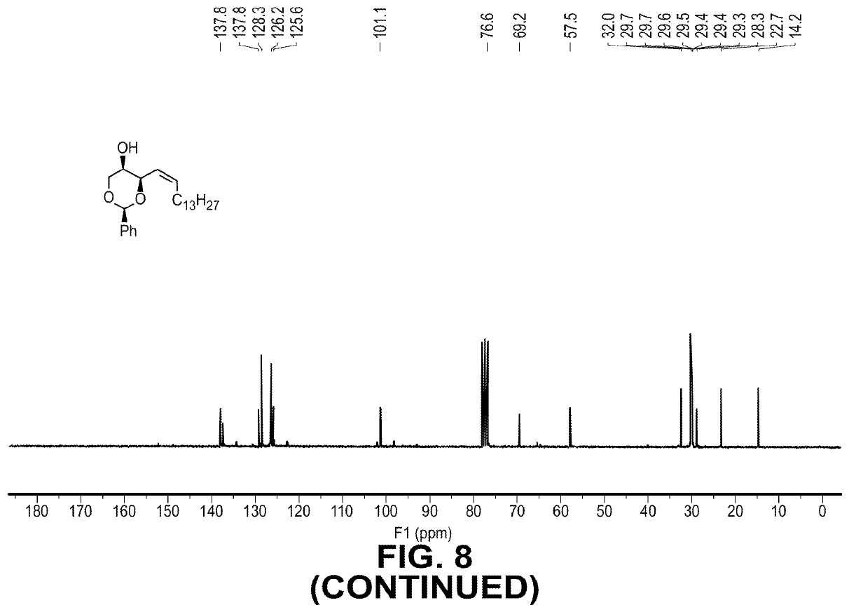

See FIG. 8 for ¹H and ¹³C NMR spectra of compound II-5d.

### Photo-isomerization and purification to obtain pure 1,3-O-benzylidene-4-icosene-2-ol (II-5a) and 1,3-O-benzylidene-octadec-4-ene-2-ol (II-5b)

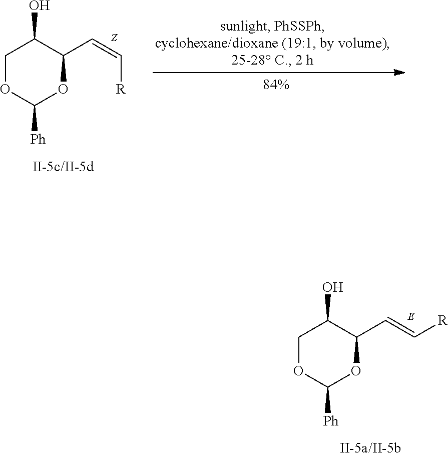

The Z-isomer II-5c or II-5d (1 g, 2.6 mmol) and diphenyl disulfide (0.62 g, 2.8 mmol, 1.1 equiv.) were added to a Pyrex round bottom flask and completely dissolved in cyclohexane: 1.4 dioxane=19:1 (by volume) (50 mL/1 g of II-5a-d). The solution in the round bottom flask was degassed, sealed, the air was exchanged with N₂ gas, and irradiated under the sunlight for 2 hours (25-28° C.). After the reaction reached equilibrium (2 hours) as monitored by thin-layer chromatography (TLC) using CH₂Cl₂:hexane=4:1 (by volume) as the developing solvent. The solvent was evaporated from the reaction mixture and the resulting residue was subjected to a silica gel column chromatography using CH₂Cl₂:hexane=3:2 (by volume) as an eluant to obtain pure E-isomer II-5a or II-5b (0.84 g) in 84% yield. The reaction was repeated with several times at 5 g and 10 g scales and similar yields (82-84%) were obtained.

### [2(S), 3(R), 4E]-2-Azido-4-icosene-1,3-diol (II-7a)

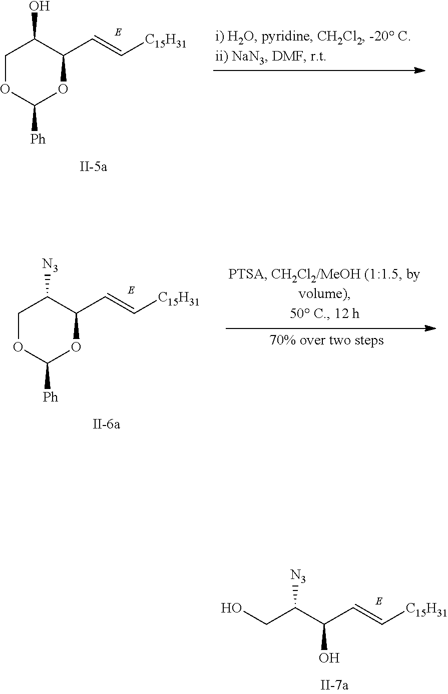

To a stirred solution of [2(R), 3(R), 4E]-1,3-O-benzylidene-4-icosene-2-ol (II-5a) (10 g, 24.0 mmol, 1 eq.) in CH₂Cl₂ (60 mL), pyridine (4.8 mL, 60.0 mmol, 2.5 eq.) was added, and the reaction mixture was stirred at −20° C. Trifluoromethanesulfonic anhydride (4.8 mL, 28.8 mmol, 1.2 eq.) was added to the stirred solution, and the stirring was continued at the same temperature (−20° C.). After the reaction was completed (1 hour) as monitored by TLC using hexane:ethyl acetate=5:1 (by volume) as the developing solvent, N,N-dimethylformamide (DMF) (120 mL) and sodium azide (6.24 g, 96.0 mmol, 4 eq.) were added in situ, and the mixture was stirred at room temperature overnight. The solvent was then evaporated, and the residue was extracted with CH₂Cl₂. The extract was washed with water, dried, and the solvent was evaporated, to obtain the crude [2(S), 3(R), 4E]-2-azido-1,3-O-benzylidene-4-icosene (II-6a) (11 g) as a syrup.

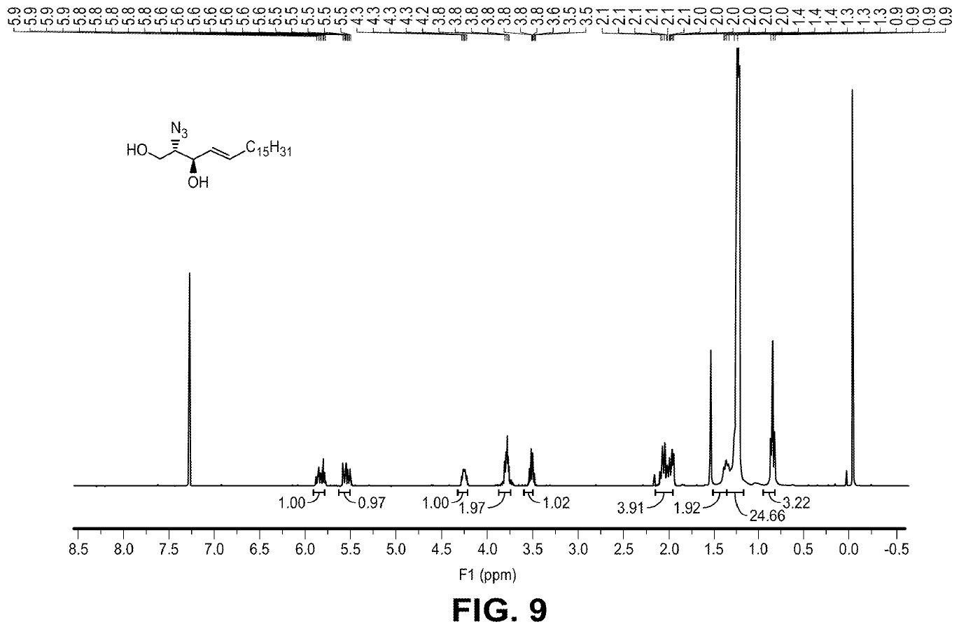

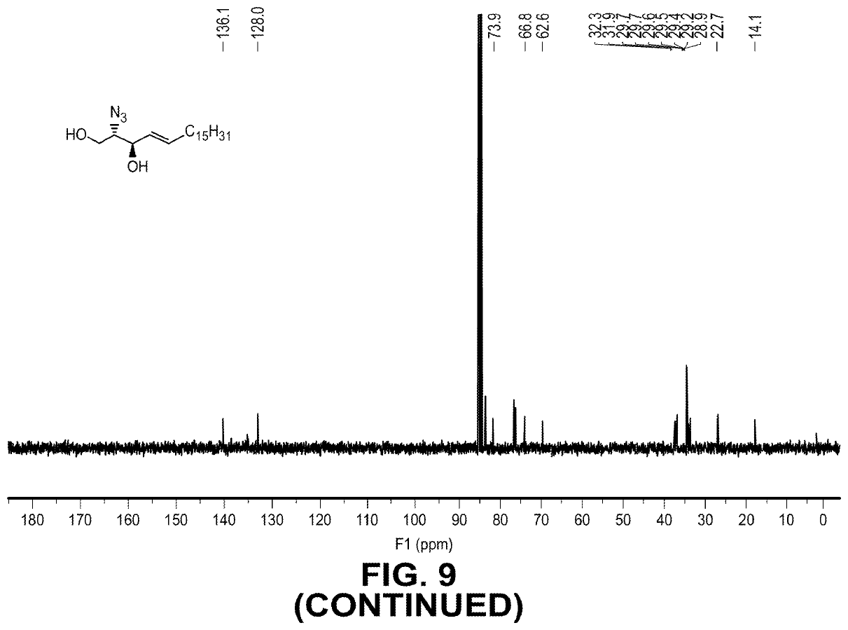

To a stirred solution of crude II-6a (11 g, 21.7 mmol. 1.0 eq.) in 100 mL of 1:1.5 mixture of CH₂Cl₂ and MeOH, p-toluenesulfonic acid (3.75 g, 21.7 mmol, 1.0 eq.) was added, and the mixture was stirred at 50° C. for 12 hours, neutralized by adding Et₃N, and evaporated to dryness. The obtained crude was purified by a silica gel column chromatography using hexane:EtOAc=2:1 (by volume) as an eluant to obtain [2(S), 3(R), 4E]-2-azido-4-icosene-1,3-diol (II-7a) (5.9 g) as a white amorphous solid in 70% yield (over two steps from II-5a). ¹H NMR (300 MHz, CDCl₃) δ 5.90-5.80 (ddd, J=15.7, 7.2, 6.2 Hz, 1H), 5.61-5.52 (ddt, J=15.4, 7.3, 1.4 Hz, 1H), 3.83-3.79 (m, 2H), 4.30-4.25 (m, 1H), 3.56-3.51 (m, 1H), 2.13-1.99 (m, 4H), 1.44-1.39 (m, 2H), 1.33-1.28 (m, 24H), 0.90 (t, 6.0 Hz, 3H); ¹³C NMR (75 MHz, CDCl₃) δ 136.1, 128.0, 73.9, 66.8, 62.6, 32.3, 31.9, 29.8, 29.7, 29.6, 29.5, 29.4, 29.2, 28.9, 22.7, 14.1. See FIG. 9 for ¹H and ¹³C NMR spectra of compound II-7a. [2(S), 3(R), 4E]-2-Azido-octadec-4-ene-1,3-diol (II-7b)

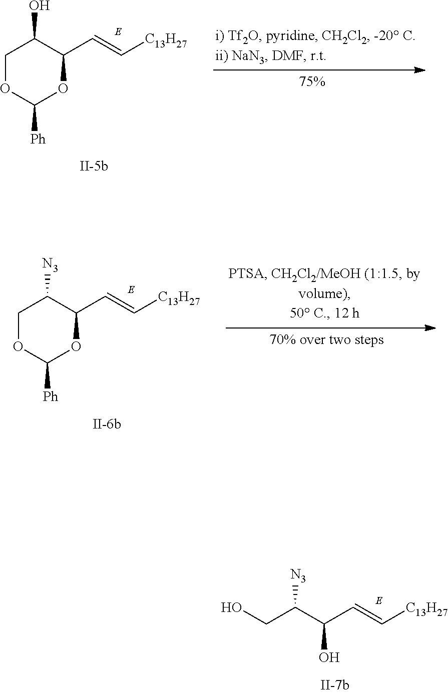

To a stirred solution of [2(R), 3(R), 4E]-1,3-O-benzylidene-octadec-4-ene-2-ol (II-5b) (10 g, 25.7 mmol, 1.0 eq.) in CH₂Cl₂ (60 mL), pyridine (5.2 mL, 64.2 mmol, 2.5 eq.) was added and the reaction mixture was stirred at −20° C. Trifluoromethanesulfonic anhydride (4.8 mL, 28.6 mmol, 1.2 eq.) was added To the stirred solution, and the stirring was continued at same temperature (−20° C.). After the reaction was completed (1 hour) as monitored by TLC using hexane:ethyl acetate=5:1 (by volume) as a developing solvent, DMF (120 mL) and sodium azide (6.7 g, 102.8 mmol, 4.0 eq.) were added in situ, and the mixture was stirred at room temperature overnight. The solvent was then evaporated, and the residue was extracted with CH₂Cl₂. The extract was washed with water, dried, and the solvent was evaporated, to obtain the crude [2(s), 3(R), 4E]-2-azido-1,3-O-benzylidene-octadec-4-ene (II-6b) (11 g) as a syrup.

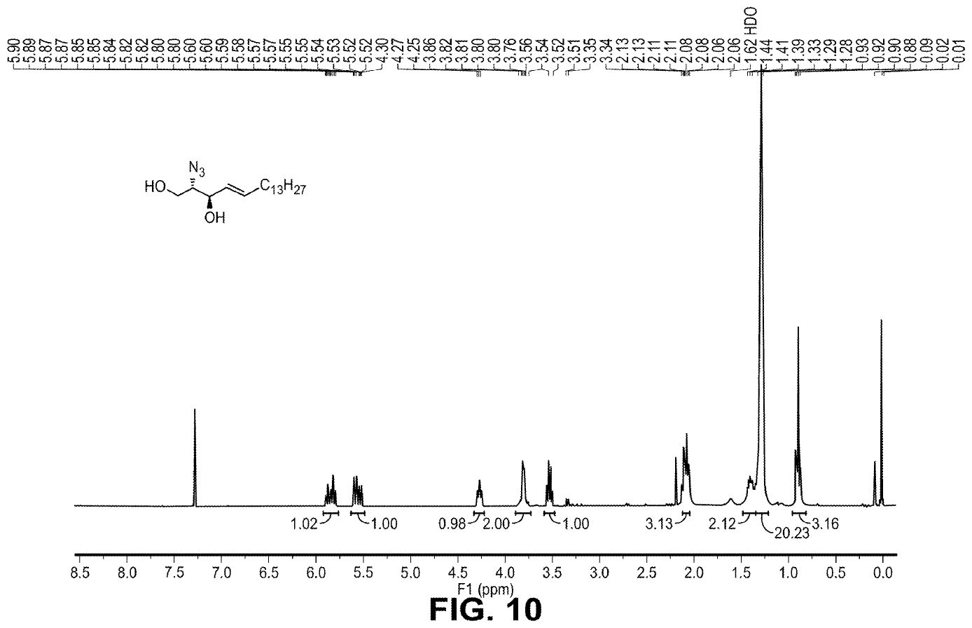

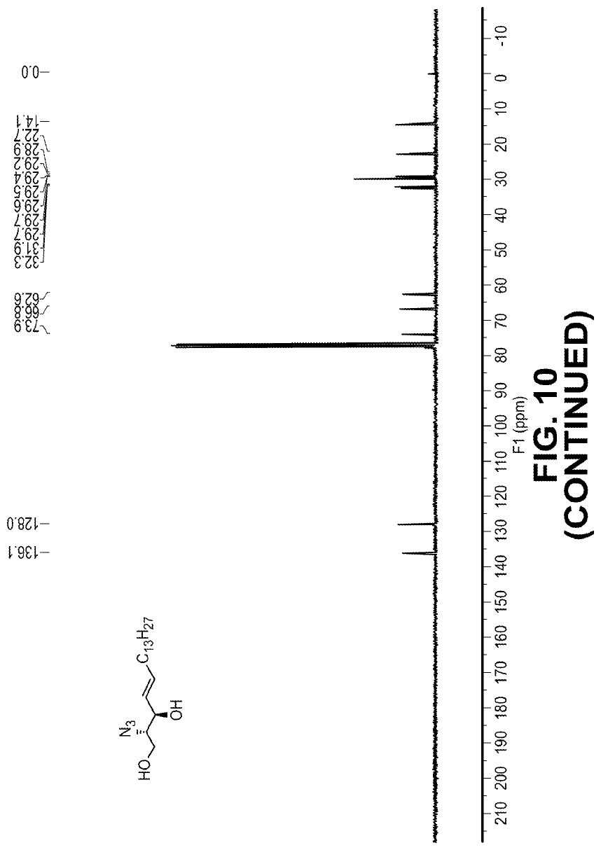

To a stirred solution of crude II-6b (11 g, 23.0 mmol, 1 eq.) in 100 mL of 1:1.5 mixture of CH₂Cl₂ and MeOH, p-toluenesulfonic acid (3.96 g, 23.0 mmol, 1.0 eq.) was added and the mixture was stirred at 50° C. temperature for 12 hours, neutralized by adding Et₃N, and evaporated to dryness. The obtained crude was purified by a silica gel column chromatography using hexane:EtOAc=2:1 (by volume) as an eluant to obtain II-7b (5.9 g) as a white amorphous solid in 70% yield (over two steps from II-5b). ¹H NMR (300 MHz, CDCl₃) δ 5.90-5.80 (dtd, J=14.5, 6.7, 1.0 Hz, 1H), 5.60-5.52 (ddt, J=15.4, 7.3, 1.4 Hz, 1H), 4.27 (t, J=6.0 Hz, 1H), 3.86-3.76 (m, 2H), 3.56-3.51 (m, 1H), 2.13-2.06 (m, 3H), 1.44-1.39 (m, 2H), 1.33-1.28 (m, 20H), 0.90 (m, 3H); ¹³C NMR (75 MHz, CDCl₃) δ 136.1, 128.0, 73.9, 66.8, 62.6, 32.3, 31.9, 29.8, 29.7, 29.6, 29.5, 29.4, 29.2, 28.9, 22.7, 14.1. See FIG. 10 for ¹H and ¹³C NMR spectra of compound II-7b.

### Synthesis of 2-azido-3-benzoyl sphingosine (d20:1) (II-8a) from 2-azido-sphingosine (d20:1) (1-7a)

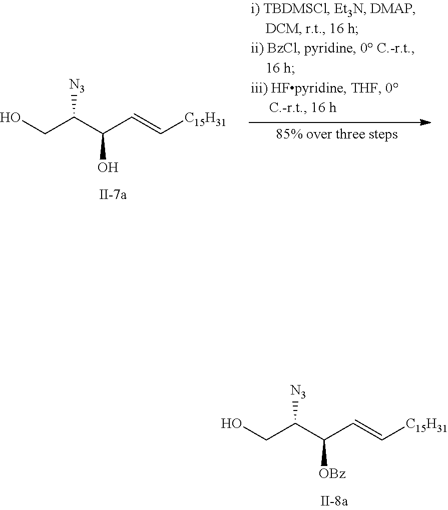

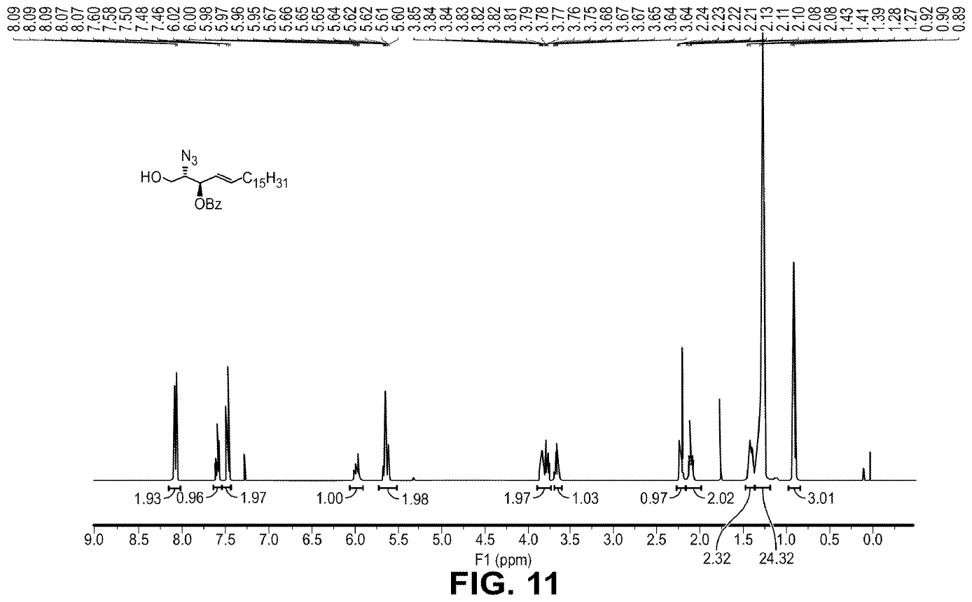

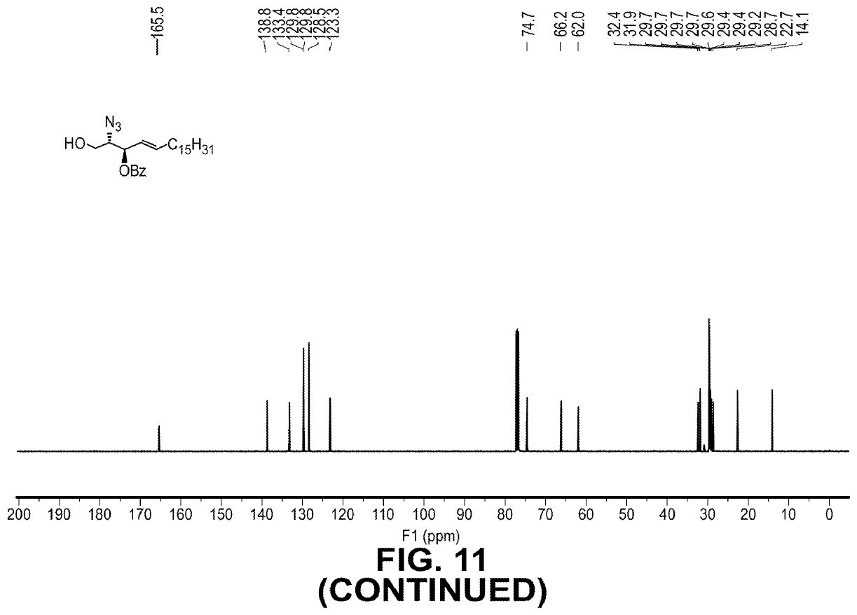

To a stirred solution of [2(S), 3(R), 4E]-2-azido-icos-4-ene-1,3-diol (II-7a) (5 g, 14.2 mmol, 1 eq.) in 50 mL of 4:1 mixture of CH₂Cl₂ and DMF, Et₃N (7 mL, 49.7 mmol, 3.5 eq.), DMAP (87.0 mg, 0.71 mmol, 0.05 eq.), and TBDPSCl (4.4 mL, 17.0 mmol, 1.2 eq.) were added. The mixture was stirred at room temperature (r.t.) for 12 hours (h) and then Et₃N (7 mL, 3.5 eq.) and benzoyl chloride (2.0 mL, 17.0 mmo, 1.2 eq.) were added. The reaction mixture was stirred at r.t. for another 12 hours, diluted with EtOAc, washed with 1 N HCl solution followed by saturated NaHCO₃ solution. The organic layer was separated, dried over Na₂SO₄, and evaporated to dryness. The obtained crude was dissolved in THF, transferred to a plastic flask and treated with 5.5 mL of HF·pyridine solution. The reaction was continued for 12 hours at r.t. The reaction mixture was quenched by adding solid NaHCO₃ pinch by pinch until the pH of the mixture became basic. The mixture was diluted with EtOAc, washed with H₂O, and the organic layer was separated, dried over Na₂SO₄, and evaporated to dryness. The obtained crude was purified by a silica gel column chromatography using hexane:EtOAc=6:1 (by volume) as an eluant to obtain pure [2(S), 3(R), 4E]-2-azido-3-O-benzoyl-icos-4-ene-1-ol (II-8a) (5.5 g) as a white amorphous solid in 85% yield. ¹H NMR (400 MHz, CDCl₃) δ=8.14-8.02 (m, 2H), 7.64-7.55 (d, J=7.4, 1H), 7.53-7.41 (t, J=7.7, 2H), 6.05-5.91 (dt, J=14.0, 6.9, 1H), 5.73-5.55 (m, 2H), 3.88-3.73 (m, 2H), 3.71-3.59 (ddd, J=11.8, 7.1, 4.8, 1H), 2.25-2.20 (dd, J=7.8, 4.9, 1H), 2.16-2.03 (q, J=6.8, 2H), 1.46-1.35 (t, J=7.2, 2H), 1.36-1.20 (d, J=5.0, 24H), 0.96-0.82 (t, J=6.8, 3H); ¹³C NMR (100 MHz, CDCl₃) δ 165.5, 138.8, 133.4, 129.8, 129.7, 128.5, 123.3, 74.7, 66.2, 62.0, 32.4, 31.9, 29.8, 29.78, 29.75, 29.7, 29.6, 29.45, 29.4, 29.2, 28.7, 22.7, 14.1. See FIG. 11 for ¹H and ¹³C NMR spectra of compound II-8a.

### Synthesis of 2-azido 3-benzoyl sphingosine (d18:1) (II-8b) from 2-azido sphingosine (d18:1) (II-7b)

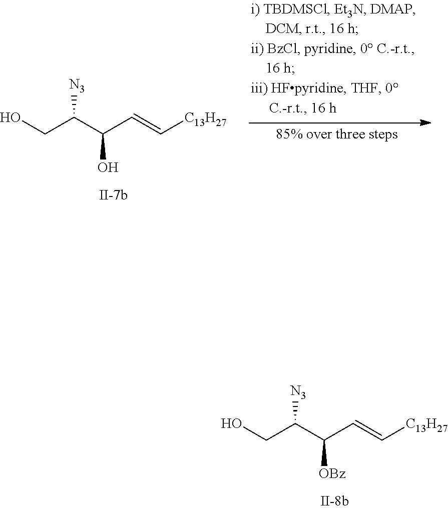

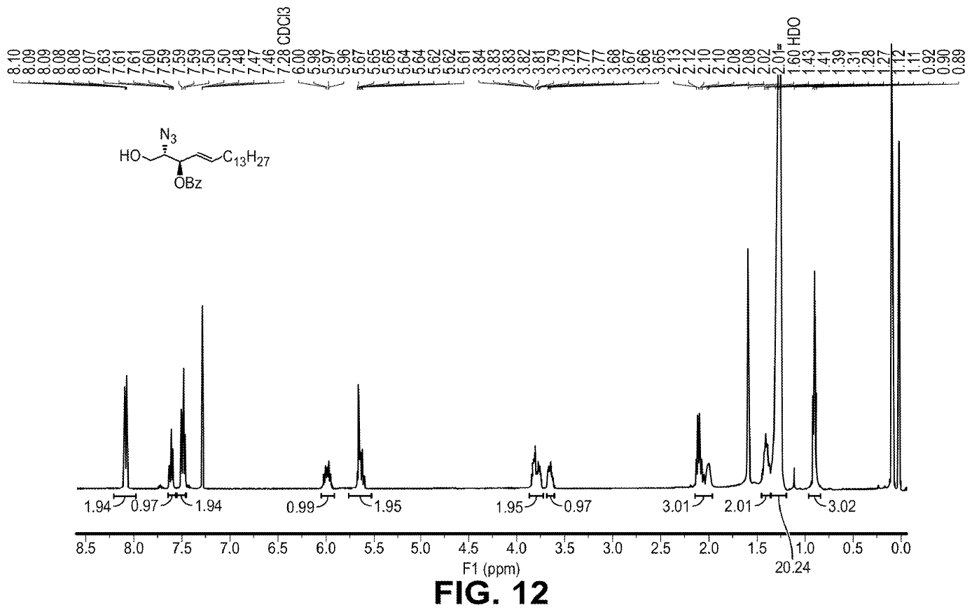

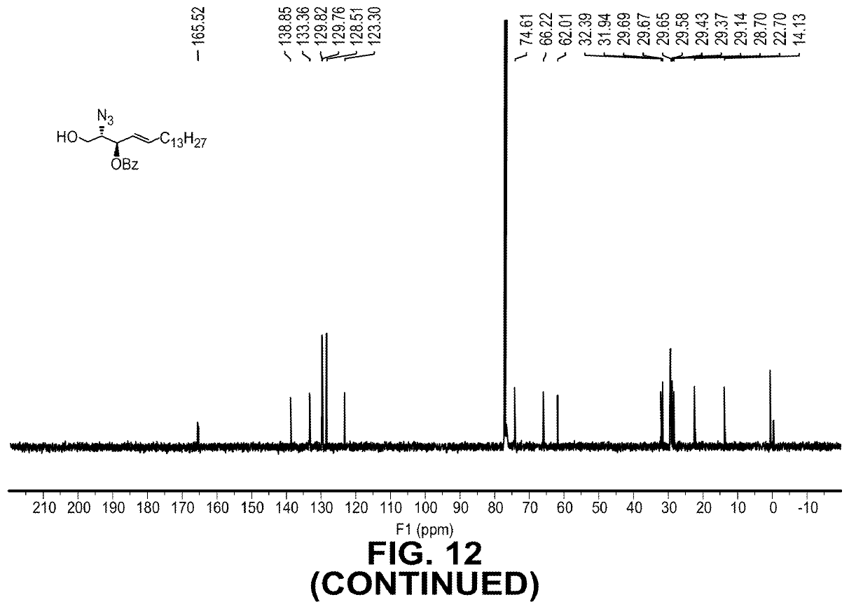

To a stirred solution of [2(S), 3(R), 4E]-2-azido-octadec-4-ene-1,3-diol (II-7b) (5 g, 15.3 mmol, 1 eq.) in 50 mL of 4:1 mixture of CH₂Cl₂ and DMF, Et₃N (6.4 mL, 53.5 mmol, 3.5 eq.), DMAP (93.0 mg, 0.77 mmol. 0.05 eq.), and TBDPSCl (5 mL, 18.3 mmol. 1.2 eq.) were added. The mixture was stirred at room temperature for 12 hours and then Et₃N (6.4 mL, 3.5 eq.) and benzoyl-chloride (2.1 mL, 18.3 mmol, 1.2 eq.) were added. The reaction mixture was stirred at room temperature for another 12 hours, diluted with EtOAc, washed with 1 N HCl solution followed by saturated NaHCO₃ solution. The organic layer was separated, dried over Na₂SO₄, and evaporated to dryness. The obtained crude was dissolved in THF, transferred to a plastic flask and treated with 5.5 mL of HF·pyridine solution. The reaction was continued for 12 hours at room temperature. The reaction mixture was quenched by adding solid NaHCO₃ pinch by pinch until the pH of the mixture became basic. The mixture was diluted with EtOAc, washed with H₂O, and the organic layer was separated, dried over Na₂SO₄, and evaporated to dryness. The obtained crude was purified by a silica gel column chromatography using hexane:EtOAc=6:1 (by volume) as an eluant to obtain pure [2(S), 3(R), 4E]-2-azido-3-O-benzoyl-octadec-4-ene-1-ol (II-8) (5.6 g) as a white amorphous solid in 85% yield. ¹H NMR (400 MHz, CDCl₃) δ 8.10-8.07 (m, 2H), 7.63-7.59 (m, 1H), 7.50-7.46 (m, 2H), 6.02-5.94 (m, 1H), 5.67-5.60 (m, 2H), 3.85-3.76 (m, 2H), 3.68-3.63 (m, 1H), 2.13-2.01 (m, 3H), 1.43-1.39 (m, 2H), 1.31-1.27 (m, 20H), 0.90 (t, J=6.0 Hz, 3H); ¹³C NMR (100 MHz, CDCl₃) δ 165.5, 138.8, 133.4, 129.8, 129.7, 128.5, 123.3, 74.6, 66.2, 62.0, 32.4, 31.9, 29.69, 29.67, 29.65, 29.6, 29.4, 29.3, 29.1, 28.7, 22.7, 14.1. See FIG. 12 for ¹H and ¹³C NMR spectra of compound II-8b.

### Chemical synthesis of lactosylsphingosines LacPSph (d20:1) (II-11a) and LacPSph (d18:1) (II-11b)

### 1,2,3,6-Tetra-O-benzoyl-4-O-(2,3,4,6-tetra-O-benzoyl-β-D-galactopyranosyl)-α/β-D-glucopyranosyl trichloroacetimidate (II-9)

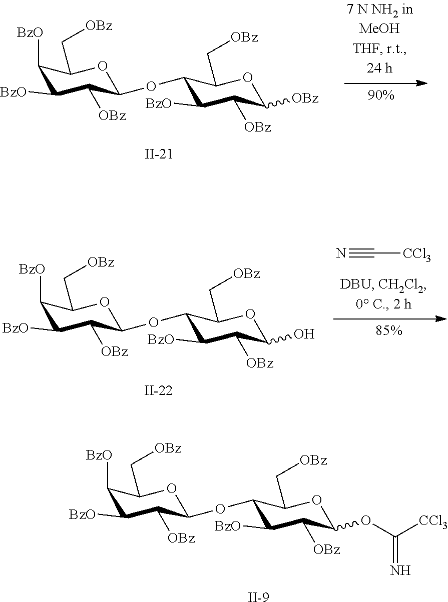

To a solution of per-O-benzoylated lactose (II-21) (10 g, 8.5 mmol) in dry THF (40 mL). 7 N NH₃ in MeOH (20 mL) was added and the mixture was stirred at room temperature under an inert atmosphere (N₂) for 12 hours before the mixture was diluted with EtOAc (100 mL). The organic layer was washed sequentially with HCl (1 N) and a saturated aqueous solution of NaHCO₃, and dried over Na₂SO₄. The solvents were removed under a reduced pressure. The residue was dried in vacuo and the obtained crude was purified by a silica gel column chromatography using hexane:EtOAc=2:1 (by volume) as an eluant to obtain pure hemiacetal 11-22 (8.2 g) in 90% yield.

To a solution of per-O-benzoylated lactose hemiacetal (II-22) (8 g, 7.4 mmol, 1 eq.) in dry CH₂Cl₂ (100 mL), trichloroacetonitrile (4.5 mL, 44.8 mmol, 6 eq.) was added and the reaction mixture was stirred under an inert atmosphere at 0° C. for 15 min. 1,8-Diazabicyclo[5.4.0]undec-7-ene (DBU) (670 μL, 4.5 mmol, 0.6 eq.) was added to the stirred reaction mixture and the mixture was stirred it at 0° C. for 2 hours. The solvents were then removed under a reduced pressure and the product was purified by a silica gel column chromatography using hexane:EtOAc=1.5:1 (by volume) as an eluent to produce benzoylated lactose trichloroacetimadate derivative (II-9) (7.8 g) as an amorphous solid in 85% yield. The residue was dried in vacuo and used in the next step without further characterization.

### O-(2,3,4,6-Tetra-O-benzoyl-β-D-galactopyranosyl)-(1→4)-(2,3,6-tri-O-benzoyl-β-D-glucopyranosyl)-(1→1)-(2S, 3R, 4E)-2-azido-icos-4-ene-3-ol (II-10a)

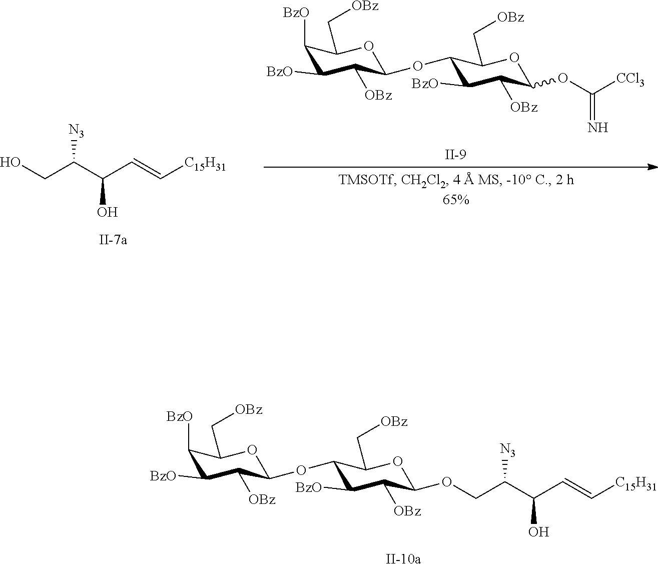

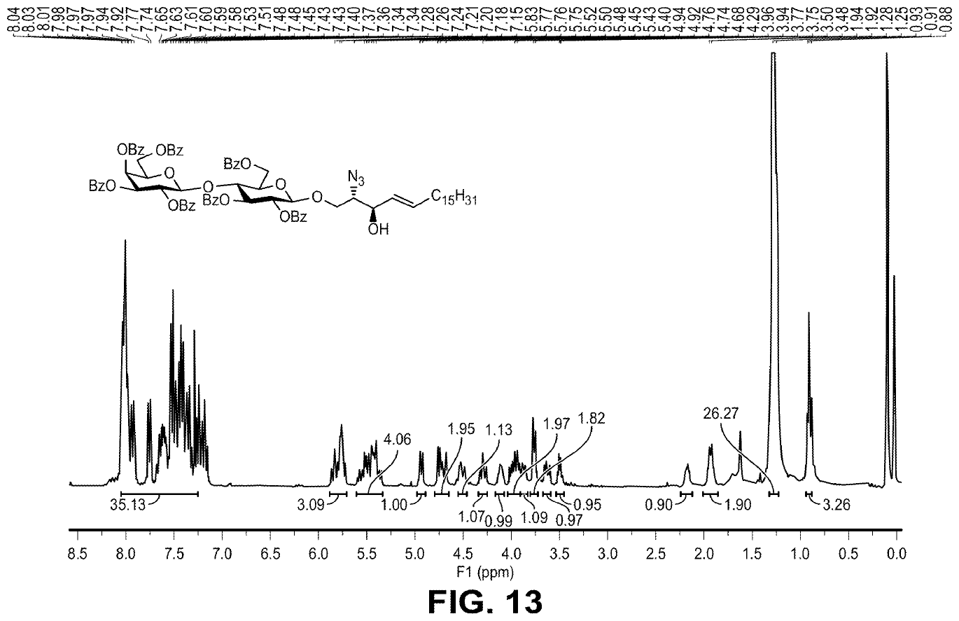

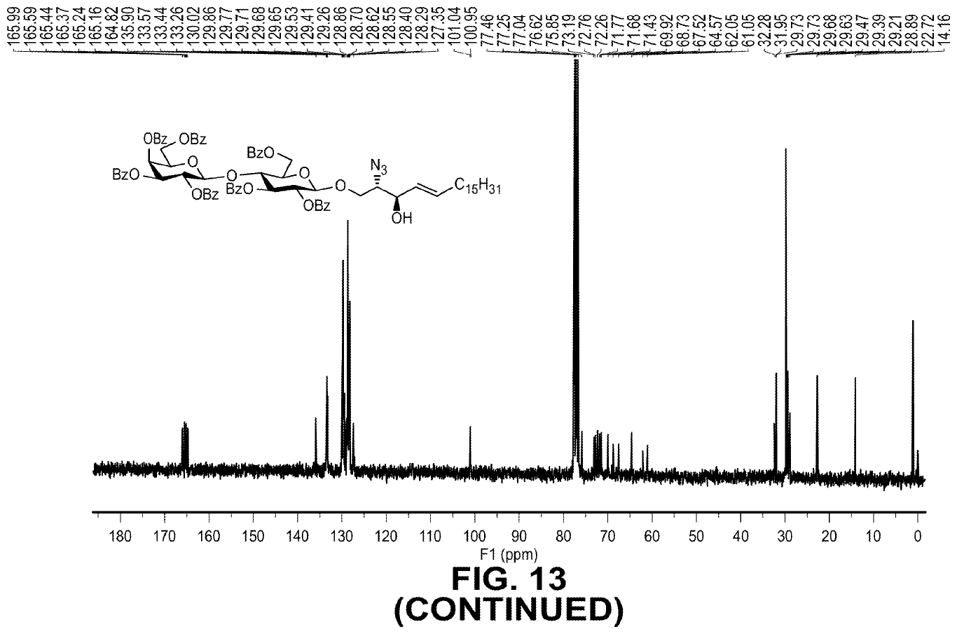

To a solution of (2R, 3S, 4E)-2-azido-4-icosene-1,3-diol (II-7a) (2 g, 5.65 mmol, 1 eq.) and per-O-benzoylated lactosyl trichloroacetimidate (II-9) (10.3 g, 8.5 mmol, 1.5 eq.) in 60 mL of dry CH₂Cl₂, powdered molecular sieves (4 Å) was added. The mixture was stirred under argon at room temperature for 30 minutes (min.). The reaction mixture was cooled down to −10° C. and TMSOTf (160 μL, 0.1 eq.) was added. The reaction mixture was then stirred at −10° C. for 2 hours. The reaction was quenched with Et₃N, and the solid was filtered off. The filtrate was concentrated under vacuum, and the residue was purified by a silica gel column chromatography using toluene:EtOAc=10:1 (by volume) as an eluent to produce compound II-10a (5.1 g) as an amorphous solid in 65% yield. ¹H NMR (300 MHz, CDCl₃) δ 8.04-7.15 (m, 35H), 5.86-5.72 (m, 3H), 5.57-5.35 (m, 4H), 4.94-4.92 (d, J=7.8 Hz, 1H), 4.76-4.68 (m, 2H), 4.53-4.48 (m, 1H), 4.29 (t, J=9.0 Hz, 1H), 4.12-4.02 (m, 1H), 3.98-3.88 (m, 2H), 3.85-3.84 (m, 1H), 3.77-3.75 (m, 2H), 3.65-3.60 (dd, J=10.5, 4.9 Hz, 1H), 3.52-3.48 (m, 1H), 2.17-2.15 (m, 1H), 1.96-1.90 (m, 2H), 1.28-1.25 (m, 26H), 0.91 (t, J=6.0 Hz, 3H); ¹³C NMR (75 MHz, CDCl₃) δ 166.0, 165.6, 165.5, 165.4, 165.2, 165.1, 164.8, 135.9, 133.6, 133.4, 133.3, 130.0, 129.9, 129.8, 129.71, 129.68, 129.65, 129.5, 129.4, 129.3, 128.9, 128.7, 128.6, 128.5, 128.4, 128.3, 127.4, 101.0, 100.9, 75.9, 73.2, 72.8, 72.3, 71.8, 71.7, 71.4, 69.9, 68.7, 67.5, 64.6, 62.0, 61.1, 32.3, 31.9, 29.73, 29.71, 29.68, 29.6, 29.5, 29.4, 29.2, 28.9, 22.7, 14.2. See FIG. 13 for ¹H and ¹³C NMR spectra of compound II-10a.

### O-(β-D-Galactopyranosyl)-(1→4)-(β-D-glucopyranosyl)-(1→1)-(2S, 3R, 4E)-2-amino-icos-4-ene LacβSph (d20:1) (II-11a)

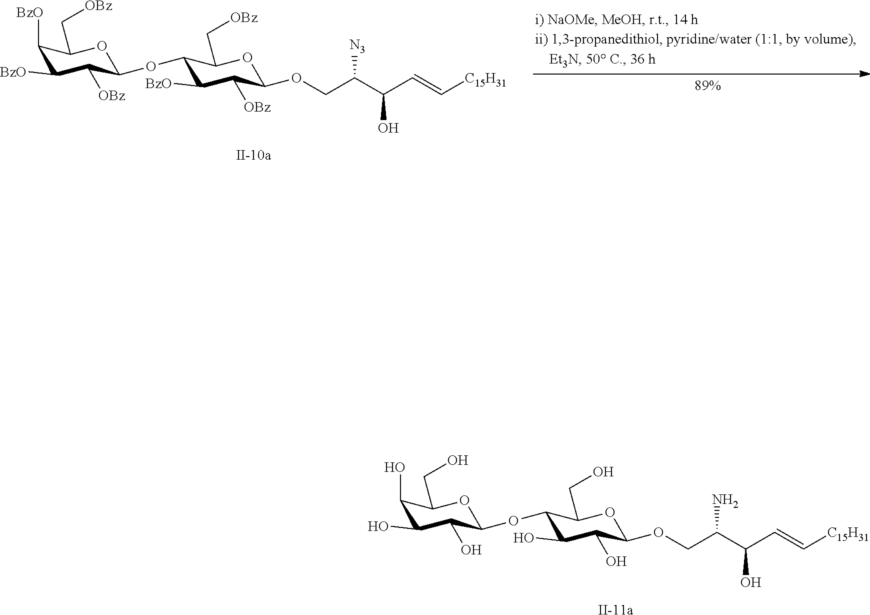

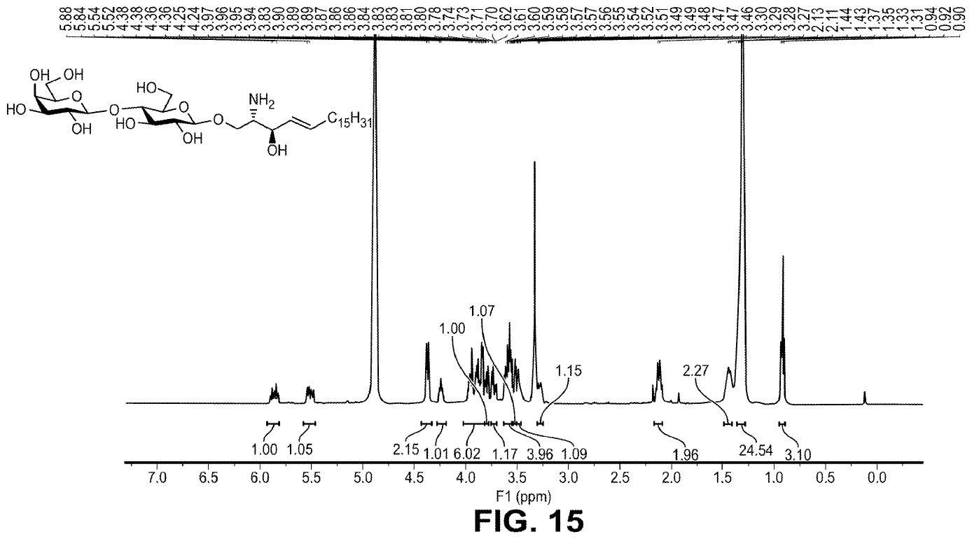

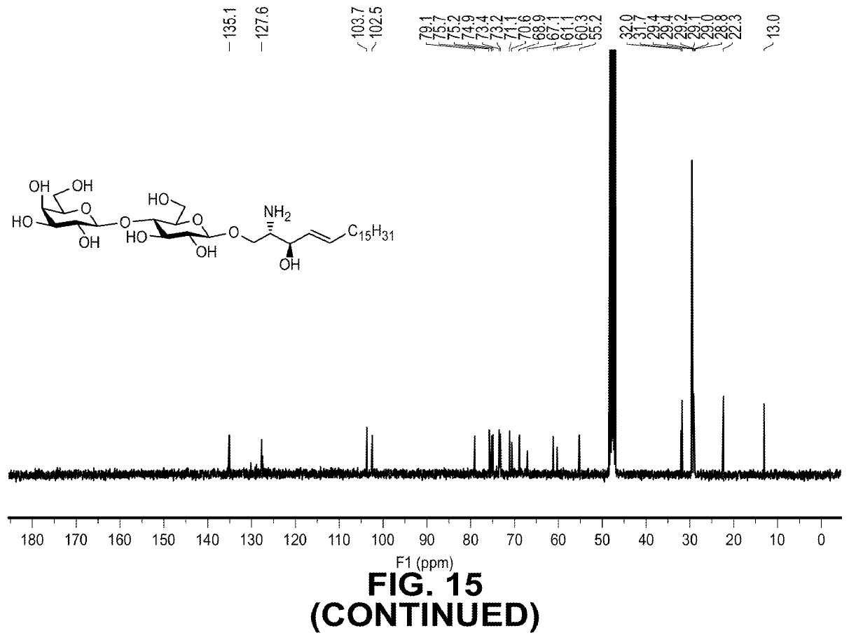

To a solution of II-10a (5 g, 3.5 mmol) in dry MeOH (150 mL), 30% NaOMe in MeOH (6 mL) was added. After being stirred at room temperature for 14 hours, the reaction mixture was neutralized with Dowex® 50W (H⁺), filtered, and concentrated under a reduced pressure. This intermediate was used in the next step without further purification. To the dry intermediate in pyridine:water=1:1 (by volume). 1,3-propanedithiol (3.6 mL, 35.0 mmol, 10 eq.) and Et₃N (5 mL) were added and the mixture was stirred at 50° C. for 36 hours. The reaction mixture was concentrated and purified by a silica gel column chromatography using chloroform:methanol:water=5:4:1 (by volume) as an eluent to produce LacPSph (d20:1) (II-11a) (2 g) as a white amorphous powder in 89% yield. ¹H NMR (400 MHz, CD₃OD) δ 5.89-5.82 (dt, J=14.3, 6.8 Hz, 1H), 5.54-5.48 (dd, J=15.4, 6.9 Hz, 1H), 4.38-4.36 (dd, J=7.7, 2.0 Hz, 2H), 4.24 (t, J=9.0 Hz, 1H), 3.98-3.83 (m, 6H), 3.83-3.78 (m, 1H), 3.74-3.70 (dd, J=12.0, 4.0 Hz, 1H), 3.62-3.54 (m, 4H), 3.52-3.51 (d, J=4.0 Hz, 1H), 3.49-3.46 (m, 1H), 3.26-3.28 (m, 1H), 2.13-2.11 (m, 2H), 1.44-1.43 (m, 2H), 1.37-1.31 (m, 24H), 0.92 (t, J=6.0 Hz, 3H); ¹³C NMR (100 MHz, CD₃OD) δ 135.1, 127.6, 103.7, 102.5, 79.1, 75.7, 75.2, 74.9, 73.4, 73.2, 71.1, 70.6, 13.0, 68.9, 67.1, 61.1, 60.3, 55.2, 32.0, 31.7, 29.5, 29.4, 29.2, 29.1, 29.0, 28.8, 22.3, 13.0. See FIG. 15 for ¹H and ¹³C NMR spectra of compound II-11a.

### O-(2,3,4,6-Tetra-O-benzoyl-β-D-galactopyranosyl)-(1→4)-(2,3,6-tri-O-benzoyl-β-D-glucopyranosyl)-(1→1)-(2S, 3R, 4E)-2-azido-octadec-4-ene-3-ol (II-10b)

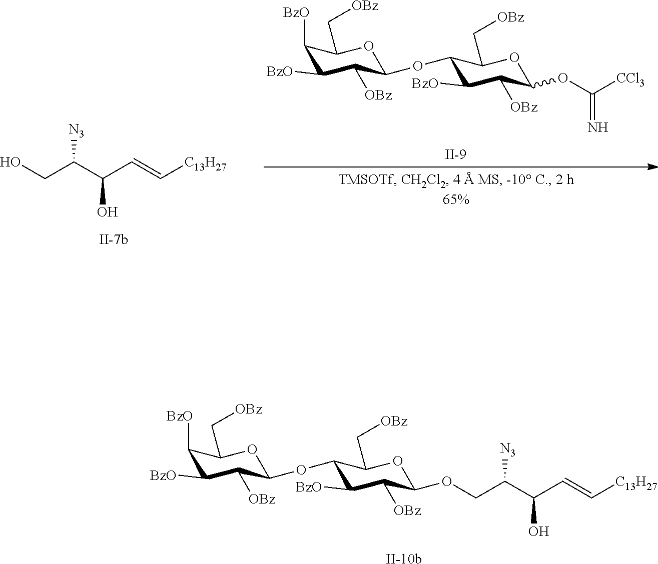

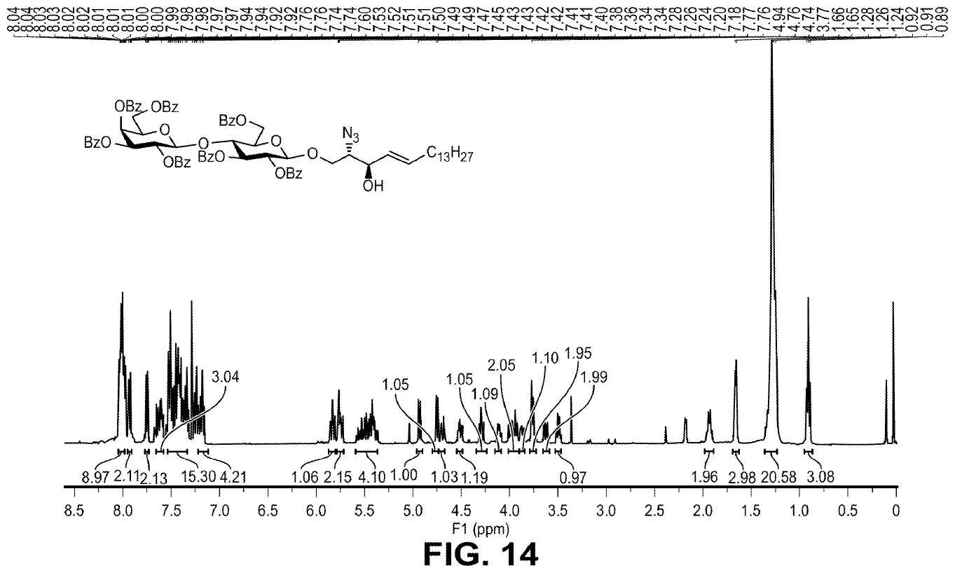

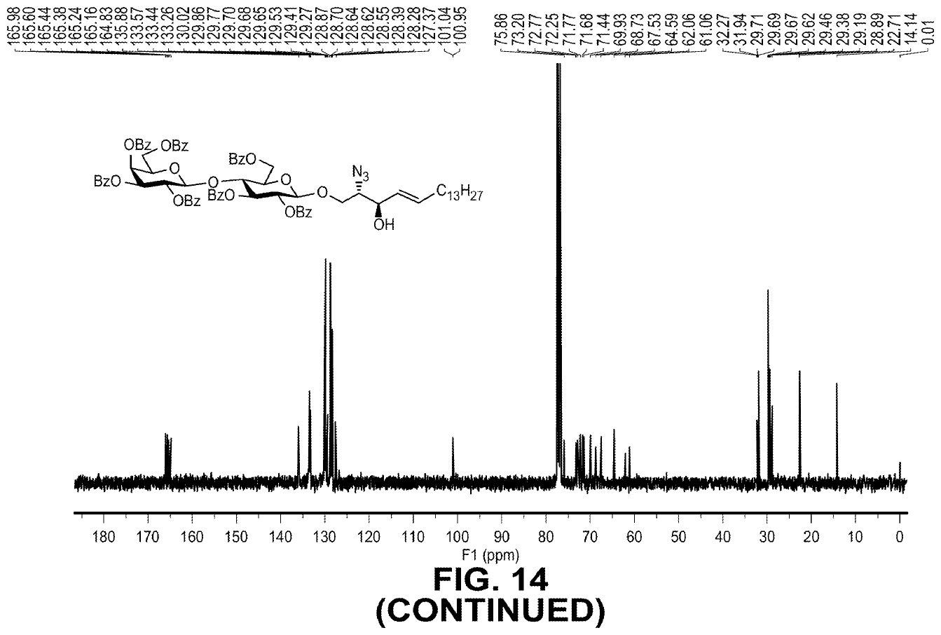

To a solution of II-7b (2 g, 6.14 mmol, 1 eq.) and per-O-benzoylated lactosyl trichloroacetimidate (II-9) (11.2 g, 9.2 mmol, 1.5 eq.) in dry 90 mL of CH₂Cl₂, powdered molecular sieves (4 Å) was added. The mixture was stirred under argon at room temperature for 30 min. The reaction mixture was cooled down to −10° C. and TMSOTf (166 μL, 0.92 mmol, 0.1 eq.) was added. The reaction mixture was then stirred at −10° C. for 2 hours. The reaction was quenched with Et₃N, and the solid was filtered off. The filtrate was concentrated under vacuum, and the residue was purified by a silica gel column chromatography using toluene:EtOAc=10:1 (by volume) as an eluent to produce compound II-10b (5.5 g) as an amorphous solid in 65% yield. ¹H NMR (400 MHz, CDCl₃) δ 8.04-7.16 (m, 35H), 5.83 (t, J=9.4 Hz, 1H), 5.77-5.73 (m, 2H), 5.58-5.35 (m, 4H), 4.92 (d, J=8.0 Hz, 1H), 4.74 (d, J=8.0 Hz, 1H), 4.71-4.65 (dd, J=12.3, 2.0 Hz, 1H), 4.55-4.48 (dd, J=12.2, 4.2 Hz, 1H), 4.29 (t, J=9.5 Hz, 1H), 4.13-4.07 (dt, J=7.4, 5.2 Hz, 1H), 4.02-3.87 (m, 2H), 3.86-3.85 (m, 1H), 3.80-3.77 (m, 2H), 3.65-3.61 (dd, J=10.5, 4.9 Hz, 1H), 3.51-3.47 (q, J=5.4 Hz, 1H), 1.94-1.92 (m, 2H), 1.67-1.65 (m, 3H), 1.33-1.24 (m, 20H), 0.91 (t, J=6.0 Hz, 3H); ¹³C NMR (100 MHz, CDCl₃) δ 166.0, 165.6, 165.4, 165.4, 165.3, 165.2, 164.8, 135.9, 133.6, 133.4, 133.3, 130.0, 129.9, 129.8, 129.7, 129.68, 129.65, 129.5, 129.4, 129.3, 128.9, 128.7, 128.64, 128.62, 128.5, 128.4, 128.3, 127.4, 101.0, 100.9, 75.9, 73.2, 72.8, 72.2, 71.8, 71.7, 71.4, 69.9, 68.7, 67.5, 64.6, 62.1, 61.1, 32.3, 31.9, 29.7, 29.69, 29.67, 29.6, 29.5, 29.4, 29.2, 28.9, 22.7, 14.1. See FIG. 14 for ¹H and ¹³C NMR spectra of compound II-10b.

### O-(β-D-Galactopyranosyl)-(1→4)-(β-D-glucopyranosyl)-(1→1)-(2S, 3R, 4E)-2-amino-octadec-4-ene, LacβSph (d18:1) (II-11b)

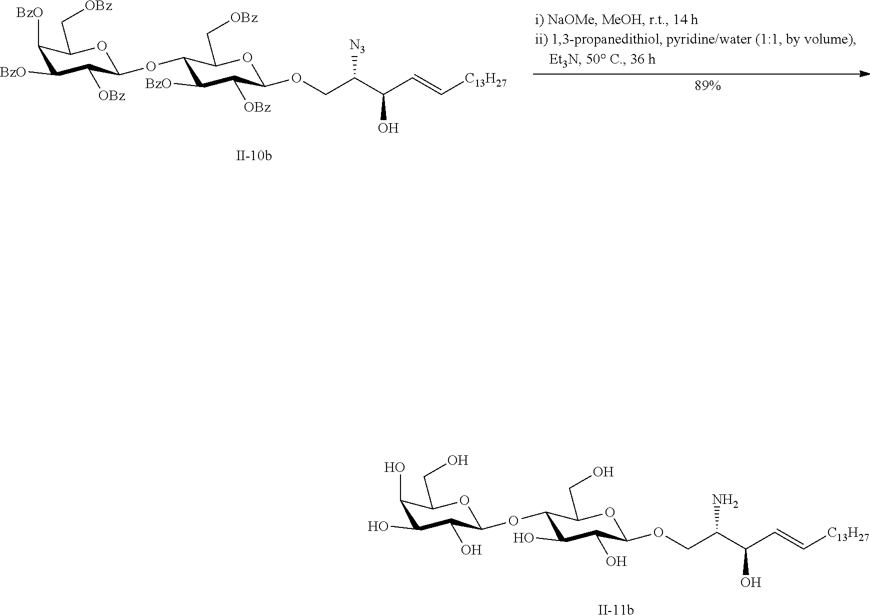

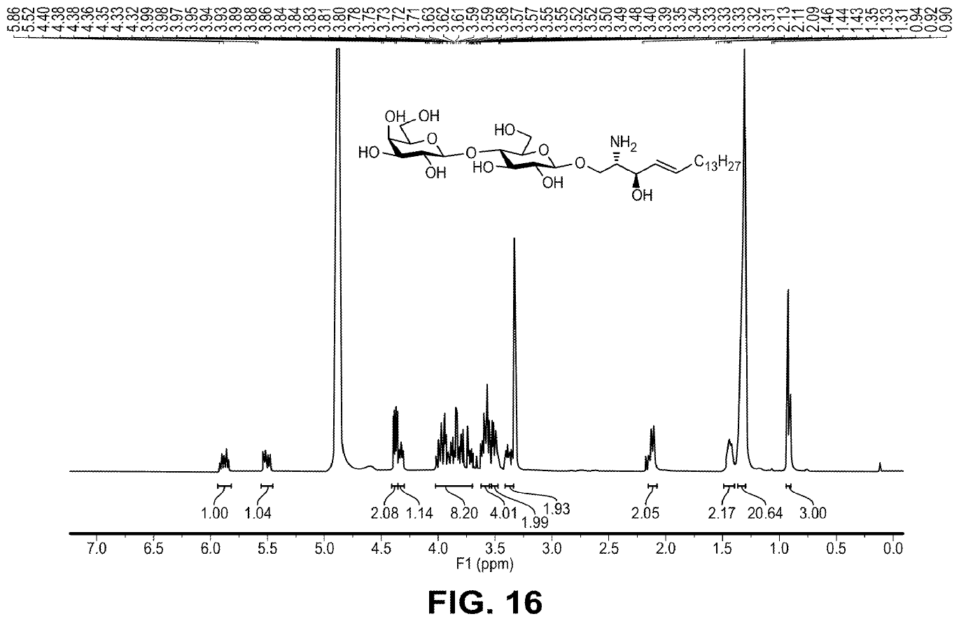

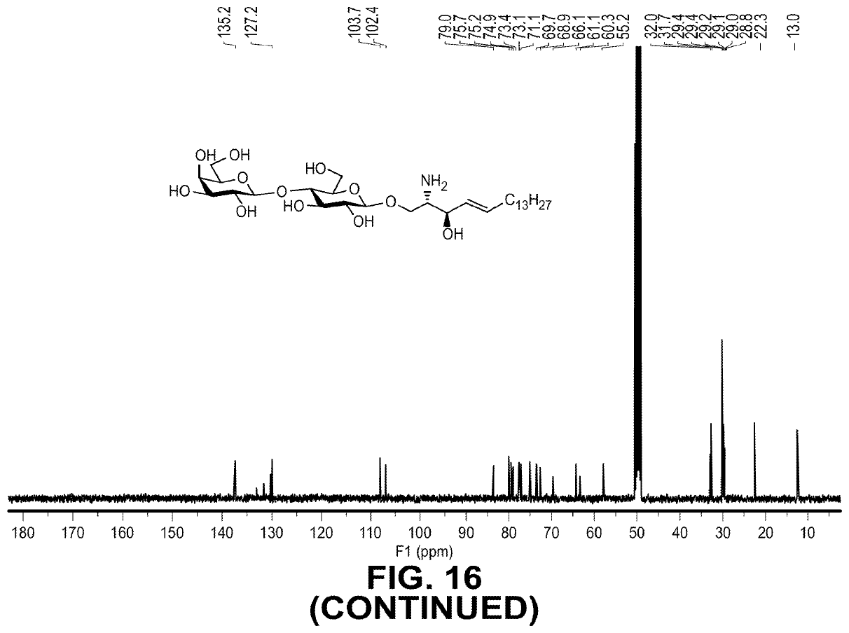

To a solution of II-10b (5 g, 3.6 mmol) in dry MeOH (150 mL), 30% NaOMe in MeOH (6 mL) was added. After being stirred at room temperature for 14 hours, the reaction mixture was neutralized with Dowex® R 50W (H⁺), filtered, and concentrated under a reduced pressure. This intermediate was used in the next step without further purification. To the dry intermediate in 10 mL of pyridine:water=1:1 (by volume), 1,3-propanedithiol (3.7 mL, 36.0 mmol, 10 eq.) and Et₃N (5 mL) were added and the mixture was stirred at 50° C. for 36 hours. The reaction mixture was concentrated and purified by a silica gel column chromatography using chloroform:methanol:water=5:4:1 (by volume) as an eluent to produce LacPSph (d18:1) (II-11b) (2 g) as a white amorphous powder in 89% yield. ¹H NMR (400 MHz, CD₃OD) δ 5.94-5.82 (dt, J=16.0, 6.8 Hz, 1H), 5.56-5.46 (ddd, J=15.4, 6.8, 1.6 Hz, 1H), 4.41-4.36 (dd, J=7.7, 4.7 Hz, 2H), 4.36-4.31 (t, J=5.9 Hz, 1H), 4.03-3.70 (m, 8H), 3.63-3.55 (m, 4H), 3.53-3.48 (dt, J=9.4, 3.6 Hz, 2H), 3.42-3.34 (m, 2H), 2.15-2.08 (t, J=7.2 Hz, 2H), 1.49-1.40 (t, J=7.2 Hz, 2H), 1.37-1.30 (d, J=7.5 Hz, 21H), 0.92 (m, 3H); ¹³C NMR (100 MHz, CD₃OD) δ 135.2, 127.2, 103.7, 102.4, 79.0, 75.7, 75.2, 74.9, 73.4, 73.1, 71.1, 69.7, 68.9, 66.1, 61.1, 60.3, 55.2, 32.0, 31.7, 29.5, 29.4, 29.2, 29.1, 29.0, 28.8, 22.3, 13.0. See FIG. 16 for ¹H and ¹³C NMR spectra of compound II-11b.

### Synthesis of O-(2,3,4,6-tetra-O-benzoyl-β-D-galactopyranosyl)-(1→4)-(2,3,6-tri-O-benzoyl-β-D-glucopyranosyl)-(1→1)-(2S, 3R, 4E)-2-azido-3-O-benzoyl-octadec-4-ene (II-12b)

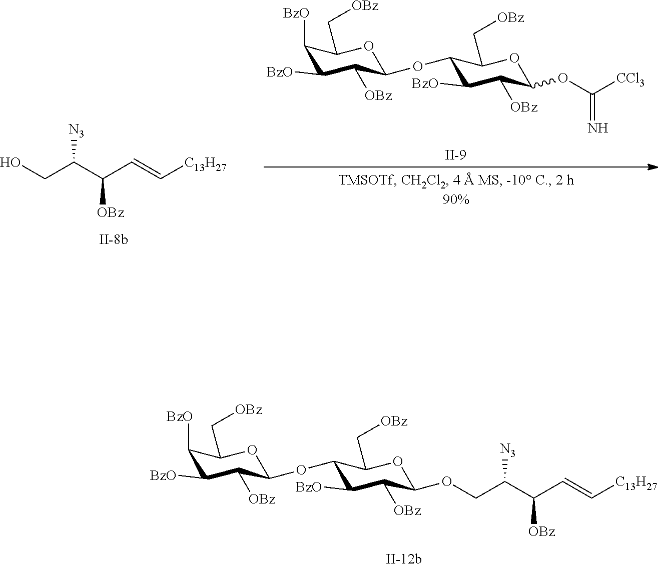

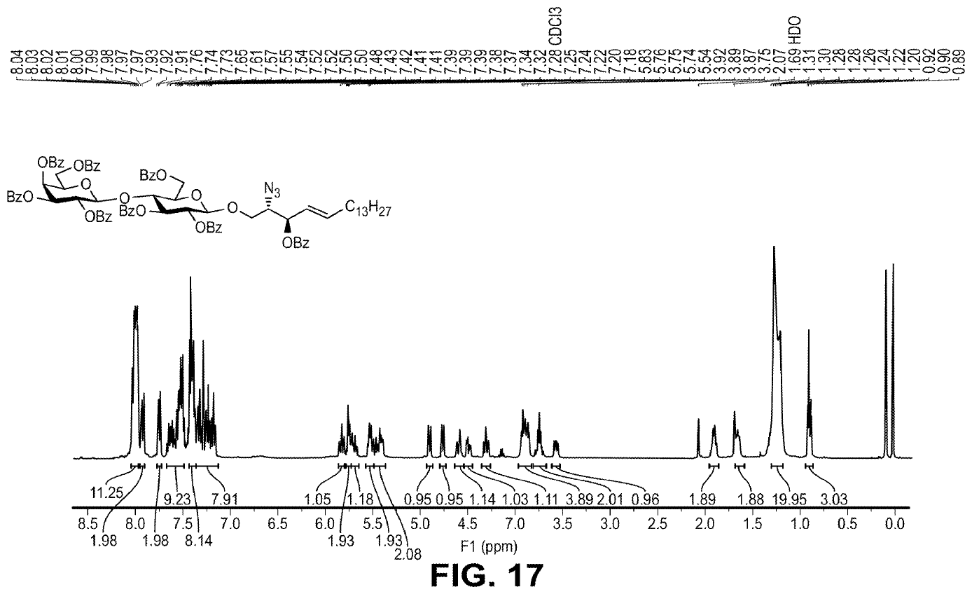

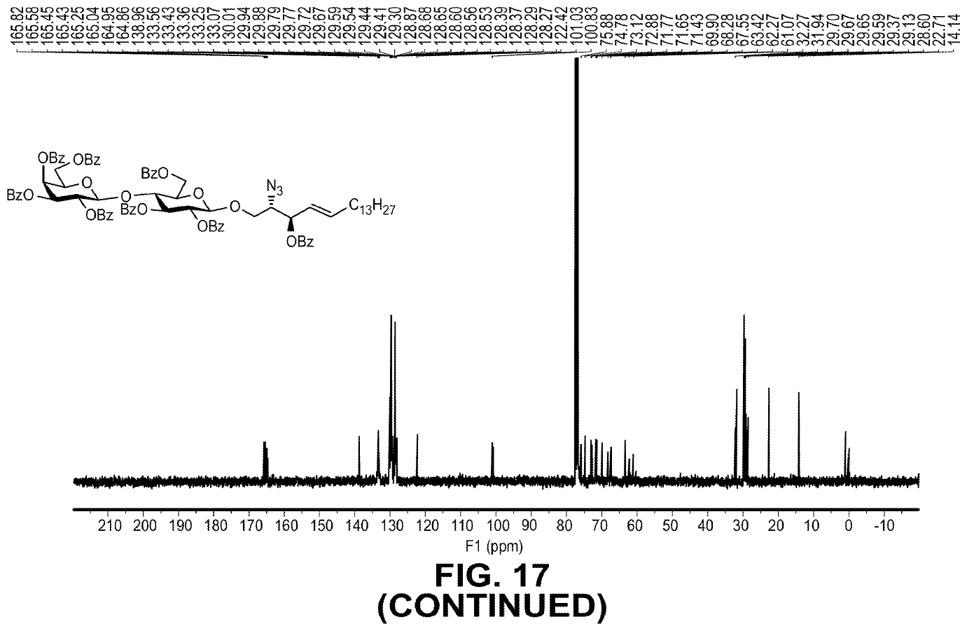

To a solution of I-8b (2 g, 4.65 mmol, 1 eq.) and per-O-benzoylated lactosyl trichloroacetimidate (II-9) (7.35 g, 6.05 mmol, 1.3 eq.) in 40 mL of dry CH₂Cl₂, powdered molecular sieves (4 Å) was added. The mixture was stirred under argon at room temperature for 30 minutes. The reaction mixture was cooled down to −10° C. and TMSOTf (110 μL, 0.60 mmol, 0.1 eq.) was added. The reaction mixture was then stirred at −10° C. for 2 hours. The reaction was quenched with Et₃N, and the solid was filtered off. The filtrate was concentrated under vacuum, and the residue was purified by a silica gel column chromatography using hexane:EtOAc=4:1 (by volume) as an eluent to produce compound II-12b (6.2 g) as an amorphous solid in 90% yield. ¹H NMR (400 MHz, CDCl₃) δ 8.05-7.16 (m, 40H), 5.83 (t, J=9.3 Hz, 1H), 5.76-5.72 (m, 2H), 5.72-5.67 (m, 1H), 5.55-5.49 (m, 2H), 5.47-5.39 (m, 2H), 4.89 (d, J=7.9 Hz, 1H), 4.75 (d, J=7.7 Hz, 1H), 4.62-4.58 (dd, J=12.3, 1.9 Hz, 1H), 4.47-4.51 (dd, J=12.2, 4.3 Hz, 1H), 4.31 (t, J=10.0 Hz, 1H), 3.85-3.95 (m, 4H), 3.79-3.72 (m, 2H), 3.59-3.55 (dd, J=9.9, 5.5 Hz, 1H), 1.93-1.88 (m, 2H), 1.67-1.64 (m, 2H), 1.31-1.20 (m, 7.3 Hz, 20H), 0.90 (t, J=6.0 Hz, 3H); ¹³C NMR (100 MHz, CDCl₃) δ 165.8, 165.6, 165.5, 165.4, 165.2, 165.0, 165.49, 164.9, 139.0, 133.6, 133.4, 133.3, 133.2, 133.1, 130.0, 129.9, 129.8, 129.88, 129.8, 129.79, 129.77, 129.6, 129.5, 129.44, 129.41, 129.3, 128.9, 128.7, 128.65, 128.60, 128.56, 128.53, 128.4, 128.39, 128.37, 128.2, 122.4, 101.0, 100.8, 75.9, 74.8, 73.1, 72.9, 71.8, 71.6, 71.4, 69.9, 68.3, 67.5, 63.4, 62.3, 61.1, 32.3, 31.9, 29.7, 29.67, 29.65, 29.6, 29.4, 29.1, 28.6, 22.7, 14.1. See FIG. 17 for ¹H and ¹³C NMR spectra of compound II-12b.

### O-(β-D-Galactopyranosyl)-(1→4)-(β-D-glucopyranosyl)-(1→1)-(2S, 3R, 4E)-2-amino-octadec-4-ene, LacβSph (d18:1) (II-11b)

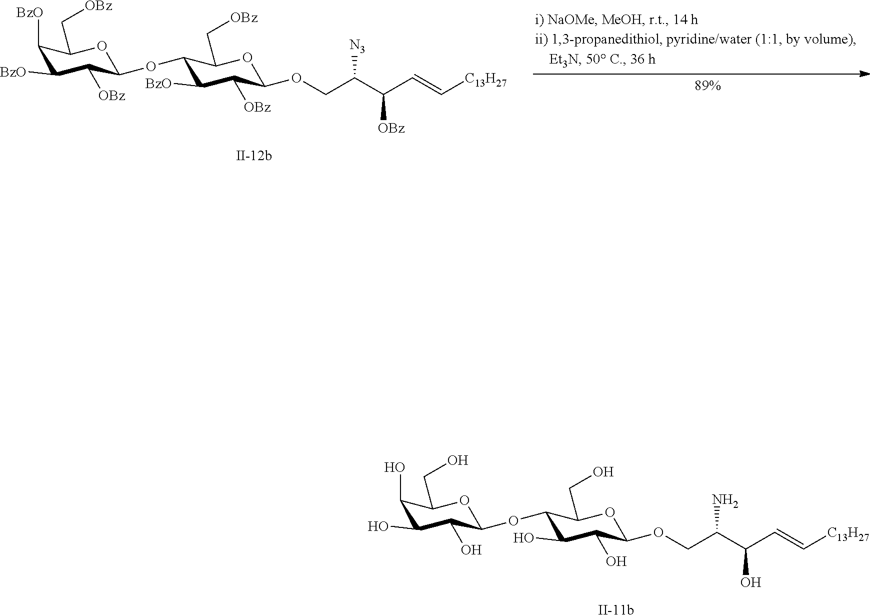

To a solution of II-12b (6 g, 4.0 mmol) in dry MeOH (150 mL), 30% NaOMe in MeOH (5 mL) was added. After being stirred at room temperature for 14 hours, the reaction mixture was neutralized with Dowex® 50W (He), filtered, and concentrated under a reduced pressure. This intermediate was used in the next step without further purification. To the dry intermediate in 10 mL of pyridine:water=1:1 (by volume), 1,3-propanedithiol (4.1 mL, 40.0 mmol, 10 eq.) and Et₃N (6 mL) were added and the mixture was stirred at 50° C. for 36 hours. The reaction mixture was concentrated and purified by a silica gel column chromatography using chloroform:methanol:water=5:4:1 (by volume) as an eluent to produce LacPSph (d18:1) (II-11b) (2.24 g) as a white amorphous powder in 89% yield.

### Synthesis of O-(β-D-glucopyranosyl)-(1→1)-(2S, 3R, 4E)-2-amino-icos-4-ene, GlcβSph (d20:1) (II-15)

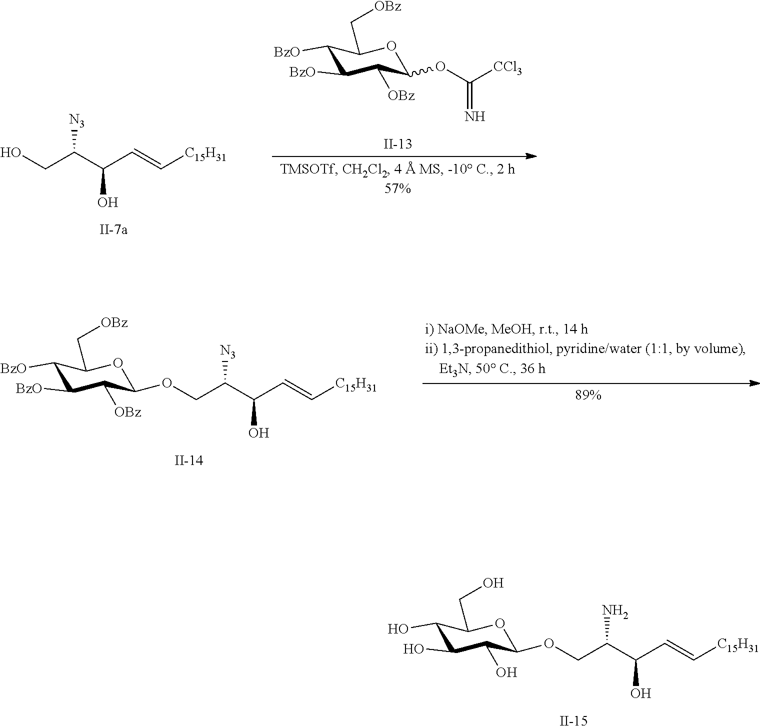

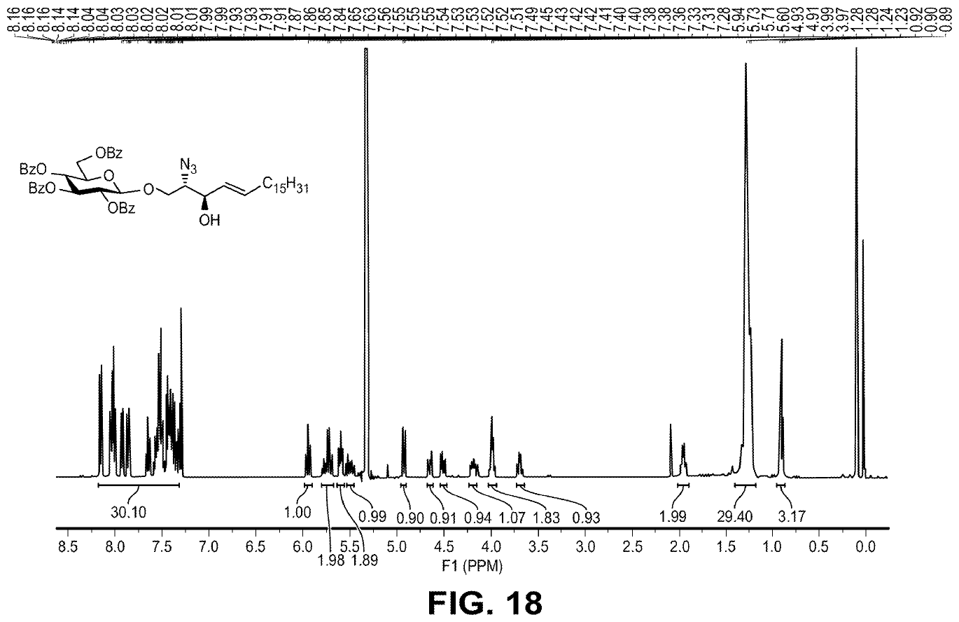

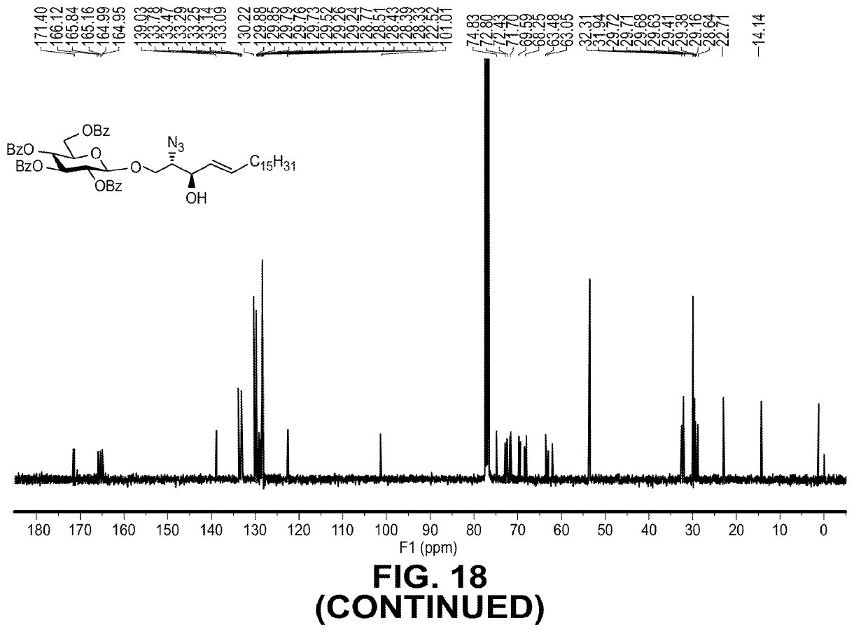

To a solution of I-7a (500 mg. 1.41 mmol, 1 eq.) and per-O-benzoylated glucosyl trichloroacetimidate 11-13 (1.25 g, 2.82 mmol, 1.2 eq.) in 10 mL of dry CH₂Cl₂, powdered molecular sieves (4 Å) was added. The mixture was stirred under argon at room temperature for 30 min. The reaction mixture was cooled down to −10° C. and TMSOTf (51 μL. 0.28 mmol, 0.1 eq.) was added. The reaction mixture was then stirred at −10° C. for 2 hours. The reaction was quenched with Et₃N, and the solid was filtered off. The filtrate was concentrated under vacuum, and the residue was purified by a silica gel column chromatography using hexane:EtOAc=4:1 (by volume) as an eluent to produce compound II-14 (751 mg) as an amorphous solid in 57% yield. ¹H NMR (400 MHz, CDCl₃) δ 8.16-7.28 (m, 30H), 5.94 (t, J=8.0 Hz, 1H), 5.77-5.69 (m, 2H), 5.61-5.59 (m, 2H), 5.58-5.48 (m, 1H), 4.91 (d, J=7.8 Hz, 1H), 4.66-4.63 (dd, J=12.2, 3.2 Hz, 1H), 4.53-4.48 (dd, J=12.2, 5.2 Hz, 1H), 4.21-4.14 (m, 1H), 4.01-3.96 (m, 2H), 3.69-3.66 (m, 1H), 1.95-1.94 (m, 2H), 1.28-1.23 (m, 26H), 0.90 (t, J=6.0 Hz, 3H); ¹³C NMR (100 MHz, CDCl₃) δ 171.4, 166.1, 165.8, 165.2, 165.0, 164.9, 139.0, 133.8, 133.5, 133.29, 133.25, 133.14, 133.09, 130.2, 130.0, 129.9, 129.8, 129.75, 129.7, 129.6, 129.5, 129.3, 129.2, 128.8, 128.5, 128.4, 128.3, 128.2, 122.5, 101.0, 74.8, 72.8, 72.4, 71.7, 69.6, 68.3, 63.5, 63.1, 32.3, 31.9, 29.73, 29.71, 29.68, 29.6, 29.41, 29.38, 29.2, 28.6, 22.7, 14.1. See FIG. 18 for ¹H and ¹³C NMR spectra of compound H₁-14.

To a solution of II-14 (750 mg, 0.80 mmol) in 20 mL of dry MeOH, 30% NaOMe in MeOH (2 mL) was added. After being stirred at room temperature for 14 hours, the reaction mixture was neutralized with Dowex® 50W (H′), filtered and concentrated under a reduced pressure. This intermediate was used in the next step without further purification. To the dry intermediate in 4 mL of pyridine:water=1:1 (by volume), 1,3-propanedithiol (0.8 mL, 8.0 mmol, 10 eq.) and Et₃N (1 mL) were added and the mixture was stirred at 50° C. for 36 hours.

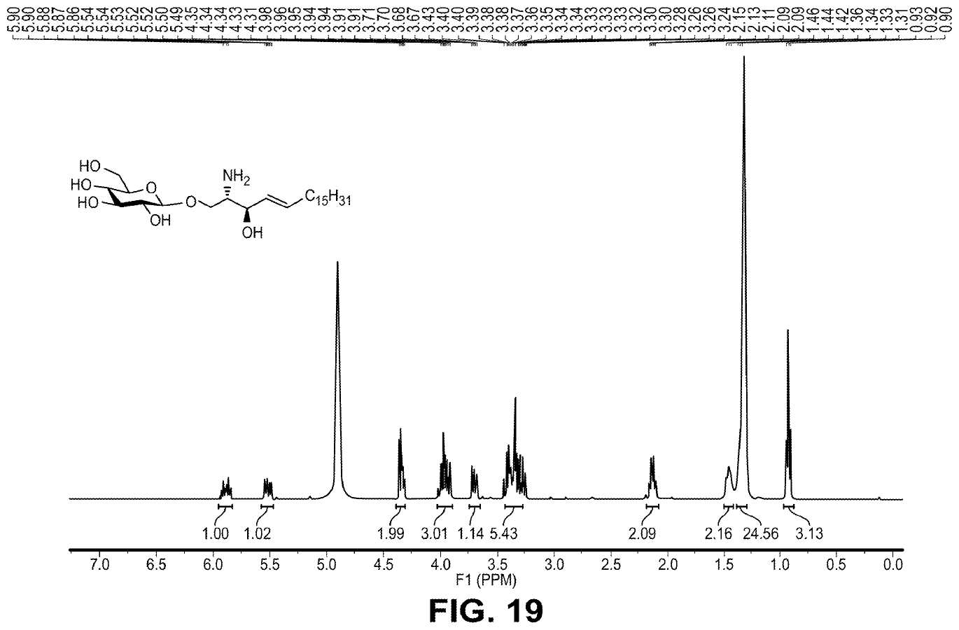

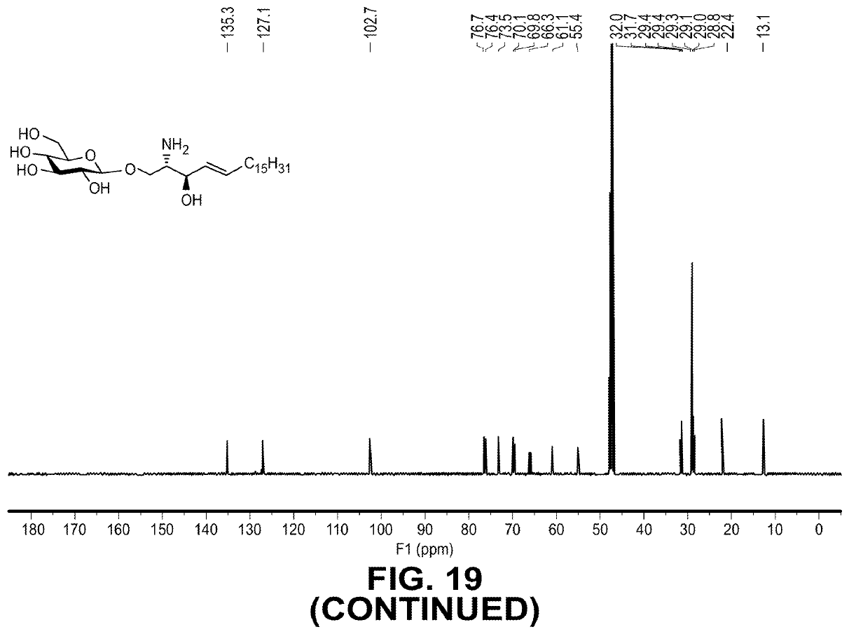

The reaction mixture was concentrated and purified by a silica gel column chromatography using chloroform:methanol:water=10:5:1 (by volume) as an eluent to produce GlcβSph (d20:1) (II-15) (350 mg) as a white amorphous powder in 89% yield. ¹H NMR (400 MHz, CD₃OD) δ 5.92-5.85 (dtd, J=15.0, 6.8, 1.1 Hz, 1H), 5.55-5.48 (ddt, J=15.4, 6.9, 1.5 Hz, 1H), 4.35-4.31 (m, 2H), 4.98-3.91 (m, 3H), 3.71-3.67 (dd, J=11.8, 5.7 Hz, 1H), 3.45-3.24 (m, 5H), 2.15-2.09 (m, 2H), 1.46-1.42 (m, 2H), 1.36-1.31 (m, 24H), 0.92 (t, J=6.0 Hz, 3H); ¹³C NMR (100 MHz, CD₃OD) δ 135.3, 127.1, 102.7, 76.7, 76.4, 73.5, 70.1, 69.8, 66.3, 61.1, 55.4, 32.0, 31.7, 29.4, 29.3, 29.2, 29.1, 29.0, 28.8, 22.4, 13.1. See FIG. 19 for ¹H and ¹³C NMR spectra of compound II-15.

### Synthesis of O-(β-D-galactopyranosyl)-(1→1)-(2S, 3R, 4E)-2-amino-icos-4-ene, GalβSph (d20:1) (II-18)

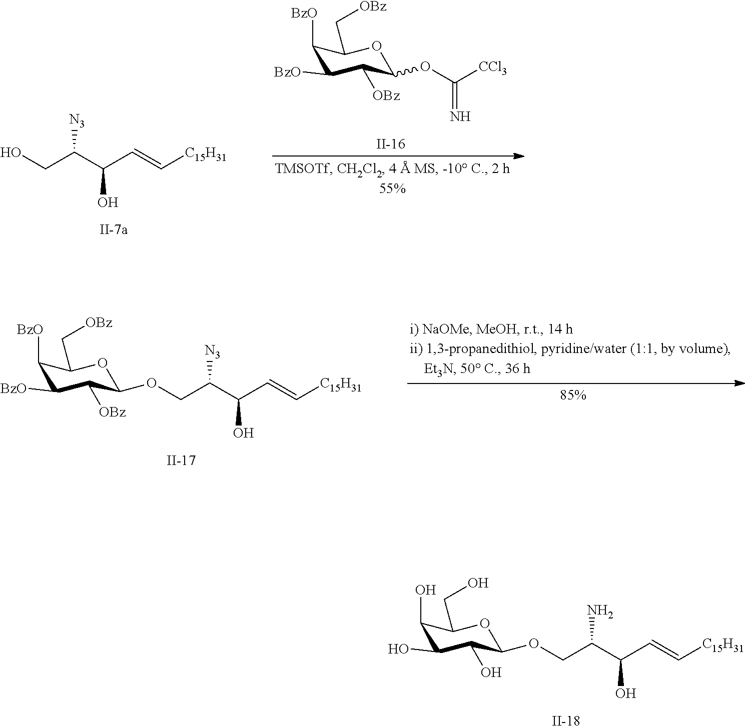

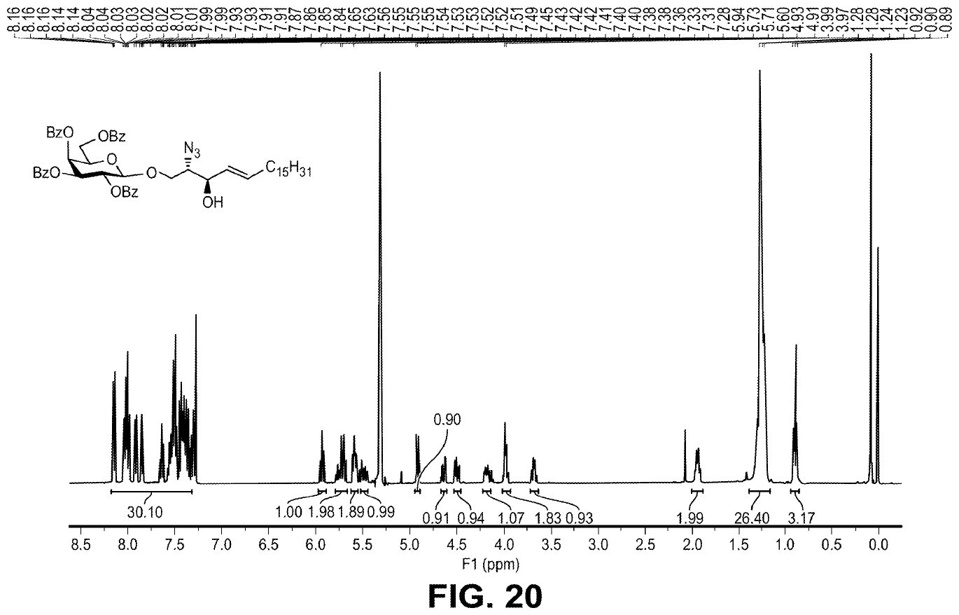

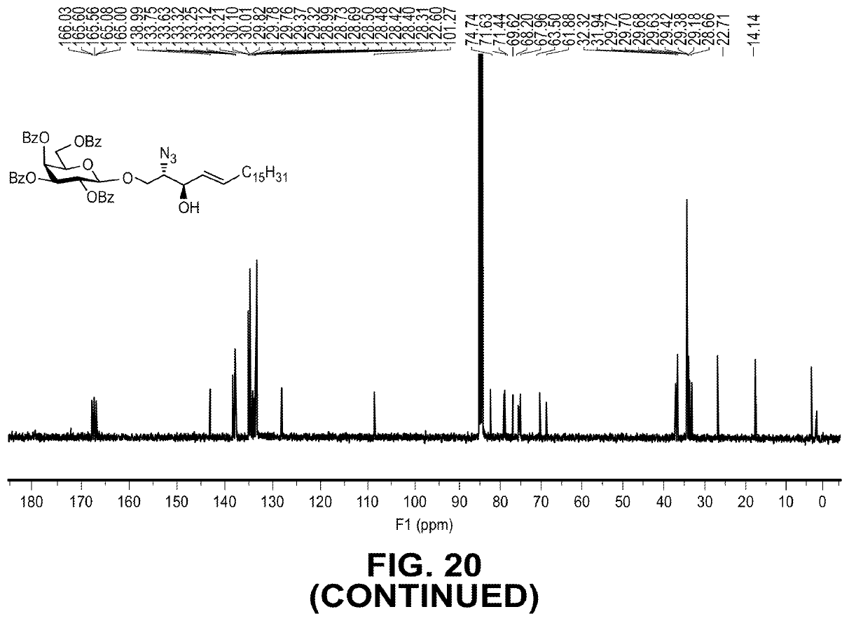

To a solution of II-7a (500 mg, 1.41 mmol, 1 eq.) and per-O-benzoylated galactosyl trichloroacetimidate 11-16 (1.25 g, 2.82 mmol, 1.2 eq.) in 10 mL of dry CH₂Cl₂, powdered molecular sieves (4 Å) was added. The mixture was stirred under argon at room temperature for 30 minutes. The reaction mixture was cooled down to −10° C. and TMSOTf (51 μL, 0.28 mmol, 0.1 eq.) was added. The reaction mixture was then stirred at −10° C. for 2 hours. The reaction was quenched with Et₃N, and the solid was filtered off. The filtrate was concentrated under vacuum, and the residue was purified by a silica gel column chromatography using hexane:EtOAc=4:1 (by volume) as an eluent to produce compound II-17 (724 mg) as an amorphous solid in 55% yield. ¹H NMR (400 MHz, CDCl₃) δ 8.16-7.25 (m, 30H), 6.01 (d, J=3.4 Hz, 1H), 5.87-5.83 (dd, J=10.4, 7.9 Hz, 1H), 5.80-5.72 (dt, J=15.4, 6.7 Hz, 1H), 5.66-5.61 (m, 2H), 5.54-5.48 (m, 1H), 4.88 (d, J=8.0 Hz, 1H), 4.67-4.63 (dd, J=11.0, 6.1 Hz, 1H), 4.42-4.33 (m, 2H), 4.08-4.02 (m, 2H), 3.74-3.70 (dd, J=9.5, 4.9 Hz, 1H), 1.96-1.93 (m, 2H), 1.27-1.24 (m, 26H), 0.90 (t, J=6.0 Hz, 3H); ¹³C NMR (100 MHz, CDCl₃) δ, 166.0, 165.6, 165.5, 165.1, 165.0, 139.0, 133.7, 133.6, 133.3, 133.2, 133.1, 130.2, 130.1, 130.0, 129.8, 129.78, 129.76, 129.4, 129.3, 129.0, 128.7, 128.6, 128.5, 128.4, 128.35, 128.3, 128.2, 122.6, 101.3, 74.7, 71.6, 71.4, 69.6, 68.2, 68.0, 63.5, 61.9, 32.3, 31.9, 29.72, 29.70, 29.68, 29.63, 29.4, 29.3, 29.2, 28.7, 22.7, 14.1. See FIG. 20 for ¹H and ¹³C NMR spectra of compound II-17.

To a solution of 11-17 (700 mg, 0.75 mmol) in 10 mL of dry MeOH, NaOMe was added. After being stirred at room temperature for 14 hours, the reaction mixture was neutralized with Dowex® 50W (H⁺), filtered and concentrated under reduced pressure. This intermediate was used in the next step without further purification. To the dry intermediate in 4 mL of pyridine:water=1:1 (by volume), 1,3-propanedithiol (750 μL, 7.5 mmol, 10 eq.) and Et₃N (1 mL) were added and the mixture was stirred at 50° C. for 36 hours. The reaction mixture was concentrated and purified by a silica gel column chromatography using chloroform:methanol:water=10:5:1 (by volume) as an eluent to produce GalβSph (d20:1) (II-18) (312 mg) as a white amorphous powder in 85% yield. ¹H NMR (400 MHz, CD₃OD) δ 5.90-5.86 (m, 1H), 5.54-5.49 (dd, J=15.4, 6.8 Hz, 1H), 4.34-4.29 (m, 2H), 3.97-3.95 (m, 2H), 3.85 (d, J=3.0 Hz, 1H), 3.82-3.73 (m, 2H), 3.60-3.50 (m, 3H), 3.38-3.36 (m, 1H), 3.34-3.32 (m, 1H), 2.15-2.09 (q, J=7.2 Hz, 2H), 1.46-1.41 (m, 2H), 1.37-1.28 (m, 24H). 0.92 (t, J=6.0 Hz, 3H); ¹³C NMR (100 MHz, CD₃OD) δ 135.2, 127.2, 103.2, 75.6, 73.3, 71.1, 69.8, 68.9, 66.2, 61.2, 55.6, 32.0, 31.7, 29.4, 29.3, 29.2, 29.1, 29.0, 28.8, 22.3, 13.1.

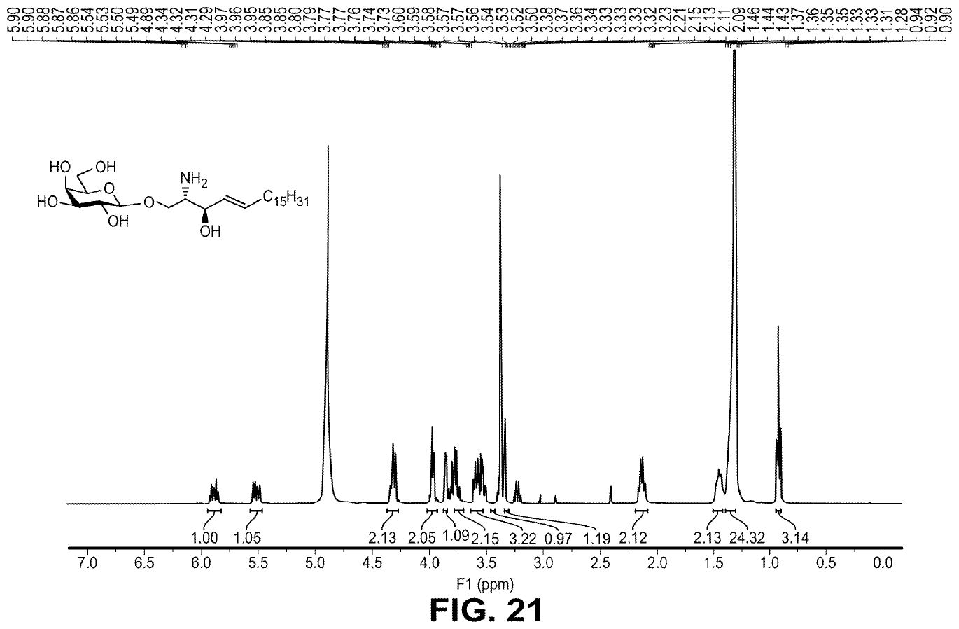

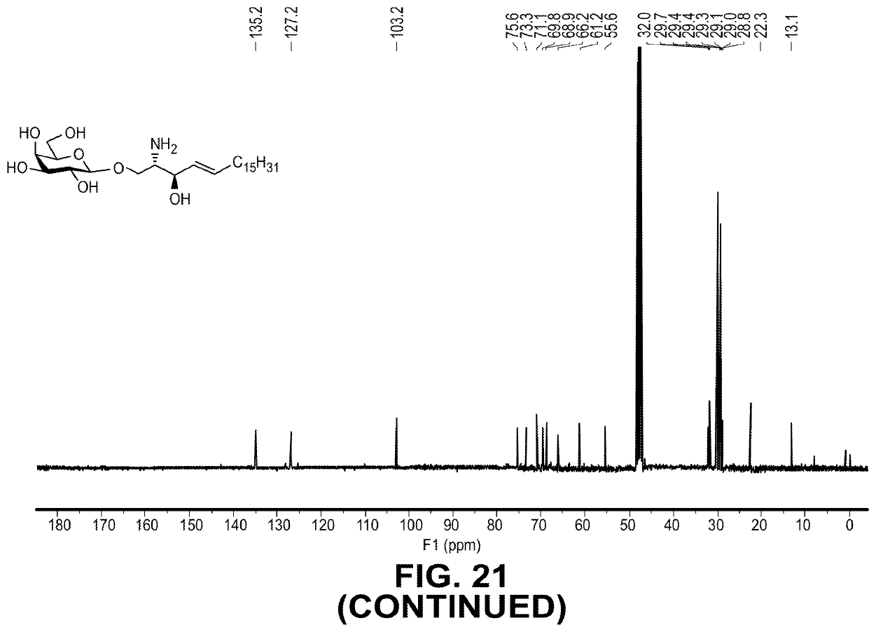

See FIG. 21 for ¹H and ¹³C NMR spectra of compound II-18.

### Synthesis of O-(3-SO₃H-β-D-galactopyranosyl)-(1→1)-(2S, 3R, 4E)-2-amino-icos-4-ene (II-21)

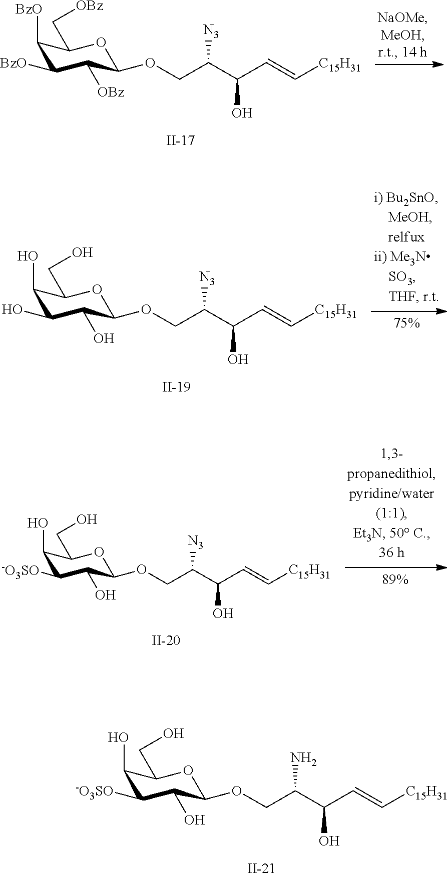

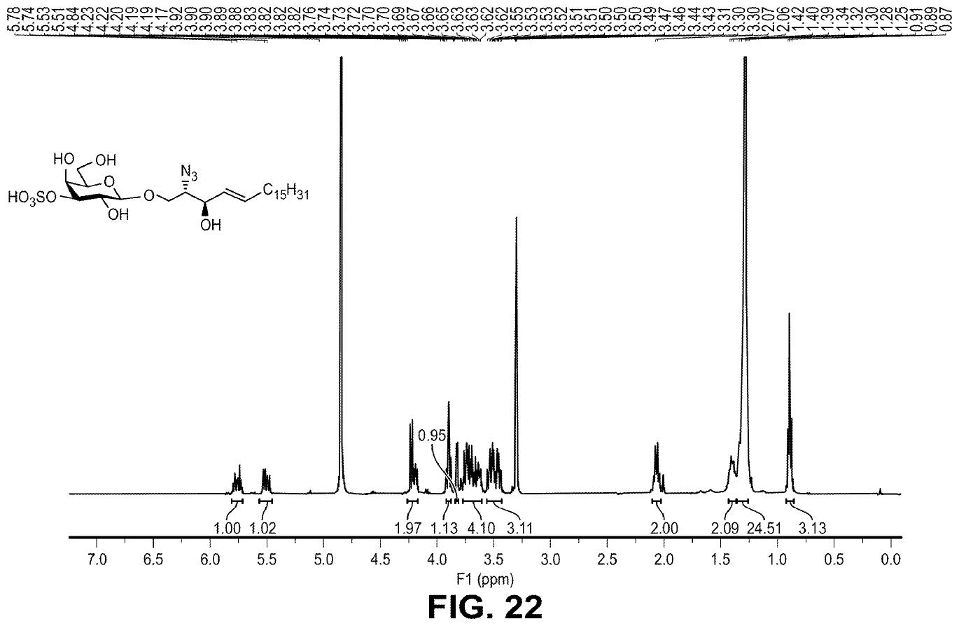

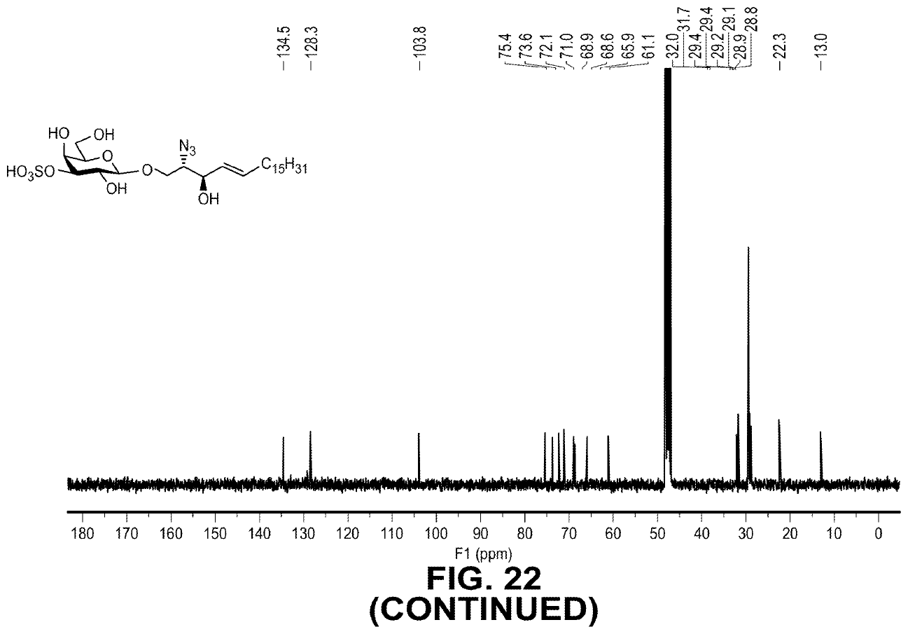

To a solution of II-17 (500 mg, 0.54 mmol) in 10 mL of dry MeOH, 30% NaOMe in MeOH (2 mL) was added. After being stirred at room temperature for 14 hours, the reaction mixture was neutralized with Dowex® 50W (H⁺), filtered and concentrated under reduced pressure. The crude was purified by a silica gel column chromatography using CH₂Cl₂:MeOH=5:1 (by volume) as an eluent to produce compound II-19. The obtained compound II-19 was dissolved in 10 mL of MeOH (dry). Bu₂SnO (200 mg, 0.81 mmol, 1.5 eq.) was added and the mixture was heated at 80° C. for 2 hours. The MeOH was evaporated and the obtained crude was dissolved in 8 mL of anhydrous THF. Me₃N·SO₃ (114 mg, 0.81 mmol, 1.5 eq.) was added and the mixture was stirred at room temperature for 8 hours. The organic layer was evaporated and the obtained crude was purified on a silica gel column chromatography using CH₂Cl₂:MeOH=3:1 (by volume) as an eluent to produce compound II-20 (235 mg) as a white solid in 75% yield. ¹H NMR (400 MHz, CD₃OD) δ 5.78-5.74 (m, 1H), 5.53-5.47 (dd, J=15.4, 7.5 Hz, 1H), 4.23-4.17 (m, 2H), 3.92-3.88 (m, 1H), 3.83-3.82 (m, 1H), 3.77-3.62 (m, 4H), 3.55-3.43 (m, 3H), 2.07-2.06 (m, 2H), 1.42-1.39 (m, 2H), 1.34-1.25 (m, 24H), 0.89 (t, J=6.0 Hz, 3H); ¹³C NMR (100 MHz, CD₃OD) δ 134.5, 128.3, 103.8, 75.4, 73.6, 72.1, 71.0, 68.9, 68.6, 65.9, 61.1, 32.0, 31.7, 29.4, 29.3, 29.2, 29.1, 28.9, 28.8, 22.3, 13.0. See FIG. 22 for ¹H and ¹³C NMR spectra of compound II-20.

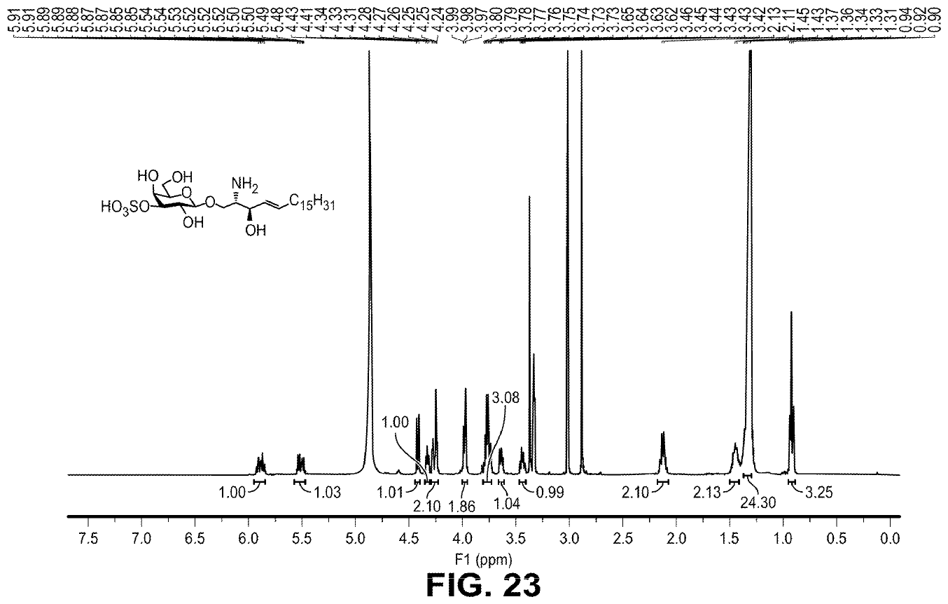

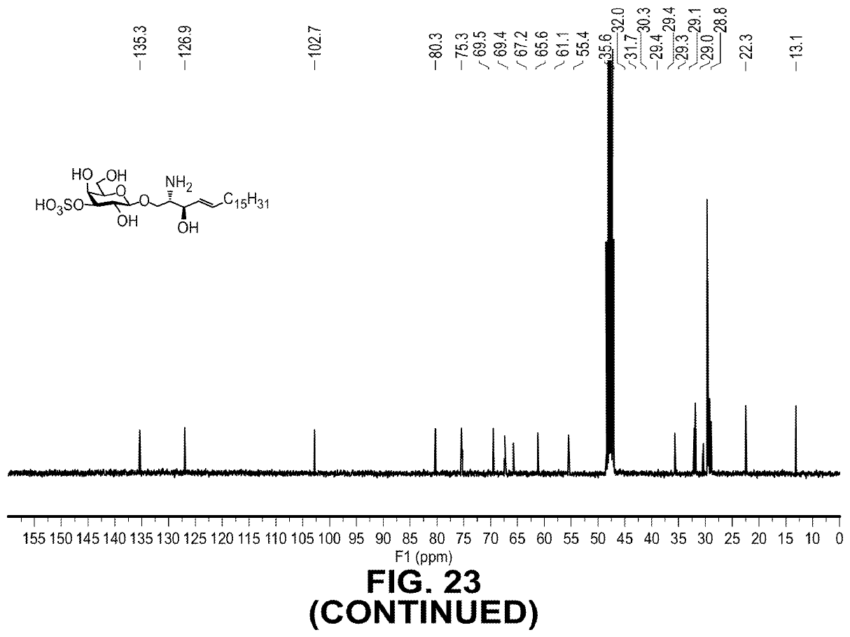

To a solution of compound 1H-20 (200 mg. 0.34 mmol) in 3 mL of pyridine:water=1:1 (by volume), 1,3-propanedithiol (336 μL, 3.4 mmol, 10 eq.) and Et₃N (1 mL) were added and the mixture was stirred at 50° C. for 36 hours. The reaction mixture was concentrated and purified by a silica gel column chromatography using CH₂Cl₂:methanol:water=20:5:1 (by volume) as an eluent to produce 3-SO₃H-GalβSph (d20:1) (II-21) (170 mg) as a white amorphous powder in 89% yield. ¹H NMR (400 MHz, CD₃OD) δ 5.91-5.85 (ddd, J=13.5, 7.5, 1.2 Hz, 1H), 5.54-5.48 (ddt, J=15.4, 6.8, 1.5 Hz, 1H), 4.41 (d, J=8.0 Hz, 1H), 4.33 (t, J=8.0 Hz, 1H), 4.28-4.24 (m, 2H), 3.99-3.97 (m, 2H), 3.80-3.73 (m, 3H), 3.65-3.62 (m, 1H), 3.46-3.42 (m, 1H), 2.13-2.11 (m, 2H), 1.45-1.43 (m, 2H), 1.37-1.31 (m, 24H), 0.92 (t, J=6.0 Hz, 3H); ¹C NMR (100 MHz, CD₃OD) δ 135.3, 126.9, 102.7, 80.3, 75.3, 69.5, 69.4, 67.2, 65.6, 61.1, 55.4, 35.6, 32.0, 31.7, 30.3, 29.5, 29.4, 29.3, 29.1, 29.0, 28.8, 22.3, 13.1. See FIG. 23 for ¹H and ¹³C NMR spectra of compound II-21.

### Example 3: Synthesis of Sphingosine (d20:1) (II-22a) and Sphingosine (d18:1) (II-22b)

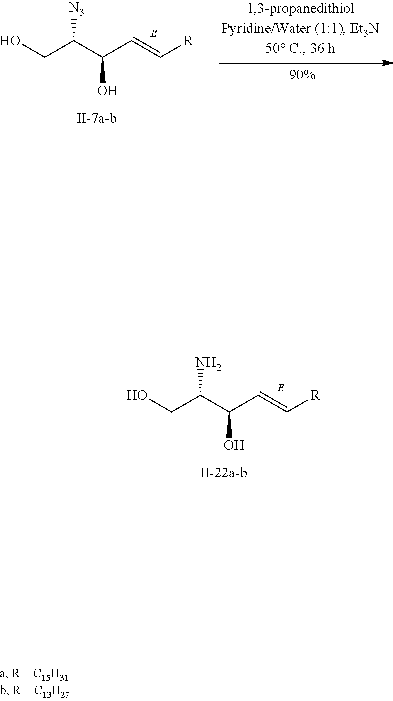

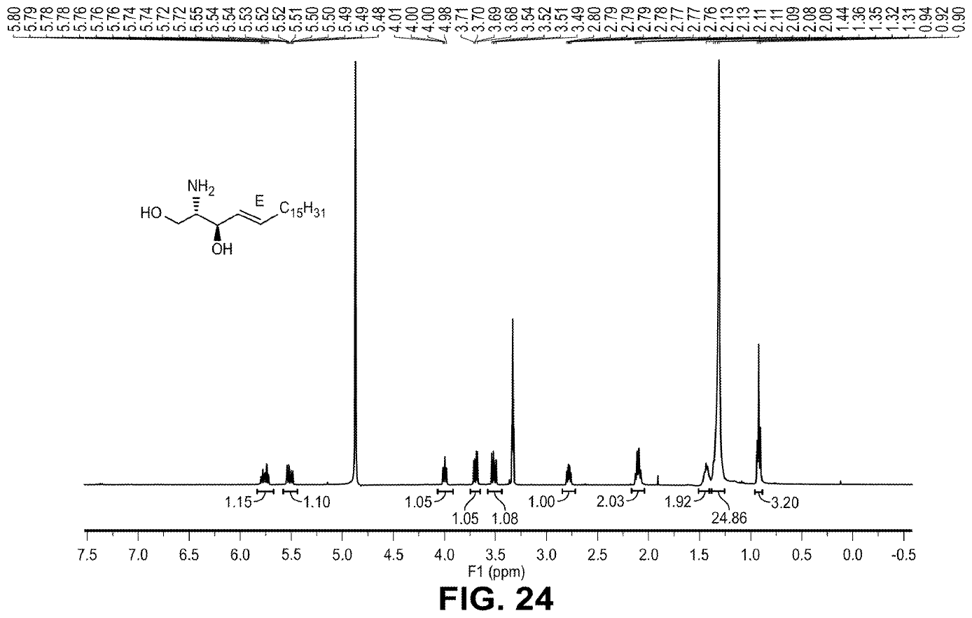

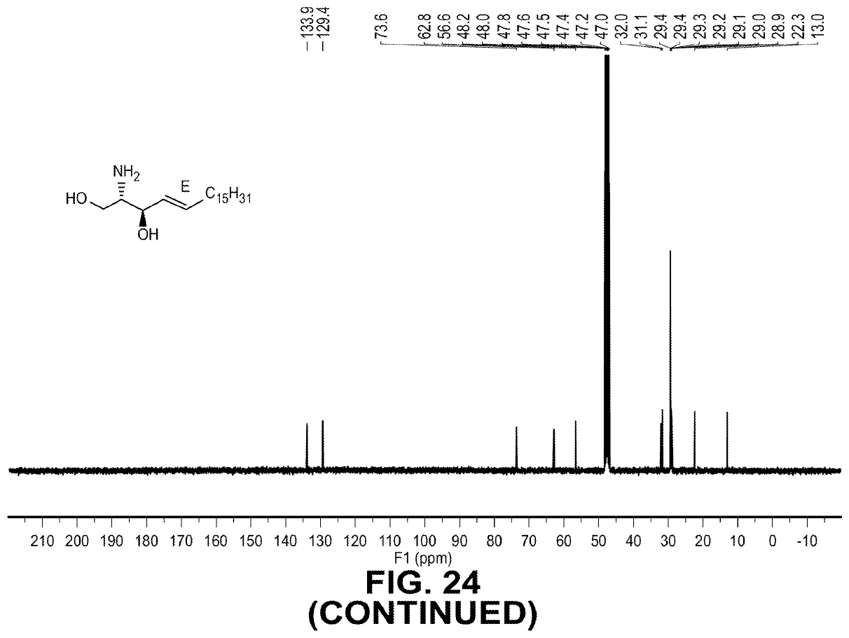

To a solution of compound II-7a (150 mg. 0.42 mmol) in 2 mL of pyridine:water=1:1 (by volume). 1,3-propanedithiol (425 μL, 4.20 mmol. 10 eq.) and Et₃N (150 μL) were added. The resulting mixture was stirred at 50° C. for 36 hours. The reaction mixture was concentrated and purified by a silica gel column chromatography using CH₂Cl₂:methanol=2:1 (by volume) as an eluent to produce sphingosine (d20:1) (II-22a) (125 mg) as a white amorphous powder in 90% yield. ¹H NMR (400 MHz, CD₃OD) δ=5.76 (dtd, J=14.6, 6.7, 1.0 Hz, 1H), 5.60-5.28 (m, 1H), 4.03-3.86 (m, 1H), 3.72-3.66 (dd, J=10.9, 4.5 Hz, 1H), 3.55-3.48 (dd, J=10.9, 7.0 Hz, 1H), 2.81-2.75 (ddd, J=7.0, 6.0, 4.5 Hz, 1H), 2.14-2.08 (m, 2H), 1.46-1.40 (m, 2H), 1.38-0.98 (m, 24H), 0.92 (t, J=6.0 Hz, 3H); ¹³C NMR (100 MHz, CD₃OD) δ 133.9, 129.4, 73.6, 62.8, 56.6, 32.0, 31.7, 29.4, 29.36, 29.3, 29.2, 29.1, 29.0, 28.9, 22.3, 13.0. See FIG. 24 for ¹H and ¹³C NMR spectra of compound II-22a.

To a solution of compound II-7b (150 mg, 0.46 mmol) in 2 mL of pyridine:water=1:1 (by volume), 1,3-propanedithiol (466 μL, 4.60 mmol, 10 eq.) and Et₃N (150 μL) were added. The resulting mixture was stirred at 50° C. for 36 hours. The reaction mixture was concentrated and purified by a silica gel column chromatography using CH₂Cl₂:methanol=2:1 (by volume) as an eluent to produce sphingosine (d18:1) (II-22b) (124 mg) as a white amorphous powder in 90% yield. ¹H NMR (400 MHz, CD₃OD) δ=5.76 (dt, J=15.4, 6.7 Hz, 1H), 5.52 (ddt, J=15.3, 7.3, 1.5 Hz, 1H), 4.0 (t, J=6.7 Hz, 1H), 3.69 (dd, J=10.9, 4.4 Hz, 1H), 3.53 (dd, J=9.0, 7.0 Hz, 1H), 2.78 (dt, J=6.4, 3.2 Hz, 1H), 2.13-2.08 (m, 2H), 1.46-1.43 (m, 2H), 1.40-0.98 (m, 20H), 0.90 (t, J=6.0 Hz, 3H); ¹³C NMR (100 MHz, CD₃OD) δ 133.8, 129.5, 73.7, 62.9, 56.6, 32.1, 31.7, 29.5, 29.4, 29.36, 29.3, 29.2, 29.1, 29.0, 22.4, 13.2.

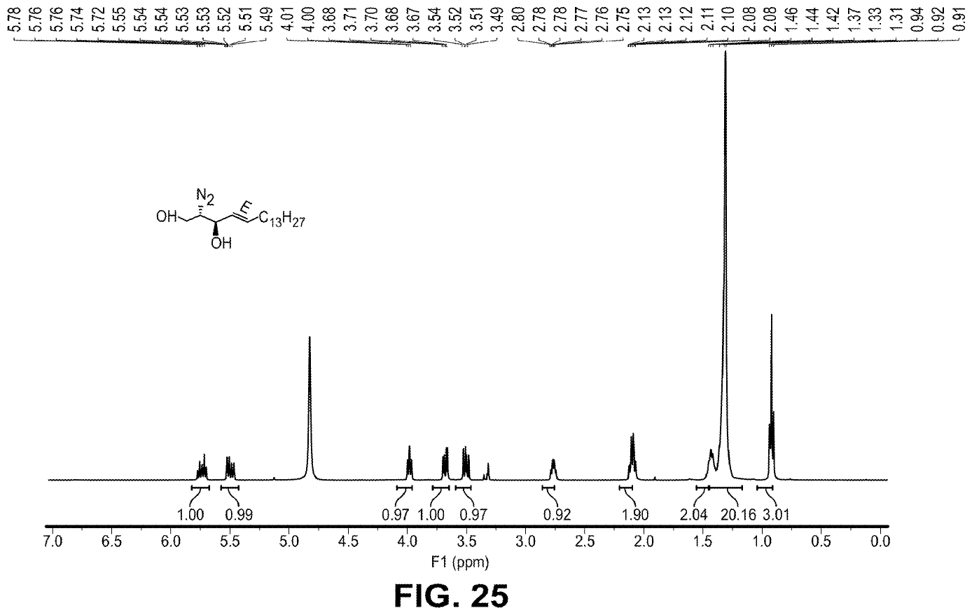

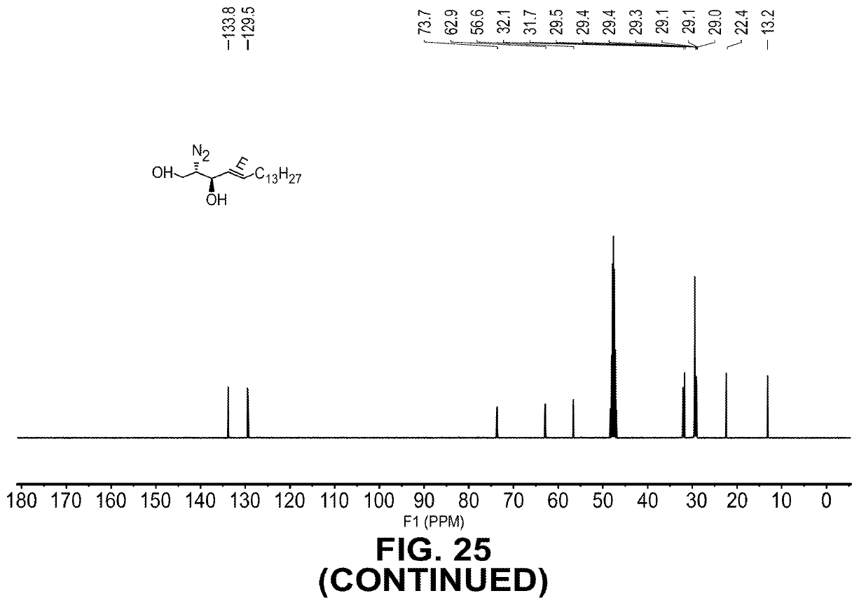

See FIG. 25 for ¹H and ¹³C NMR spectra of compound II-22b.

### Example 4: Synthesis of Additional Glycosylsphingosines

### Synthesis of O-(α-n-glucopyranosyl)-(1→1)-(2S, 3R, 4E)-2-amino-octadec-4-ene, GlcαSph (d18:1)

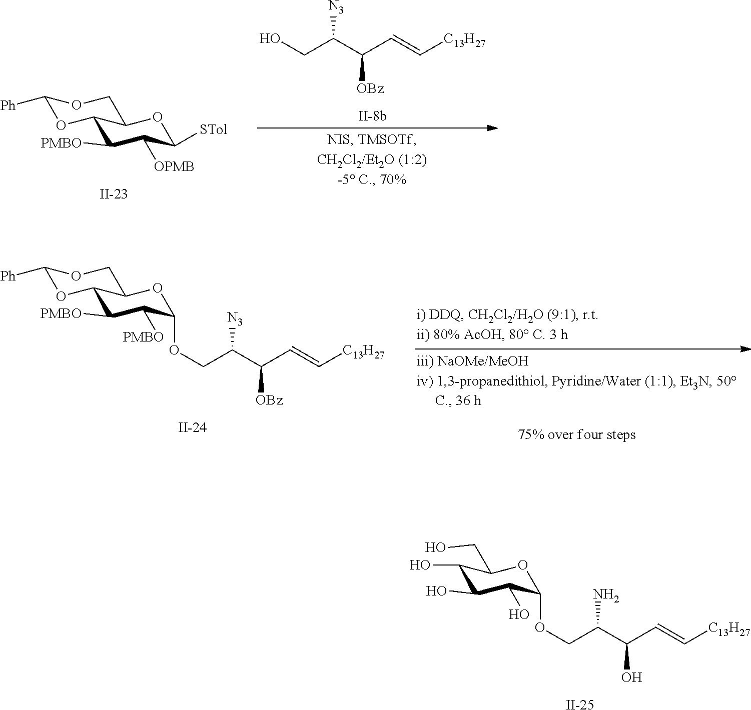

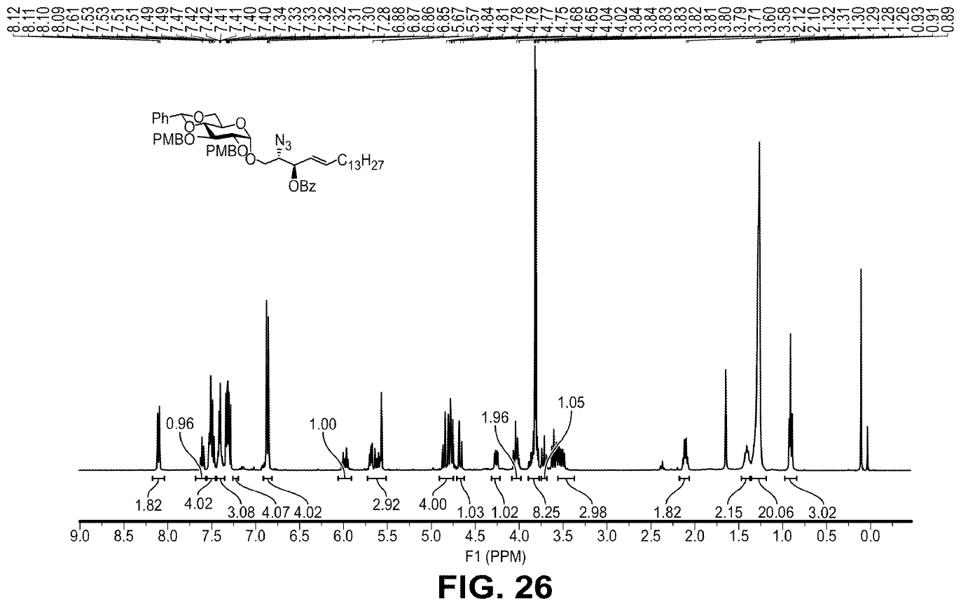

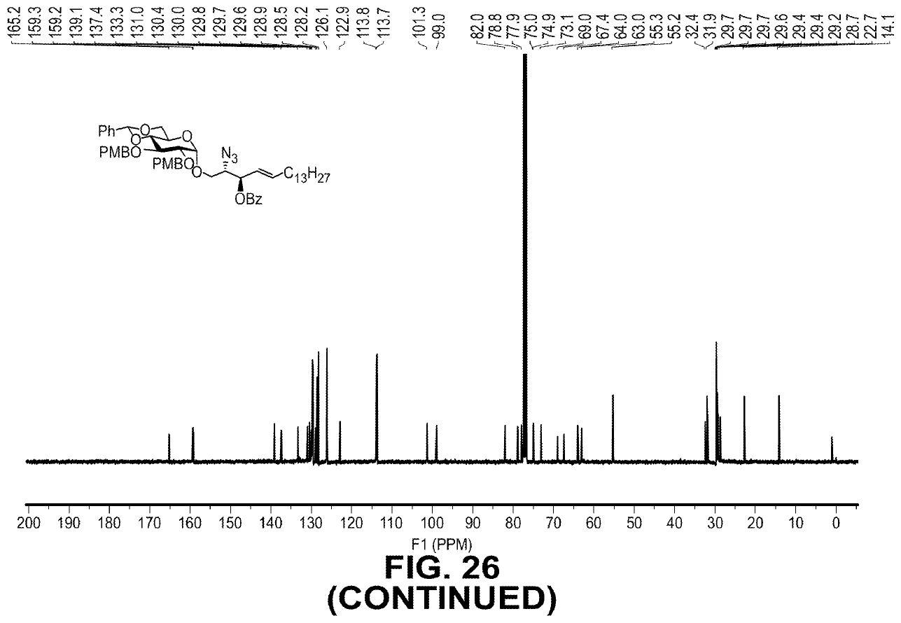

To a solution of II-8b (200 mg, 0.47 mmol, 1 eq.) and glucosyl donor II-23 (345 mg, 0.56 mmol, 1.2 eq.) in 10 mL of dry CH₂Cl₂/Et₂₀ (1:2), powdered molecular sieves (4 Å) was added. The mixture was stirred under argon at room temperature for 30 minutes. N-Iodosuccinimide (NIS) (140 mg, 0.62 mmol) was added, and the reaction mixture was cooled down to −5° C. and TMSOTf (11 μL, 0.06 mmol, 0.1 eq.) was added. The reaction mixture was stirred at −5° C. for 1 h and then the reaction was quenched with Et₃N, and the solid was filtered off. The filtrate was concentrated under vacuum, and the residue was purified by a silica gel column chromatography using hexane:EtOAc=4:1 (by volume) as an eluent to produce compound H₁-24 (300 mg) as an amorphous solid in 70% yield. ¹H NMR (400 MHz, CDCl₃) δ=6.06-5.91 (dt, J=15.1, 6.7, 1H), 5.73-5.51 (m, 3H), 4.91-4.75 (m, 4H), 4.71-4.62 (d, J=11.7, 1H), 4.31-4.22 (dd, J=10.1, 4.7, 1H), 4.08-3.98 (m, 2H), 3.89-3.77 (m, 8H), 3.75-3.68 (t, J=10.3, 1H), 3.45-3.64 (m, 3H), 2.17-2.06 (m, 2H), 1.47-1.37 (q, J=7.1, 2H), 1.36-1.19 (m, 20H), 0.97-0.84 (t, J=6.8, 3H); ¹³C NMR (100 MHz, CDCl₃) δ 165.2, 159.3, 159.2, 139.1, 137.4, 133.3, 131.0, 130.4, 130.0, 129.8, 129.7, 129.6, 128.9, 128.5, 128.2, 126.1, 122.9, 113.8, 113.7, 101.3, 99.0, 82.0, 78.8, 77.9, 75.0, 74.9, 73.1, 69.0, 67.4, 64.0, 63.0, 55.3, 55.2, 32.4, 31.9, 29.7, 29.65, 29.7, 29.6, 29.4, 29.3, 29.2, 28.7, 22.7, 14.1. See FIG. 26 for ¹H and ¹³C NMR spectra of compound I-24.

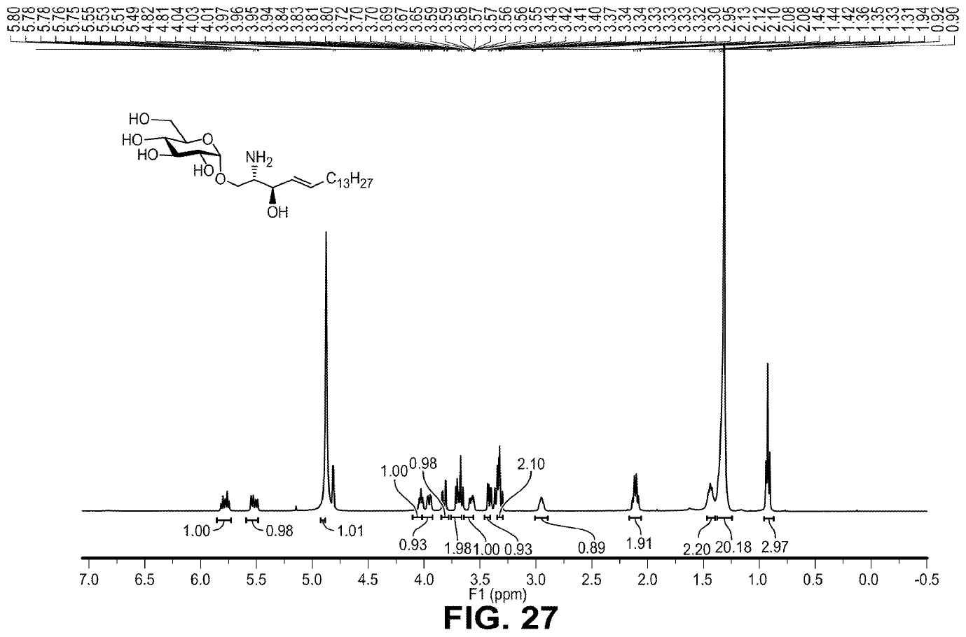

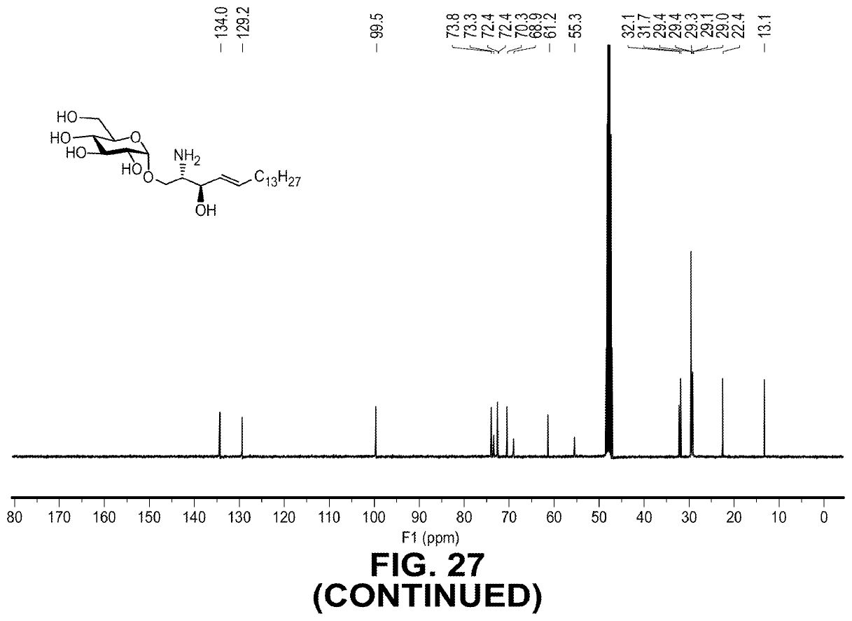

To a solution of 1-24 (300 mg, 0.33 mmol) in 6 mL of CH₂Cl₂/H₂O (9:1), DDQ (180 mg, 0.78 mmol) was added and the reaction was continued at room temperature for 4 hours. The reaction mixture was diluted with CH₂Cl₂ (30 mL) and washed with water (4×100 mL). The organic layer was separated, dried over Na₂SO₄ and evaporated to dryness. The obtained crude was dissolved in 20 mL of 80% AcOH. The mixture was heated to 80° C. and incubated at that temperature for 3 hours. The reaction mixture was co-evaporated with toluene (3×50 mL) and the obtained crude was dissolved in MeOH, 30% NaOMe in MeOH (2 mL) was then added. After being stirred at room temperature for 14 hours, the reaction mixture was neutralized with Dowex® 50W (H⁺), filtered and concentrated under a reduced pressure. This intermediate was used in the next step without further purification. To the dry intermediate in 4 mL of pyridine:water=1:1 (by volume), 1,3-propanedithiol (0.32 mL, 3.25 mmol, 10 eq.) and Et₃N (300 L) were added and the mixture was stirred at 50° C. for 36 h. The reaction mixture was concentrated and purified by a silica gel column chromatography using chloroform:methanol:water=10:8:1 (by volume) as an eluent to produce GlcαSph (d20:1) (II-25) (112 mg) as a white amorphous powder in 75% yield. ¹H NMR (400 MHz, CD₃OD) δ=5.85-5.71 (dt, J=15.4, 6.7, 1H), 5.58-5.46 (m, 1H), 4.83-4.80 (d, J=3.7, 1H), 4.07-3.99 (t, J=6.6, 1H), 3.98-3.91 (dd, J=10.0, 3.3, 1H), 3.85-3.79 (dd, J=11.9, 2.4, 1H), 3.73-3.64 (m, 2H), 3.61-3.53 (ddd, J=10.0, 5.4, 2.4, 1H), 3.43-3.38 (dd, J=9.7, 3.7, 1H), 3.36-3.31 (m, 2H), 3.00-2.91 (m, 1H), 2.15-2.07 (q, J=6.7, 2H), 1.47-1.39 (t, J=7.2, 2H), 1.40-1.25 (d, J=6.3, 20H), 0.96-0.88 (m, 3H); ¹³C NMR (100 MHz, CD₃OD) δ 134.1, 129.2, 99.5, 73.8, 73.3, 72.4, 72.5, 70.3, 68.9, 61.2, 55.3, 32.1, 31.7, 29.5, 29.4, 29.3, 29.1, 29.0, 22.4, 13.1. See FIG. 27 for ¹H and ¹³C NMR spectra of compound II-25.

### Synthesis of O-(α-n-galactopyranosyl)-(1→1)-(2S, 3R, 4E)-2-amino-octadec-4-ene, GalαSph (d18:1)

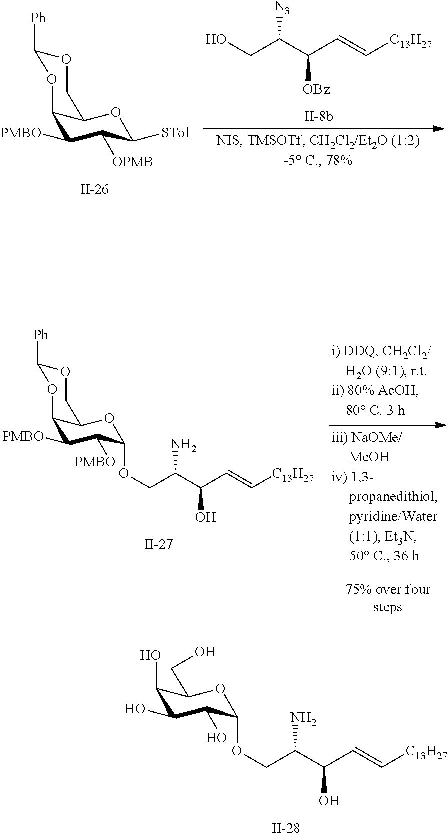

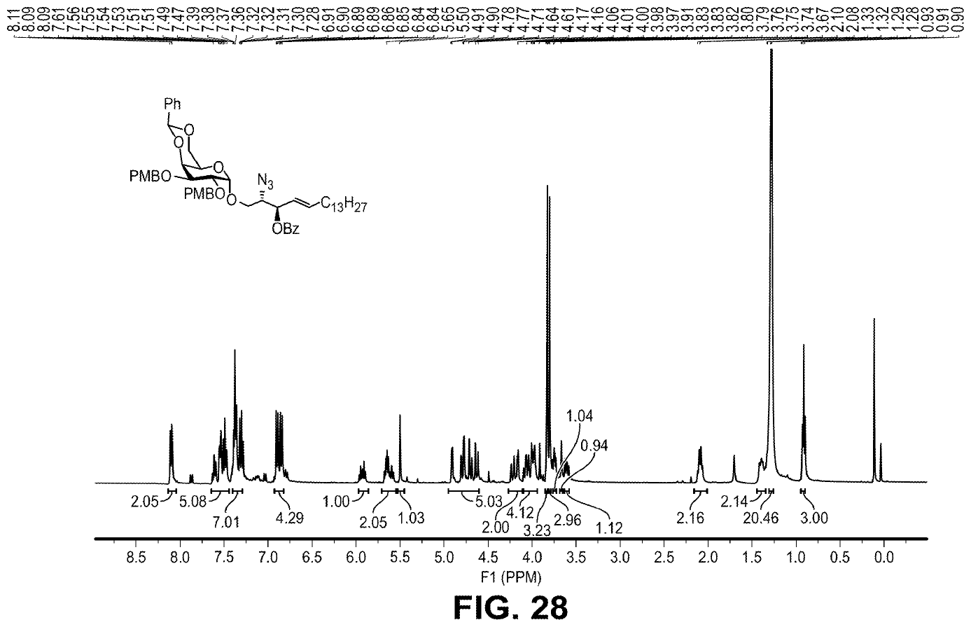

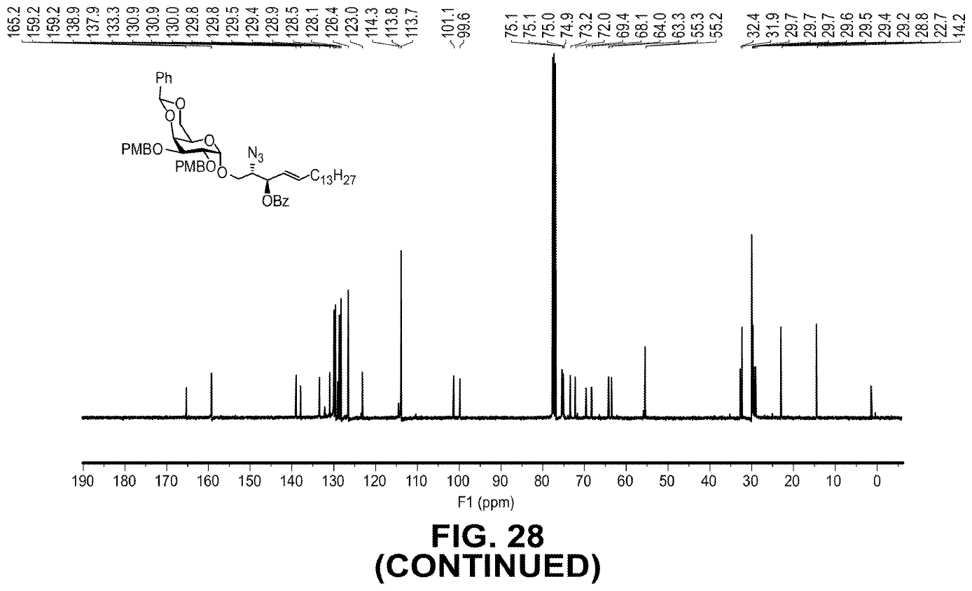

To a solution of II-8b (200 mg, 0.47 mmol, 1 eq.) and glucosyl donor II-26 (345 mg, 0.56 mmol, 1.2 eq.) in 10 mL of dry CH₂Cl₂/Et₂O (1:2), powdered molecular sieves (4 Å) was added. The mixture was stirred under argon at room temperature for 30 minutes. NIS (140 mg, 0.62 mmol) was added, and the reaction mixture was cooled down to −5° C., and TMSOTf (11 μL, 0.06 mmol, 0.1 eq.) was added. The reaction mixture was then stirred at −5° C. for 1 hour. The reaction was quenched with Et₃N, and the solid was filtered off. The filtrate was concentrated under vacuum, and the residue was purified by a silica gel column chromatography using hexane:EtOAc=4:1 (by volume) as an eluent to produce compound II-27 (334 mg) as an amorphous solid in 78% yield. ¹H NMR (400 MHz, CDCl₃) δ=8.14-8.04 (m, 2H), 7.64-7.44 (m, 5H), 7.40-7.29 (m, 7H), 6.92-6.82 (m, 4H), 5.90-5.86 (dd, J=14.4, 7.2, 1H), 5.71-5.55 (m, 2H), 5.53-5.45 (s, 1H), 4.95-4.60 (m, 5H), 4.27-4.11 (m, 2H), 4.11-3.95 (m, 4H), 3.84-3.82 (d, J=3.0, 3H), 3.81-3.79 (d, J=3.6, 3H), 3.76-3.74 (m, 1H), 3.68-3.65 (s, 1H), 3.63-3.58 (dd, J=10.8, 7.2, 1H), 2.16-2.01 (q, J=6.9, 2H), 1.45-1.35 (dd, J=11.1, 5.1, 2H), 1.31-1.26 (d, J=4.7, 20H), 0.95-0.90 (d, J=6.4, 3H); ¹³C NMR (100 MHz, CDCl₃) δ 165.2, 159.2, 159.1, 138.9, 137.9, 133.3, 130.9, 130.8, 130.0, 129.9, 129.8, 129.5, 129.4, 128.9, 128.5, 128.1, 126.4, 123.0, 114.3, 113.8, 113.7, 101.1, 99.6, 75.2, 75.1, 75.0, 74.9, 73.2, 72.0, 69.4, 68.1, 64.0, 63.3, 55.3, 55.2, 32.4, 31.9, 29.8, 29.7, 29.65, 29.6, 29.5, 29.4, 29.2, 28.8, 22.7, 14.2. See FIG. 28 for ¹H and ¹³C NMR spectra of compound H₁-27.

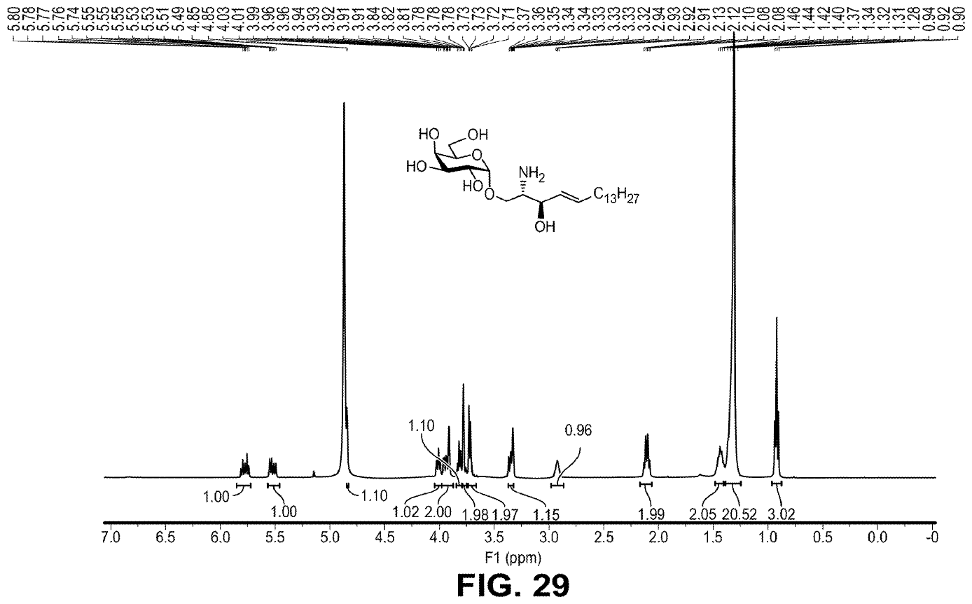

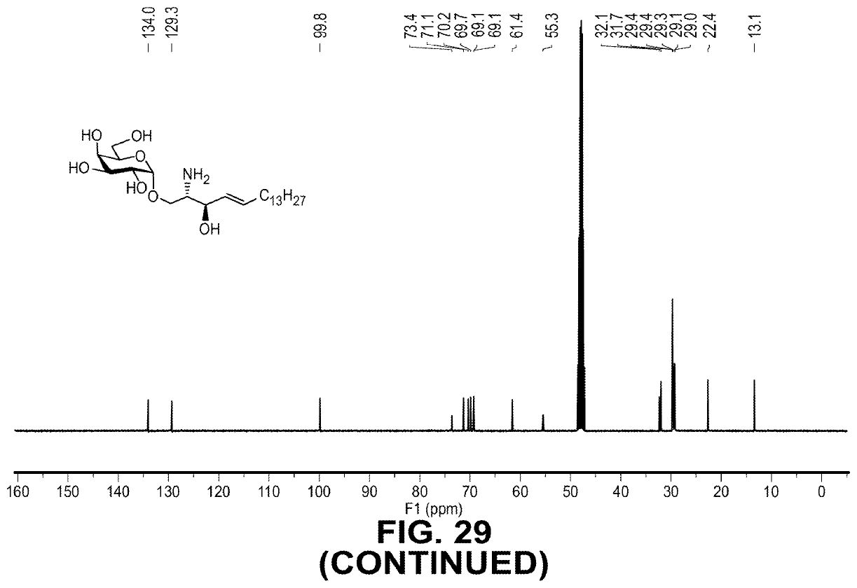

To a solution of II-27 (300 mg, 0.33 mmol) in 6 mL of CH₂Cl₂/H₂O (9:1), DDQ (180 mg, 0.78 mmol) was added and the reaction was continued at room temperature for 4 hours. The reaction mixture was diluted with CH₂Cl₂ (30 mL) and washed with water (4×100 mL). The organic layer was separated, dried over Na₂SO₄ and evaporated to dryness. The obtained crude was dissolved into 20 mL of 80% AcOH and heated at 80° C. for 3 hours. The reaction mixture was co-evaporated with toluene (3×50 mL) and the obtained crude was dissolved in MeOH, 30% NaOMe in MeOH (2 mL) was added. After being stirred at room temperature for 14 hours, the reaction mixture was neutralized with Dowex® 50W (H⁺), filtered and concentrated under a reduced pressure. This intermediate was used in the next step without further purification. To the dry intermediate in 4 mL of pyridine:water=1:1 (by volume), 1,3-propanedithiol (0.32 mL, 3.25 mmol, 10 eq.) and Et₃N (300 μL) were added and the mixture was stirred at 50° C. for 36 hours. The reaction mixture was concentrated and purified by a silica gel column chromatography using chloroform:methanol:water=10:8:1 (by volume) as an eluent to produce GalαSph (d20:1) II-28 (112 mg) as a white amorphous powder in 75% yield. ¹H NMR (400 MHz, CD₃OD) δ=5.82-5.71 (dt, J=15.4, 6.7, 1H), 5.57-5.46 (m, 1H), 4.85-4.84 (m, 1H), 4.05-3.98 (t, J=6.7, 1H), 3.98-3.88 (m, 2H), 3.85-3.79 (t, J=6.1, 1H), 3.80-3.74 (m, 2H), 3.75-3.67 (dd, J=6.0, 1.5, 2H), 3.37-3.33 (m, 1H), 2.98-2.86 (td, J=7.0, 3.3, 1H), 2.17-2.06 (q, J=6.8, 2H), 1.49-1.41 (t, J=7.2, 2H), 1.40-1.25 (s. 20H), 0.96-0.88 (t, J=6.8, 3H); ¹³C NMR (100 MHz, CD₃OD) δ 134.0, 129.3, 99.8, 73.4, 71.1, 70.2, 69.7, 69.1, 69.1, 61.4, 55.3, 32.1, 31.7, 29.4, 29.35, 13.1, 29.3, 29.1, 29.0, 22.4. See FIG. 29 for ¹H and ¹³C NMR spectra of compound II-28.

### Synthesis of O-(α-D-fucopyranosyl)-(1→1)-(2S, 3R, 4E)-2-amino-octadec-4-ene, FucαSph (d18:1)

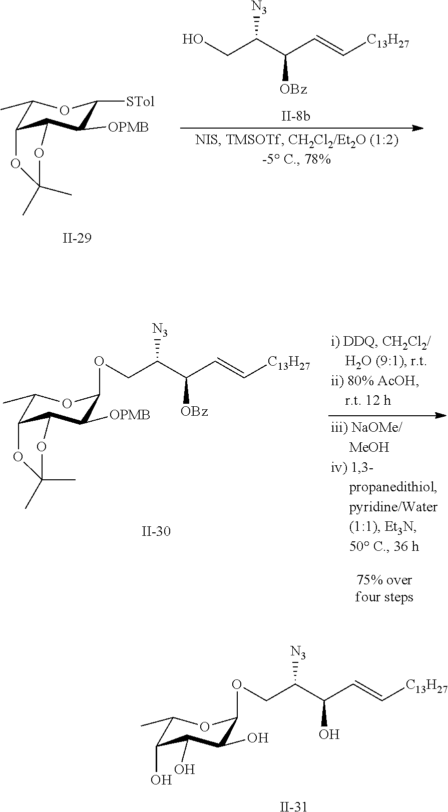

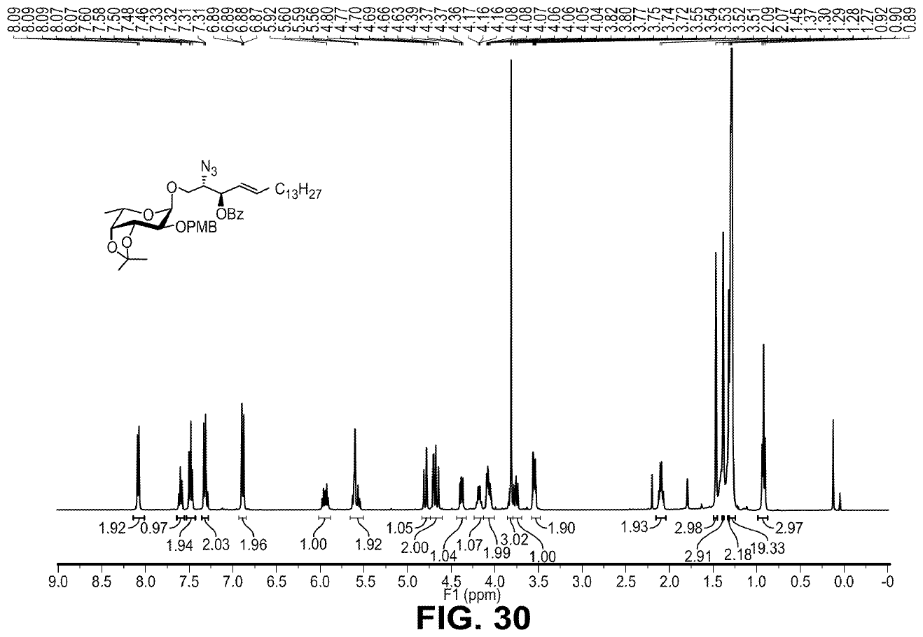

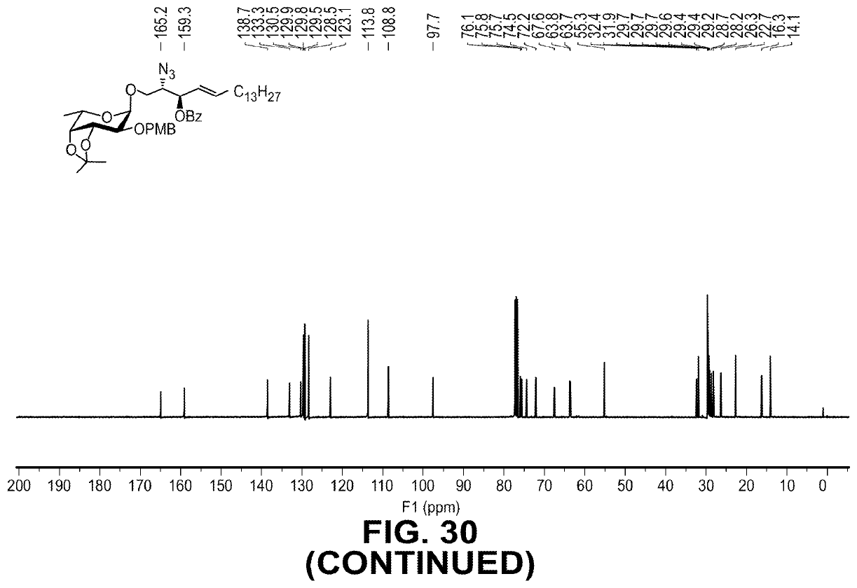

To a solution of I-8b (200 mg, 0.47 mmol, 1 eq.) and glucosyl donor II-29 (245 mg, 0.56 mmol. 1.2 eq.) in 10 mL of dry CH₂Cl₂:Et₂O=1:2 (by volume), powdered molecular sieves (4 Å) was added. The mixture was stirred under argon at room temperature for 30 minutes. To the mixture was added NIS (140 mg, 0.62 mmol). The reaction mixture was cooled down to −5° C. and TMSOTf (11 μL, 0.06 mmol. 0.1 eq.) was added. The reaction mixture was then stirred at −5° C. for 1 h. The reaction was quenched with Et₃N, and the solid was filtered off. The filtrate was concentrated under vacuum, and the residue was purified by a silica gel column chromatography using hexane:EtOAc=4:1 (by volume) as an eluent to produce compound II-30 (267 mg) as an amorphous solid in 78% yield. ¹H NMR (400 MHz, CDCl₃) δ=8.14-8.01 (m, 2H), 7.64-7.55 (d, J=7.4, 1H), 7.53-7.43 (t, J=7.7, 2H), 7.35-7.28 (m, 2H), 6.93-6.84 (m, 2H), 6.01-5.87 (dt, J=13.6, 6.8, 1H), 5.65-5.50 (m, 2H), 4.82-4.73 (d, J=12.2, 1H), 4.73-4.59 (m, 2H), 4.43-4.31 (dd, J=7.8, 5.5, 1H), 4.23-4.12 (dd, J=6.7, 2.6, 1H), 4.12-3.99 (ddd. J=12.2, 6.9, 3.4, 2H), 3.84-3.78 (s, 3H), 3.78-3.68 (dd, J=10.1, 8.1, 1H), 3.57-3.47 (m, 2H), 2.14-2.02 (q, J=6.8, 2H), 1.48-1.43 (s, 3H), 1.38-1.36 (s. 3H), 1.31-1.30 (s, 2H), 1.30-1.23 (m, 20H), 0.97-0.86 (t, J=6.8, 3H); ¹³C NMR (100 MHz, CDCl₃) δ 165.2, 159.3, 138.7, 133.3, 130.5, 129.9, 129.8, 129.5, 128.5, 123.1, 113.8, 108.8, 97.7, 76.1, 75.8, 75.7, 74.5, 72.2, 67.6, 63.8, 63.7, 55.3, 32.4, 31.9, 29.8, 29.75, 29.7, 29.6, 29.4, 29.3, 29.2, 28.7, 28.2, 26.3, 22.7, 16.3, 14.1. See FIG. 30 for ¹H and ¹³C NMR spectra of compound II-30.

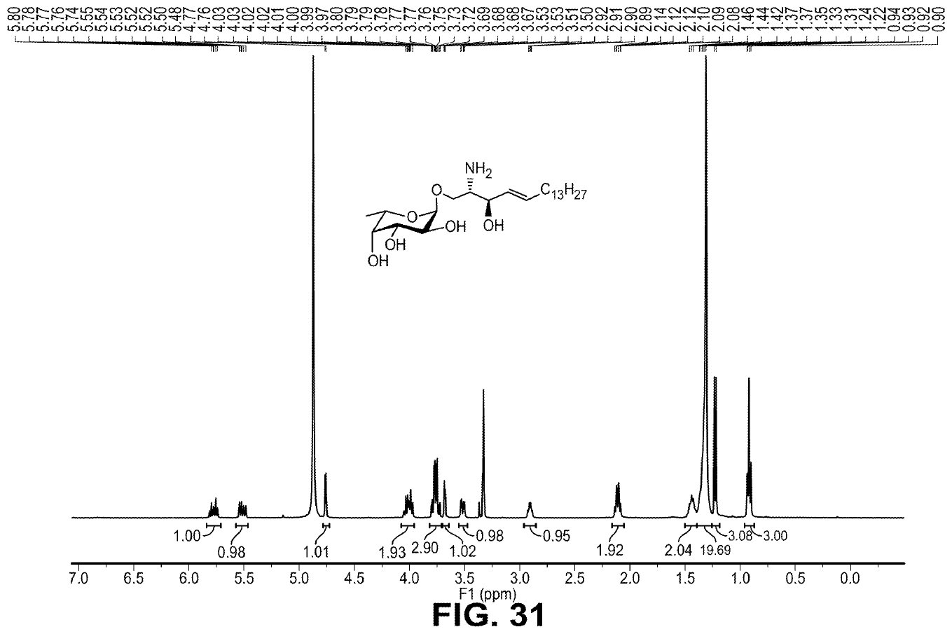

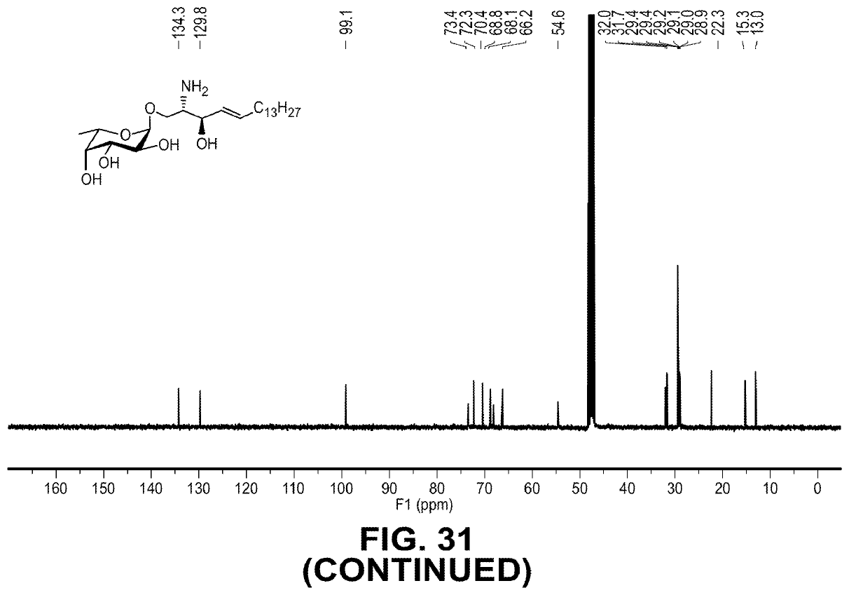

To a solution of 1-30 (250 mg, 0.34 mmol) in 6 mL of CH₂Cl₂/H₂O (9:1), DDQ (90 mg, 0.39 mmol) was added and the reaction was continued at room temperature for 4 hours. The reaction mixture was diluted with CH₂Cl₂ (30 mL) and washed with water (4×100 mL). The organic layer was separated, dried over Na₂SO₄ and evaporated to dryness. The obtained crude was dissolved in 20 mL of 80% AcOH and stirred at room temperature for 12 hours. The reaction mixture was co-evaporated with toluene (3×50 mL) and the obtained crude was dissolved in MeOH, 30% NaOMe in MeOH (2 mL) was added. After being stirred at room temperature for 14 hours, the reaction mixture was neutralized with Dowex® 50W (H⁺), filtered and concentrated under a reduced pressure. This intermediate was used in the next step without further purification. To the dry intermediate in 4 mL of pyridine:water=1:1 (by volume), 1,3-propanedithiol (0.34 mL, 3.4 mmol, 10 eq.) and Et₃N (250 L) were added and the mixture was stirred at 50° C. for 36 hours. The reaction mixture was concentrated and purified by a silica gel column chromatography using chloroform:methanol:water=10:8:1 (by volume) as an eluent to produce GalαSph (d20:1) 11-31 (113 mg) as a white amorphous powder in 75% yield. ¹H NMR (400 MHz, CD₃OD) δ=5.84-5.71 (dt, J=15.2, 6.8, 1H), 5.57-5.46 (ddt, J=15.4, 7.6, 1.4, 1H), 4.07-3.96 (m, 2H), 4.78-4.73 (d, J=3.4, 1H), 3.82-3.70 (m, 3H), 3.71-3.64 (dd, J=3.1, 1.2, 1H), 3.55-3.47 (dd, J=10.1, 3.5, 1H), 2.96-2.85 (td, J=6.5, 3.5, 1H), 2.16-2.05 (m, 2H), 1.50-1.39 (q, J=6.9, 2H), 1.38-1.26 (d, J=7.3, 20H), 1.26-1.19 (d, J=6.6, 3H), 0.96-0.87 (m, 3H); ¹³C NMR (100 MHz, CD₃OD) δ 134.3, 129.8, 99.1, 73.4, 72.3, 70.4, 68.8, 68.1, 66.2, 54.6, 32.0, 31.7, 29.5, 29.4, 29.2, 29.1, 29.0, 28.9, 22.3, 15.3, 13.0. See FIG. 31 for ¹H and ¹³C NMR spectra of compound II-31.

### Synthesis of O-(2,3,4,6-tetra-O-benzoyl-β-D-galactopyranosyl)-(1→4)-(2,3,6-tri-O-benzoyl-β-D-glucopyranosyl)-(1→1)-(2S, 3R, 4E)-2-azido-3-O-benzoyl-icos-4-ene (II-12a)

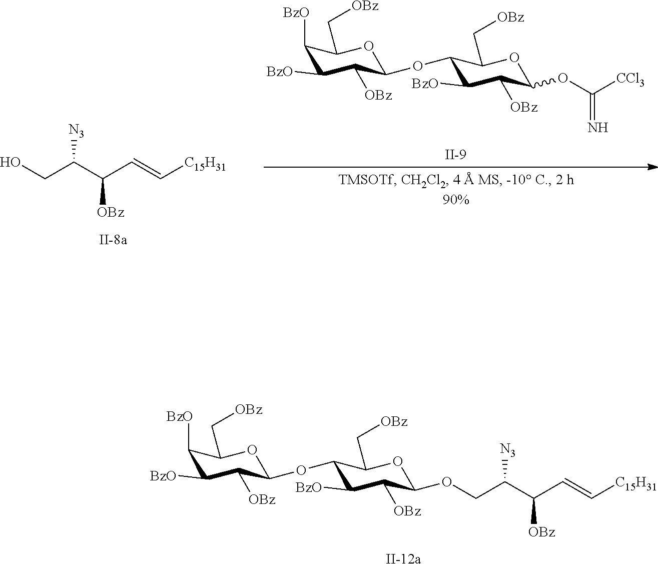

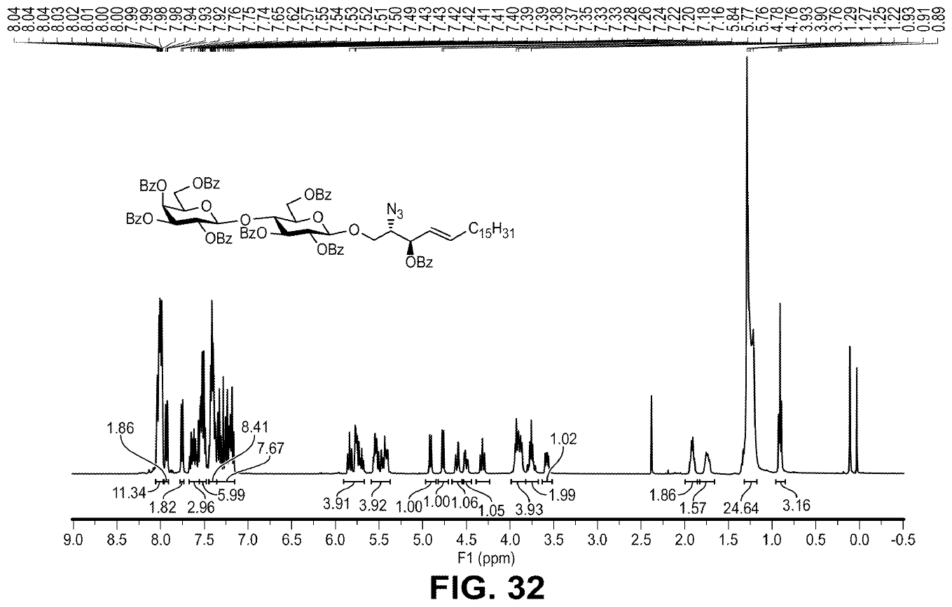

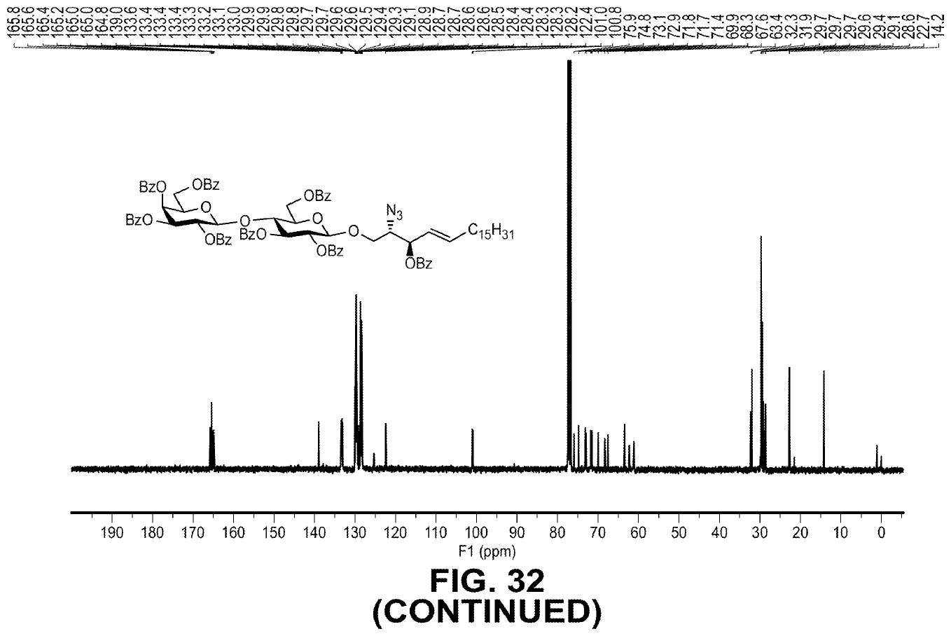

To a solution of II-8a (2 g, 4.37 mmol, 1 eq.) and per-O-benzoylated lactosyl trichloroacetimidate (II-9) (6.90 g, 5.68 mmol, 1.3 eq.) in 40 mL of dry CH₂Cl₂, powdered molecular sieves (4 Å) was added. The mixture was stirred under argon at room temperature for 30 minutes. The reaction mixture was cooled down to −10° C. and TMSOTf (104 μL, 0.57 mmol, 0.1 eq.) was added. The reaction mixture was then stirred at −10° C. for 2 h. The reaction was quenched with Et₃N, and the solid was filtered off. The filtrate was concentrated under vacuum, and the residue was purified by a silica gel column chromatography using hexane:EtOAc=4:1 (by volume) as an eluent to produce compound II-12a (5.9 g) as an amorphous solid in 90% yield. ¹H NMR (400 MHz, CDCl₃) δ=8.06-7.97 (m, 11H), 7.96-7.91 (m, 2H), 7.78-7.73 (m, 2H), 7.67-7.56 (m, 3H), 7.56-7.48 (qd, J=7.6, 6.7, 3.8, 6H), 7.45-7.36 (m, 8H), 7.36-7.15 (m, 8H), 5.90-5.67 (m, 4H), 5.59-5.37 (m, 4H), 4.97-4.85 (d, J=7.9, 1H), 4.82-4.71 (d, J=7.7, 1H), 4.67-4.55 (dd, J=12.3, 2.0, 1H), 4.54-4.44 (dd, 0.1=12.2, 4.3, 1H), 4.39-4.24 (t, J=9.5, 1H), 3.99-3.82 (ddd, J=16.4, 8.5, 4.2, 4H), 3.82-3.68 (t, J=6.4, 2H), 3.64-3.52 (dd, J=9.8, 5.6, 1H), 2.00-1.83 (q, J=6.8, 2H), 1.85-1.66 (dq, J=11.1, 3.7, 2.7, 2H), 1.32-1.18 (m, 25H), 0.96-0.85 (m, 3H); ¹³C NMR (100 MHz, CDCl₃) δ 165.8, 165.6, 165.4, 165.2, 165.5, 165.0, 164.8, 139.0, 133.6, 133.5, 133.45, 133.4, 133.3, 133.2, 133.1, 130.0, 129.95, 129.9, 129.87, 129.85, 129.8, 129.75, 129.7, 129.65, 129.6, 129.5, 129.4, 129.3, 129.1, 128.9, 128.75, 128.7, 128.65, 128.6, 128.5, 128.45, 128.4, 128.35, 128.3, 128.2, 125.3, 122.4, 101.0, 100.8, 75.9, 74.8, 73.1, 72.9, 71.8, 71.7, 71.4, 69.9, 68.3, 67.6, 63.4, 62.3, 61.1, 32.3, 31.9, 29.8, 29.75, 29.7, 29.6, 29.4, 29.1, 28.6, 22.7, 14.2. See FIG. 32 for ¹H and ¹³C NMR spectra of compound II-12a.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A method of preparing a glycosylated sphingosine, comprising:
(1a) contacting a Garner's aldehyde with an alkyne of the following structure: 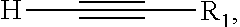
wherein R₁ is a C₁-C₂₅ alkyl,
in the presence of an organozirconium compound to form a compound of the following structure (Compound A): 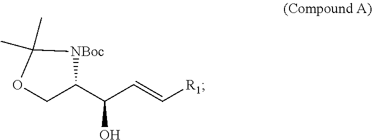
(1b) reacting the hydroxyl group of Compound A with a protecting group reagent and removing the N,O-isopropylidene acetal and tert-butyloxycarbonyl groups to form a compound of the following structure (Compound B): 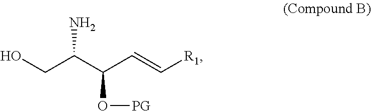
wherein PG is the protecting group;
(1c) converting the amino group of Compound B to an azido group to form a compound of the following structure (Compound C): 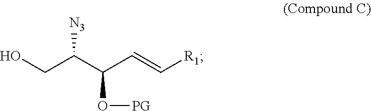
(1d) glycosylating Compound C by a trichloroacetimidate glycosylation reaction to form a compound of the following structure (Compound D): 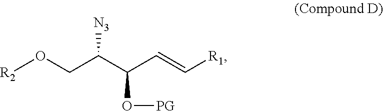
wherein R₂ is a glycosyl group; and
(1e) deprotecting one or more protecting groups present in Compound D and reducing the azido group to form a compound of the following structure (Compound E):

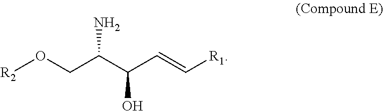

2. The method of claim 1, wherein the Garner's aldehyde is (S)-Garner's aldehyde.

3. (canceled)

4. The method of claim 1, wherein step (1a) is performed in the presence of a metallic catalyst.

5. The method of claim 4, wherein the metallic catalyst comprises a Zn-based catalyst.

6. (canceled)

7. The method of claim 1, wherein the protecting group is benzoyl.

8. (canceled)

9. The method of claim 1, wherein a purification process is performed after step (1b) and before step (1c).

10. The method of claim 1, wherein a purification process is not performed between steps (1a) and (1b).

11. The method of claim 1, wherein step (1c) is performed using triflic azide in the presence of a metal catalyst.

12. The method of claim 1, wherein the trichloroacetimidate glycosylation reaction comprises reacting Compound C and a glycosylated trichloroacetimidate in the presence of a boron catalyst.

13. (canceled)

14. The method of claim 1, wherein Compound E is selected from the group consisting of:

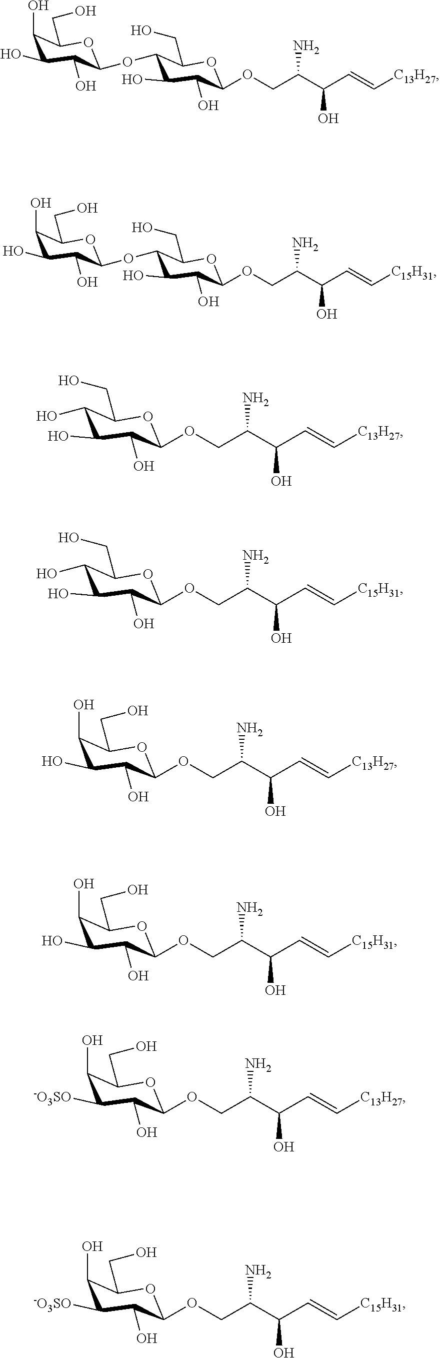

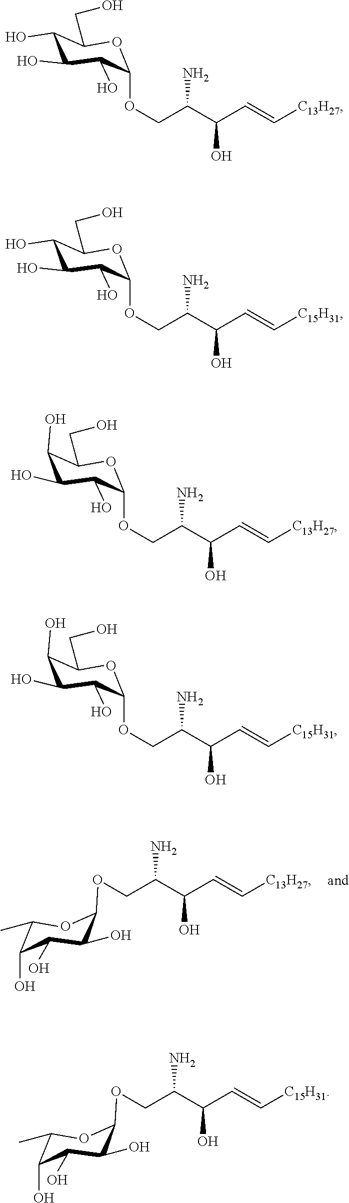

15. A glycosylated sphingosine prepared according to the method of claim 1.

16. A method of preparing a glycosylated sphingosine, comprising:
(2a) contacting a D-xylose with benzaldehyde dimethyl acetal to form a 3,5-O-benzylidene-D-xylofuranose;
(2b) cleaving the 3,5-O-benzylidene-D-xylofuranose to form a mixture of Compound F and Compound G: 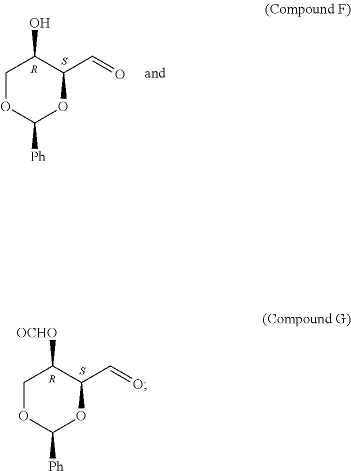
(2c) contacting the mixture of Compound F and Compound G with a triphenylphosphonium reagent of the following structure: 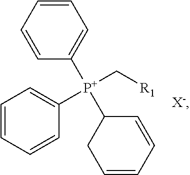
wherein R₁ is a C₁-C₂₅ alkyl and X⁻ is a counterion, to form a compound of the following structure (Compound H): 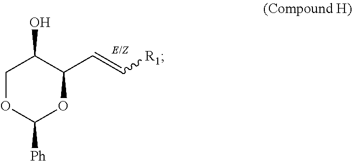
(2d) isolating the E isomer of Compound H to form a compound of the following structure (Compound I): 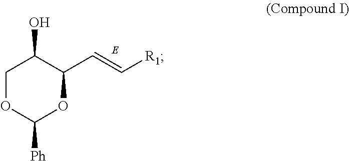
(2e) performing an azidation with S_{N}2 inversion on the hydroxyl group of Compound I to form a compound of the following structure (Compound J): 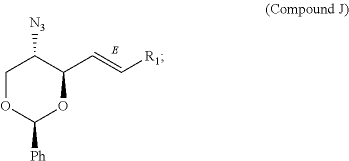
(2f) hydrolyzing Compound J to form a compound of the following structure (Compound K): 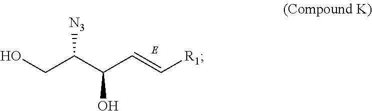
(2g) glycosylating Compound K by a trichloroacetimidate glycosylation reaction to form a compound of the following structure (Compound L): 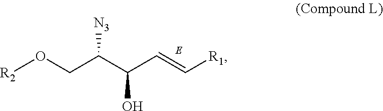
wherein R₂ is a glycosyl group; and
(2h) reducing the azido group in Compound L to form a compound of the following structure (Compound M):

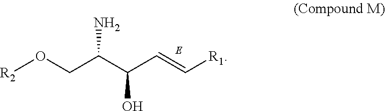

17. The method of claim 16, further comprising isomerizing the Z isomer of Compound H to form Compound I in step (2d).

18. The method of claim 16, wherein the cleaving in step (2b) is performed by a periodate oxidative cleavage reaction.

19. The method of claim 16, wherein the counterion is a halide.

20. (canceled)

21. The method of claim 16, wherein the contacting in step (2c) is performed in the presence of PhLi with or without LiBr.

22. The method of claim 17, wherein the isomerizing in step (2d) is performed by a photo-isomerization reaction.

23. The method of claim 22, wherein a light source for the photo-isomerization is sunlight or wherein a light source for the photo-isomerization is a metal halide lamp.

24. (canceled)

25. The method of claim 16, wherein the trichloroacetimidate glycosylation reaction in step (2g) comprises reacting Compound K and a glycosylated trichloroacetimidate in the presence of a promoter.

26. (canceled)

27. The method of claim 16, further comprising a step of introducing a protecting group after step (2f) and before step (2g) to result in a protected secondary alcohol.

28. The method of claim 16, wherein Compound M is selected from the group consisting of:

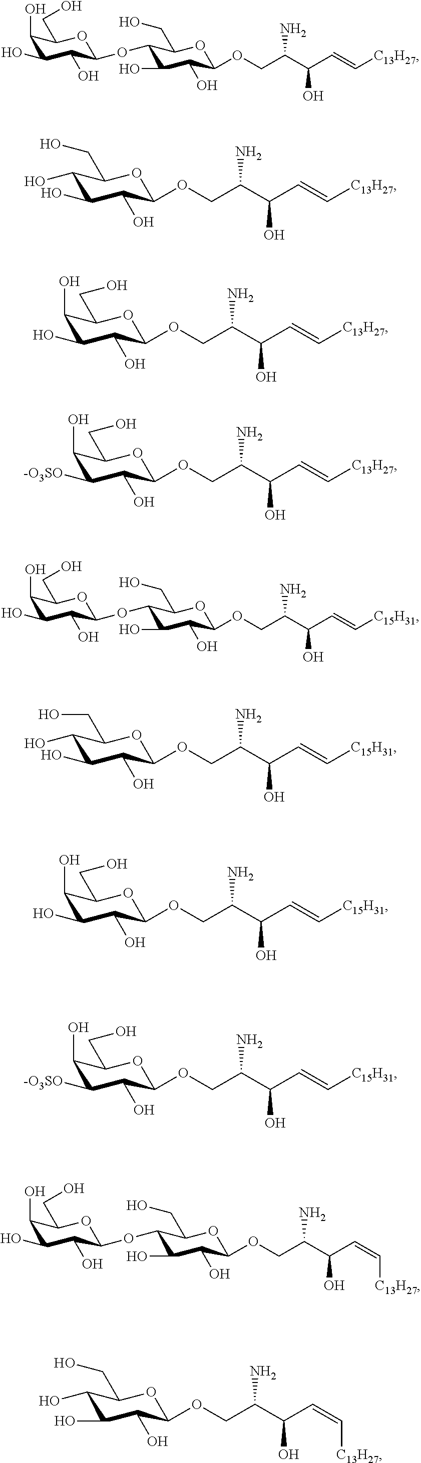

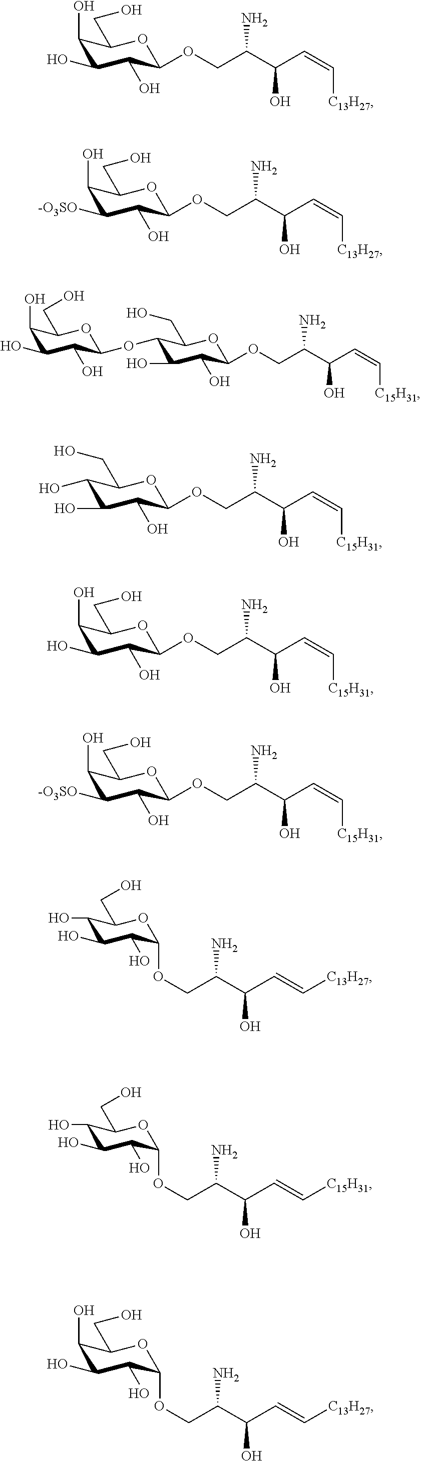

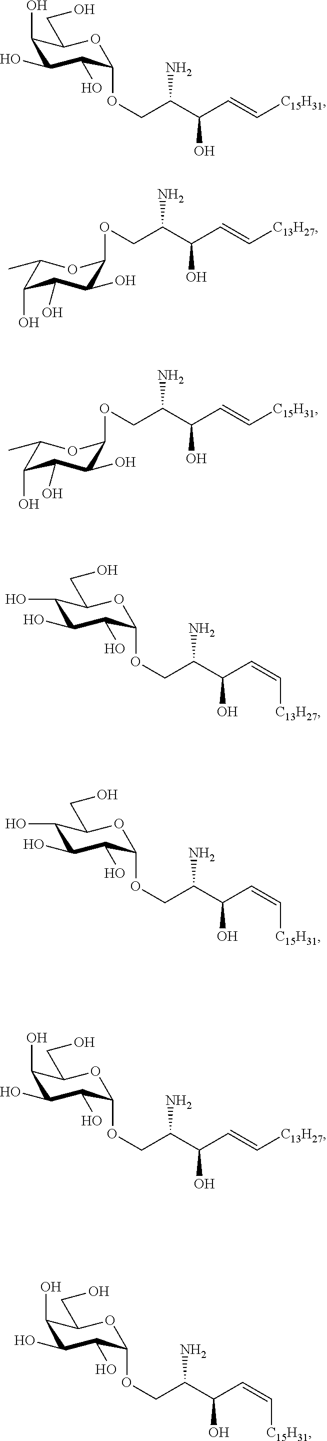

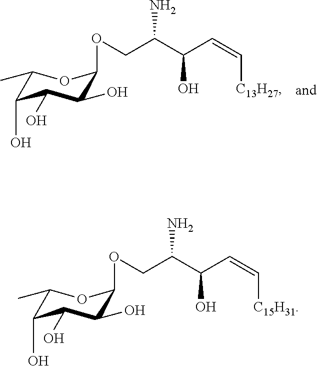

29. A glycosylated sphingosine prepared according to the method of claim 16.

30. A method of preparing a sphingosine, comprising:
(3a) contacting a Garner's aldehyde with an alkyne of the following structure: 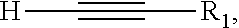
wherein R₁ is a C₁-C₂₅ alkyl,
in the presence of an organozirconium compound to form a compound of the following structure (Compound A): 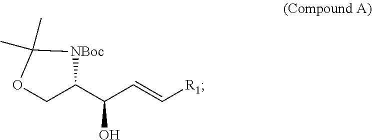
(3b) removing the N,O-isopropylidene acetal and tert-butyloxycarbonyl groups to form a compound of the following structure (Compound N):

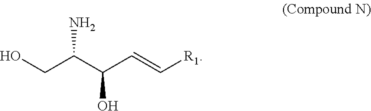

31. The method of claim 30, wherein Compound N is selected from the group consisting of:

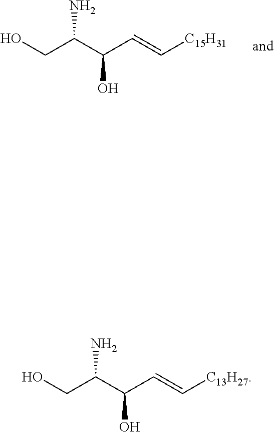

32. A method of preparing a sphingosine, comprising:
(4a) contacting a D-xylose with benzaldehyde dimethyl acetal to form a 3,5-O-benzylidene-D-xylofuranose;
(4b) cleaving the 3,5-O-benzylidene-D-xylofuranose to form a mixture of Compound F and Compound G: 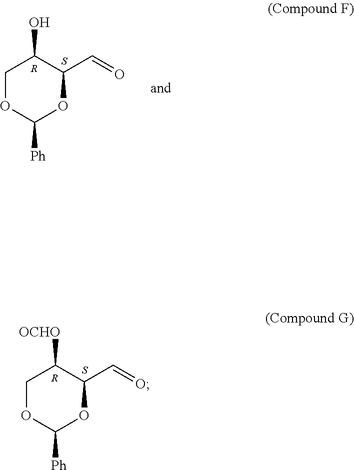
(4c) contacting the mixture of Compound F and Compound G with a triphenylphosphonium reagent of the following structure: 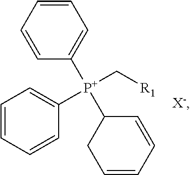
wherein R₁ is a C₁-C₂₅ alkyl and X⁻ is a counterion, to form a compound of the following structure (Compound H): 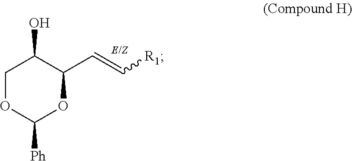
(4d) isolating the E isomer of Compound H to form a compound of the following structure (Compound I): 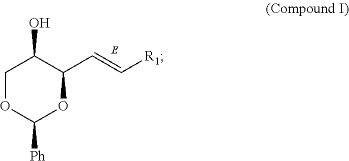
(4e) performing an azidation with S_{N}2 inversion on the hydroxyl group of Compound I to form a compound of the following structure (Compound J): 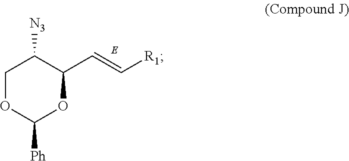
(4f) hydrolyzing Compound J to form a compound of the following structure (Compound K): 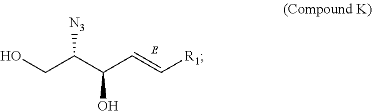
(4g) reducing the azido group in Compound L to form a compound of the following structure (Compound N):

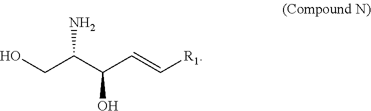

33. The method of claim 32, further comprising isomerizing the Z isomer of Compound H to form Compound I in step (4d).

34. The method of claim 32, wherein Compound N is selected from the group consisting of: [chemical structure]
